(19) United States
(12) Patent Application Publication
(10) Pub. No.: US 2024/0409963 A1
(43) Pub. Date: Dec. 12, 2024

(54) Use of Inhibitors to Increase Efficiency of Crispr/CAS Insertions

(71) Applicant: AstraZeneca AB, Sodertalje (SE)

(72) Inventors: Marcello Maresca, Sodertalje (SE); Sasa Svikovic, Sodertalje (SE); Nina Akrap, Sodertalje (SE); Sandra Wimberger, Sodertalje (SE)

(21) Appl. No.: 18/696,034

(22) PCT Filed: Apr. 6, 2023
(86) PCT No.: PCT/EP2022/077122

§ 371 Date: Mar. 27, 2024

Related U.S. Application Data
(60) Provisional application No. 63/250,945, filed on Sep. 30, 2021.

(51) Int. Cl. C12N 15/90; C12N 9/22; C12N 15/11

(52) U.S. Cl. CPC C12N 15/907; C12N 9/22; C12N 15/111; C12N 2310/20; C12N 2800/80

(57) ABSTRACT

The present disclosure provides methods of inserting a polynucleotide of interest into the genome of a eukaryotic cell, wherein said methods comprise improving the efficiency of CRISPR/Cas-mediated polynucleotide insertion by addition of an inhibitor of the microhomology-mediated end-joining (MMEJ) pathway to the eukaryotic cell. The present disclosure further provides compositions for inserting a polynucleotide of interest into the genome of a eukaryotic cell, and kits for inserting a gene of interest into the genome of a eukaryotic cell.

### FIELD OF THE INVENTION

The present disclosure provides methods of inserting a polynucleotide of interest into the genome of a eukaryotic cell, wherein said methods comprise improving the efficiency of CRISPR/Cas-mediated polynucleotide insertion by addition of an inhibitor of the microhomology-mediated end-joining (MMEJ) pathway to the eukaryotic cell. The present disclosure further provides compositions for inserting a polynucleotide of interest into the genome of a eukaryotic cell, and kits for inserting a gene of interest into the genome of a eukaryotic cell.

### BACKGROUND

The development of cost-efficient and reliable methods for precise targeted alterations to the genome of living cells has been a long-standing goal. Genome editing has the potential to eliminate genes responsible for a particular disorder (i.e. a gene “knock-out”), or alternatively, provide a means for gene manipulation or insertion to correct a genetic deficiency or enhance a biological process via a gene “knock-in.” Genome editing can be applied for treatment of a multitude of disorders, including treatment of inherited disorders, hematological disorders and cancer, and in methods of immunotherapy.

Clustered Regularly Interspaced Short Palindromic Repeats (CRISPR) and CRISPR-associated (Cas) systems are prokaryotic immune systems first discovered by Ishino in E. coli (Ishino et al., Journal of Bacteriology 169 (12): 5429-5433 (1987)). The prokaryotic immune system provides immunity against viruses and plasmids by targeting the nucleic acids of the viruses and plasmids in a sequence-specific manner. See also Soret et al., Nature Reviews Microbiology 6 (3): 181-186 (2008).

Since its original discovery, multiple groups have performed extensive research around potential applications of the CRISPR system in genetic engineering, including gene editing (Jinek et al., Science 337 (6096): 816-821 (2012); Cong et al., Science 339 (6121): 819-823 (2013); and Mali et al., Science 339 (6121): 823-826 (2013)). The CRISPR-Cas9 gene editing system has been used successfully in a wide range of organisms and cell lines. In addition to genome editing, the CRISPR system has a multitude of other applications, including regulating gene expression, genetic circuit construction, and functional genomics, amongst others (reviewed in Sander et al., Nature Biotechnology 32:347-355 (2014)).

The Cas9 endonuclease generates a double-stranded DNA break at the target sequence, upstream of a protospacer adjacent motif (PAM). The target sequence can then be removed, or a sequence of interest can be inserted into the target sequence using an endogenous repair pathway of the cell. Endogenous DNA repair pathways include the Non-Homologous End Joining (NHEJ) pathway, Microhomology-Mediated End Joining (MMEJ) pathway, and the Homology Directed Repair (HDR) pathway. NHEJ, MMEJ, and HDR pathways repair double-stranded DNA breaks, but repair of such double-stranded DNA breaks may result in insertions or deletions at the double-stranded break site. In NHEJ, a homologous template is not required for repairing breaks in the DNA. NHEJ repair can be error-prone, although errors are decreased when the DNA break includes compatible overhangs. NHEJ and MMEJ are mechanistically distinct DNA repair pathways with different subsets of DNA repair enzymes involved in each of them. Unlike NHEJ, which can be precise in some cases, or error-prone in some cases, MMEJ is always error-prone and results in both deletion and insertions at the site under repair. MMEJ-associated deletions are due to the micro-homologies (2-10 base pairs) at both sides of a double-strand break. In contrast, HDR requires a homologous template to direct repair, but HDR repairs are typically high-fidelity and less error-prone. HDR-driven repair of double-stranded DNA breaks is therefore preferable to NHEJ- or MMEJ-mediated repair; however, in many cell types HDR is limited by the activity of NHEJ at all cell cycle stages, and HDR is primarily utilized in the S phase of cell growth (Mao et al., Cell Cycle, 7:2902-2906 (2008)).

### SUMMARY OF THE INVENTION

In some embodiments, the present disclosure relates to methods of increasing the efficiency of CRISPR/Cas-mediated gene insertion. In some embodiments, the method comprises inserting a polynucleotide of interest into the genome of a eukaryotic cell, the method comprising (a) adding an inhibitor of the MMEJ pathway to a composition comprising the eukaryotic cell, (b) adding a Cas effector protein to the composition, and (c) adding the polynucleotide of interest to the composition, wherein the polynucleotide of interest is inserted into the genome of the eukaryotic cell by homology directed repair (HDR) or single-stranded template repair (SSTR).

In some embodiments, step (a) of the method further comprises adding an inhibitor of the non-homologous end-joining (NHEJ) pathway.

In some embodiments, the method further comprises (d) adding a polynucleotide comprising an RNA guide sequence, a Cas-binding region, a DNA template sequence, or combinations thereof to the composition.

In some embodiments, the Cas effector protein and the polynucleotide of (d) are added in the form of a ribonucleoprotein (RNP).

In some embodiments, the Cas effector protein is added in (b) by adding a Cas polynucleotide encoding the Cas effector protein.

In some embodiments, the polynucleotide of interest, the polynucleotide of step (d) and the Cas polynucleotide are encoded on a single vector. In some embodiments, the polynucleotide of interest is added as DNA. In some embodiments, the polynucleotide of step (d) is added as DNA. In some embodiments, the polynucleotide of step (d) is added as RNA. In some embodiments, the Cas effector polynucleotide is added as DNA. In some embodiments, the Cas polynucleotide is added as RNA. In some embodiments, the Cas polynucleotide is added as mRNA.

In some embodiments, the vector is a viral vector. In some embodiments, the viral vector is a retrovirus, a lentivirus, an adenovirus, or an adeno-associated virus (AAV).

In some embodiments, the Cas effector protein, the polynucleotide of interest, and the polynucleotide of (d) are added to the eukaryotic cell by microinjection, electroporation, or via a lipid nanoparticle, liposome, exosome, gold nanoparticle or a DNA nanoclew.

In some embodiments, the vector is added to the composition comprising the eukaryotic cell by transfecting the eukaryotic cell.

In some embodiments, the Cas effector protein is a Cas9 nuclease, a Cas12a nuclease, or a Cas12f nuclease. In some embodiments, the Cas effector protein is a Cas9 nuclease. In some embodiments, the Cas9 nuclease is a Cas9 nuclease fused to a reverse transcriptase, a Cas9 nuclease fused to a DNA polymerase, a Cas9 nuclease fused to DN1S, a Cas9 nickase, a Cas9 fused to a Geminin degron domain, or a Cas9 nuclease fused to CTIP.

In some embodiments, the polynucleotide of interest is added via a vector. In some embodiments, the vector is a viral vector. In some embodiments, the viral vector is a retrovirus, a lentivirus, an adenovirus, or an adeno-associated virus (AAV).

In some embodiments, the polynucleotide of interest comprises a gene of interest. In some embodiments, the polynucleotide of interest is 1 to 50 base pairs in length. In some embodiments, the polynucleotide of interest is 1 to 10 base pairs in length. In some embodiments, the polynucleotide of interest is 50 to 5000 base pairs in length.

In some embodiments, the polynucleotide of interest is single-stranded. In some embodiments, the polynucleotide of interest is double stranded. In some embodiments, the polynucleotide of interest is a hybrid polynucleotide comprising single-stranded and double-stranded regions. In some embodiments, the hybrid polynucleotide comprises double-stranded sequences at the 5′ and 3′ ends and an internal single-stranded sequence. In some embodiments, the polynucleotide of interest is double-stranded with blunt ends. In some embodiments, the polynucleotide of interest is double-stranded with a 3′ overhang. In some embodiments, the polynucleotide of interest is double-stranded with a 5′ overhang. In some embodiments, the polynucleotide of interest is a circular polynucleotide.

In some embodiments, the polynucleotide of interest comprises a chemical modification which enhances the activity, distribution, or uptake of the polynucleotide.

In some embodiments, the inhibitor of the MMEJ pathway is an inhibitor of POL Q/DNA polymerase θ. In some embodiments, the inhibitor of POL Q is PolQ 1, PolQ 2, PolQ 3, PolQ 4, PolQ 5, PolQ 6 PolQ 7, or combinations thereof. In some embodiments, the inhibitor of POL Q is a peptide.

In some embodiments, the inhibitor of the MMEJ pathway in the composition comprising the eukaryotic cell is about 0.01 μM to about 1 mM, about 0.1 μM to about 1 mM, about 0.1 μM to about 0.5 mM, about 0.1 μM to about 100 μM, or about 1 μM to about 50 μM.

In some embodiments, the inhibitor of the NHEJ pathway is an inhibitor of DNA-dependent protein kinase (DNA-PK). In some embodiments, the inhibitor of DNA-PK is M3814, M9831/VX984, Nu7441, KU0060648, AZD7648, or combinations thereof. In some embodiments, the inhibitor of DNA-PK is AZD7648. In some embodiments, the inhibitor of DNA-PK is a peptide.

In some embodiments, the inhibitor of the NHEJ pathway in the composition comprising the eukaryotic cell is about 0.01 μM to about 1 mM, about 0.1 μM to about 1 mM, about 0.1 μM to about 0.5 mM, about 0.1 μM to about 100 μM, or about 1 μM to about 50 μM.

In some embodiments, the inhibitor of the MMEJ pathway is added to the composition comprising the eukaryotic cell 0 minutes to about 48 hours, 0 minutes to about 24 hours, 0 minutes to about 12 hours, 0 minutes to about 6 hours, or 0 minutes to about 1 hour before the Cas effector protein is added to the composition. In some embodiments, the inhibitor of the MMEJ pathway is added to the composition comprising the eukaryotic cell 0 minutes to about 1 hour after the Cas effector protein is added to the composition comprising the eukaryotic cell.

In some embodiments, the inhibitor of the NHEJ pathway is added to the composition comprising the eukaryotic cell 0 minutes to about 48 hours, 0 minutes to about 24 hours, 0 minutes to about 12 hours, 0 minutes to about 6 hours, or 0 minutes to about 1 hour before the Cas effector protein is added to the composition. In some embodiments, the inhibitor of the NHEJ pathway is added to the composition comprising the eukaryotic cell 0 minutes to about 1 hour after the Cas effector protein is added to the composition comprising the eukaryotic cell.

In some embodiments, the inhibitor of the MMEJ pathway and the inhibitor of the NHEJ pathway are added to the composition comprising the eukaryotic cell at the same time. In some embodiments, the inhibitor of the MMEJ pathway and the inhibitor of the NHEJ pathway are added to the composition comprising the eukaryotic cell at different times.

In some embodiments, the inhibitor of the MMEJ pathway, the inhibitor of the NHEJ pathway, and the Cas effector protein are added to the composition comprising the eukaryotic cell at the same time.

In some embodiments, the inhibitor of the MMEJ pathway is in the composition comprising the eukaryotic cell for about 1 to about 300 hours, for about 10 to about 100 hours, or about 20 to about 80 hours.

In some embodiments, the inhibitor of the MMEJ pathway is added to the composition comprising the eukaryotic cell at least once, at least twice, or at least three times.

In some embodiments, the inhibitor of the NHEJ pathway is in the composition comprising the eukaryotic cell for about 1 to about 300 hours, for about 10 to about 100 hours, or about 20 to about 80 hours.

In some embodiments, the inhibitor of the NHEJ pathway is added to the composition comprising the eukaryotic cell at least once, at least twice, or at least three times.

In some embodiments, the composition comprising the eukaryotic cell is a cell culture. In some embodiments, the cell culture is an in vitro cell culture or an ex vivo cell culture. In some embodiments, the eukaryotic cell is in vivo.

In some embodiments, the cell culture comprises a cell extract.

In some embodiments, the eukaryotic cell is a lymphocyte. In some embodiments, the lymphocyte comprises a chimeric antigen receptor (CAR) or a T cell receptor (TCR).

In some embodiments, the eukaryotic cell is a pluripotent stem cell. In some embodiments, the pluripotent stem cell is an induced pluripotent stem cell (iPSC).

In some embodiments, the cell culture is a mammalian cell culture.

In some embodiments, the present disclosure relates to methods of increasing the efficiency of CRISPR/Cas-mediated gene insertion comprising inserting a polynucleotide of interest into a genome of a eukaryotic cell comprising a genomically-integrated Cas polynucleotide. In some embodiments, the disclosure provides a method of inserting a polynucleotide of interest into a genome of a eukaryotic cell, the method comprising: (a) adding an inhibitor of the microhomology-mediated end joining (MMEJ) pathway to a composition comprising the eukaryotic cell, and (b) adding the polynucleotide of interest to the composition, wherein the genome comprises a genomically integrated Cas polynucleotide, and wherein the polynucleotide of interest is inserted into the genome by homology directed repair (HDR) or single-stranded template repair (SSTR). In some embodiments, the genomically-integrated Cas polynucleotide is inducible.

In some embodiments, the method further comprises adding an inhibitor of the non-homologous end joining (NHEJ) pathway to the composition.

In some embodiments, the method further comprises (c) adding a polynucleotide comprising an RNA guide sequence, a Cas-binding region, a DNA template sequence, or combinations thereof, to the composition.

In some embodiments, (i) the polynucleotide of interest and (ii) the polynucleotide of (c) are encoded on a vector. In some embodiments, the polynucleotide of interest is added as DNA. In some embodiments, the polynucleotide of (c) is added as DNA. In some embodiments, the polynucleotide of (c) is added as RNA.

In some embodiments, the vector is a viral vector. In some embodiments, the viral vector is a retrovirus, a lentivirus, an adenovirus, or an adeno-associated virus (AAV). In some embodiments, the vector is added to the composition comprising the eukaryotic cell by transfecting the eukaryotic cell.

In some embodiments, the Cas effector protein is a Cas9 nuclease, a Cas12a nuclease, or a Cas12f nuclease. In some embodiments, the Cas effector protein is a Cas9 nuclease. In some embodiments, the Cas9 nuclease is a Cas9 nuclease fused to a reverse transcriptase, a Cas9 nuclease fused to a DNA polymerase, a Cas9 nuclease fused to DN1S, a Cas9 nickase, a Cas9 fused to a Geminin degron domain, or a Cas9 nuclease fused to CTIP.

In some embodiments, the polynucleotide of interest is added via a vector. In some embodiments, the vector is a viral vector. In some embodiments, the viral vector is a retrovirus, a lentivirus, an adenovirus, or an adeno-associated virus (AAV).

In some embodiments, the polynucleotide of interest comprises a gene of interest. In some embodiments, the polynucleotide of interest is 1 to 50 base pairs in length, 1 to 10 base pairs in length, or 50 to 5000 base pairs in length.

In some embodiments, the polynucleotide of interest is single-stranded. In some embodiments, the polynucleotide of interest is double stranded. In some embodiments, the polynucleotide of interest is a hybrid polynucleotide comprising single-stranded and double-stranded regions. In some embodiments, the hybrid polynucleotide comprises double-stranded sequences at the 5′ and 3′ ends and an internal single-stranded sequence. In some embodiments, the polynucleotide of interest is double-stranded with blunt ends. In some embodiments, the polynucleotide of interest is double-stranded with a 3′ overhang. In some embodiments, the polynucleotide of interest is double-stranded with a 5′ overhang. In some embodiments, the polynucleotide of interest is a circular polynucleotide.

In some embodiments, the polynucleotide comprises a chemical modification which enhances the activity, distribution, or uptake of the polynucleotide.

In some embodiments, the inhibitor of the MMEJ pathway is an inhibitor of POL Q/DNA polymerase θ. In some embodiments, the inhibitor of POL Q is PolQ 1, PolQ 2, PolQ 3, PolQ 4, PolQ 5, PolQ 6 PolQ 7, or combinations thereof. In some embodiments, the inhibitor of POL Q is a peptide.

In some embodiments, the inhibitor of the MMEJ pathway in the composition comprising the eukaryotic cell is about 0.01 μM to about 1 mM, about 0.1 μM to about 1 mM, about 0.1 μM to about 0.5 mM, about 0.1 μM to about 100 μM, or about 1 μM to about 50 μM.

In some embodiments, the inhibitor of the NHEJ pathway is an inhibitor of DNA-dependent protein kinase (DNA-PK). In some embodiments, the inhibitor of DNA-PK is M3814, M9831/VX984, Nu7441, KU0060648, AZD7648, or combinations thereof. In some embodiments, the inhibitor of DNA-PK is AZD7648. In some embodiments, the inhibitor of DNA-PK is a peptide.

In some embodiments, the inhibitor of the NHEJ pathway in the composition comprising the eukaryotic cell is about 0.01 μM to about 1 mM, about 0.1 μM to about 1 mM, about 0.1 μM to about 0.5 mM, about 0.1 μM to about 100 μM, or about 1 μM to about 50 μM.

In some embodiments, the inhibitor of the MMEJ pathway is added to the composition comprising a eukaryotic cell comprising a genomically-integrated Cas polynucleotide 0 minutes to about 48 hours, 0 minutes to about 24 hours, 0 minutes to about 12 hours, 0 minutes to about 6 hours, or 0 minutes to about 1 hour before induction of the genomically-integrated Cas polynucleotide.

In some embodiments, the inhibitor of the NHEJ pathway is added to the composition comprising a eukaryotic cell comprising a genomically-integrated Cas polynucleotide 0 minutes to about 48 hours, 0 minutes to about 24 hours, 0 minutes to about 12 hours, 0 minutes to about 6 hours, or 0 minutes to about 1 hour before induction of the genomically-integrated Cas polynucleotide.

In some embodiments, the inhibitor of the MMEJ pathway and the inhibitor of the NHEJ pathway are added to the composition comprising the eukaryotic cell comprising a genomically-integrated Cas polynucleotide at the same time. In some embodiments, the inhibitor of the MMEJ pathway and the inhibitor of the NHEJ pathway are added to the composition comprising the eukaryotic cell comprising a genomically-integrated Cas polynucleotide at different times.

In some embodiments, the inhibitor of the MMEJ pathway is added to the composition comprising a eukaryotic cell comprising a genomically-integrated Cas polynucleotide at the same time as induction of the genomically-integrated Cas polynucleotide.

In some embodiments, the inhibitor of the NHEJ pathway is added to the composition comprising a eukaryotic cell comprising a genomically-integrated Cas polynucleotide at the same time as induction of the genomically-integrated Cas polynucleotide

In some embodiments, the inhibitor of the MMEJ pathway and the inhibitor of the NHEJ pathway are added to the composition comprising a eukaryotic cell comprising a genomically-integrated Cas polynucleotide at the same time as induction of the genomically-integrated Cas polynucleotide.

In some embodiments, the inhibitor of the MMEJ pathway is in the composition comprising the eukaryotic cell comprising a genomically-integrated Cas polynucleotide for about 1 to about 300 hours, about 10 to about 100 hours, or about 20 to about 80 hours.

In some embodiments, the inhibitor of the MMEJ pathway is added to the composition comprising the eukaryotic cell comprising a genomically-integrated Cas polynucleotide at least once, at least twice, or at least three times.

In some embodiments, the inhibitor of the NHEJ pathway is in the composition comprising the eukaryotic cell comprising a genomically-integrated Cas polynucleotide for about 1 to about 300 hours, about 10 to about 100 hours, or about 20 to about 80 hours.

In some embodiments, the inhibitor of the NHEJ pathway is added to the composition comprising the eukaryotic cell comprising a genomically-integrated Cas polynucleotide at least once, at least twice, or at least three times.

In some embodiments, the composition comprising the eukaryotic cell comprising a genomically-integrated Cas polynucleotide is a cell culture. In some embodiments, the cell cultures is an in vitro cell culture or an ex vivo cell culture.

In some embodiments, the eukaryotic cell comprising a genomically-integrated Cas polynucleotide is in vivo.

In some embodiments, the cell culture comprises a cell extract. In some embodiments, the cell culture is a mammalian cell culture.

In some embodiments, the eukaryotic cell comprising a genomically-integrated Cas polynucleotide is a lymphocyte. In some embodiments, the lymphocyte comprises a chimeric antigen receptor (CAR) or a T cell receptor (TCR).

In some embodiments, the eukaryotic cell comprising a genomically-integrated Cas polynucleotide is a pluripotent stem cell. In some embodiments, the pluripotent stem cell is an induced pluripotent stem cell (iPSC).

In some embodiments, the present disclosure relates to a method of inserting a polynucleotide of interest into a genome of a eukaryotic cell, the method comprising (a) adding an inhibitor of the microhomology-mediated end joining (MMEJ) pathway to a composition comprising the eukaryotic cell, and (b) adding to the composition comprising the eukaryotic cell (i) a Cas effector protein, (ii) a polynucleotide of interest, and (iii) a polynucleotide comprising an RNA guide sequence, a Cas-binding region, a DNA template sequence, or combinations thereof, wherein the polynucleotide of interest is inserted into the genome by homology directed repair (HDR) or single-stranded template repair (SSTR).

In some embodiments, the method comprises adding an inhibitor of the non-homologous end joining (NHEJ) pathway to the composition comprising the eukaryotic cell.

In some embodiments, the Cas effector protein and the polynucleotide comprising an RNA guide sequence, a Cas-biding region, a DNA template sequence, or combinations thereof, are added in the form of a ribonucleoprotein (RNP).

In some embodiments, the Cas effector protein is encoded by a Cas polynucleotide. In some embodiments, the Cas effector protein and the polynucleotide of interest are encoded on a vector. In some embodiments, the Cas effector protein and the polynucleotide of (iii) are encoded on a vector. In some embodiments, the Cas effector protein, the polynucleotide of interest, and the polynucleotide of (iii) are encoded on a vector. In some embodiments, the polynucleotide is on a vector.

In some embodiments, the present disclosure relates to a method of increasing the efficiency of homology directed repair (HDR) and single-stranded template repair (SSTR) gene insertions in a eukaryotic cell, the method comprising adding an inhibitor of the microhomology-mediated end joining (MMEJ) pathway when performing CRISPR/Cas-mediated gene insertions in the eukaryotic cell.

In some embodiments, the method further comprises adding an inhibitor of the non-homologous end joining (NHEJ) pathway.

In some embodiments, the CRISPR/Cas-mediated gene insertion is a CRISPR/Cas9-mediated gene insertion.

In some embodiments, the present disclosure relates to a method of reducing microhomology-mediated end joining (MMEJ) pathway recombination during CRISPR/Cas-mediated gene insertion in a cell, the method comprising adding an inhibitor of the MMEJ pathway to the cell when performing Cas-mediated gene insertions.

In some embodiments, the method further comprises reducing non-homologous end joining (NHEJ) recombination during CRISPR/Cas-mediated gene insertions in a cell comprising adding an inhibitor of the NHEJ pathway to the cell.

In some embodiments, the CRISPR/Cas-mediated gene insertions are CRISPR/Cas9-mediated gene insertions.

In some embodiments, the present disclosure relates to a composition comprising a Cas effector protein or a vector encoding a Cas effector protein, and an inhibitor of the microhomology-mediated end joining (MMEJ) pathway. In some embodiments, the composition further comprises an inhibitor of the non-homologous end joining (NHEJ) pathway.

In some embodiments, the composition further comprises a polynucleotide comprising at least one RNA guide sequence, a Cas-binding region, a DNA template sequence, or combinations thereof.

In some embodiments, the Cas effector protein is a Cas9 nuclease, a Cas12a nuclease, or a Cas12f nuclease. In some embodiments the Cas effector protein is a Cas9 nuclease. In some embodiments, the Cas9 nuclease is a Cas9 nuclease fused to a reverse transcriptase, a Cas9 nuclease fused to a DNA polymerase, a Cas9 fused to DN1S, a Cas9 nickase, a Cas9 fused to a Geminin degron domain, or a Cas9 nuclease fused to CTIP.

In some embodiments, the vector encoding the Cas effector protein is a viral vector.

In some embodiments, the polynucleotide comprising at least one RNA guide sequence, a Cas-binding region, a DNA template sequence, or combinations thereof, is encoded on a vector. In some embodiments the vector is a viral vector.

In some embodiments, the Cas effector protein and the polynucleotide comprising at least one RNA guide sequence, a Cas-binding region, a DNA template sequence, or combinations thereof, are in the form of a ribonucleoprotein (RNP).

In some embodiments, the composition further comprises a pharmaceutically acceptable carrier, diluent, or excipient.

In some embodiments, the present disclosure relates to a kit comprising a Cas effector protein or a vector encoding a Cas effector protein and an inhibitor of the microhomology-mediated end joining (MMEJ) pathway.

In some embodiments, the kit further comprises an inhibitor of the non-homologous end-joining (NHEJ) pathway.

In some embodiments, the kit further comprises a polynucleotide comprising at least one RNA guide sequence, a Cas-binding region, a DNA template sequence, or combinations thereof.

In some embodiments, the Cas effector protein is a Cas9 nuclease, a Cas12a nuclease, or a Cas12f nuclease. In some embodiments, the Cas effector protein is a Cas9 nuclease. In some embodiments, the Cas9 nuclease is a Cas9 nuclease fused to a reverse transcriptase, a Cas9 fused to a DNA polymerase, a Cas9 fused to DN1S, a Cas9 nickase, a Cas9 fused to a Geminin degron domain, or a Cas9 nuclease fused to CTIP.

In some embodiments, the polynucleotide comprising at least one RNA guide sequence, a Cas-binding region, a DNA template sequence, or combinations thereof, is encoded on a vector. In some embodiments, the vector is a viral vector.

In some embodiments, the Cas effector protein and the polynucleotide comprising at least one RNA guide sequence, a Cas-binding region, a DNA template sequence, or combinations thereof, are in the form of a ribonucleoprotein (RNP).

### BRIEF DESCRIPTION OF THE DRAWINGS

FIG. 1 is a schematic showing manipulation of DNA repair with small molecule inhibitors. In this schematic, components of a CRISPR/Cas genome editing system provide double stranded breaks (DSB) at specific sequences. The DSB can be repaired by the imprecise and error-prone microhomology-mediated end joining (MMEJ) or non-homologous end joining (NHEJ) pathways, or alternatively, by the more precise homology directed repair (HDR) pathway.

FIG. 2A-2B illustrate an exemplary method described in embodiments herein. FIG. 2A shows an example in which cells are pre-treated for 3 hours with pharmacological inhibitors of POL Q/DNA polymerase θ (PolQi) and/or DNA-dependent protein kinase (DNA-PKi). A CRISPR/Cas gene editing system is then added to the cells. After 60 hours, genomic DNA is isolated from the cells and deep-targeted sequencing is performed. The results of the sequencing are then analyzed by Rational InDel Meta-Analysis (RIMA) in order to determine the frequency of MMEJ and NHEJ repairs. FIG. 2B shows a graphical representation of the RIMA results, where deletions associated with microhomologies are visualized according to the bars shown in the figure.

FIG. 3 shows the chemical structures of representative POL Q/DNA polymerase θ inhibitors.

FIG. 4 shows that inhibiting the MMEJ and NHEJ pathways results in increased HDR repair of DSB. HEK293T cells were treated with the DNA-PK inhibitor AZD7648 (1 μM) alone and in combination with the indicated Pol Q inhibitors, followed by CRISPR/Cas9-mediated gene targeting. Addition of a DNA-PK inhibitor and Pol Q inhibitors decreased DNA repair by MMEJ and NHEJ, while increasing HDR-mediated DNA repair, as assessed by the percentage of precise DNA repair.

FIG. 5 shows the effect of MMEJ and NHEJ pathway inhibition on CRISPR/Cas editing efficiency as described in Example 1.

FIG. 6 shows the effect of MMEJ and NHEJ pathway inhibition on CRISPR/Cas-mediated gene knock-in efficiency as measured by mutated reads as described in Example 2.

FIG. 7 shows the effect of MMEJ and NHEJ pathway inhibition on CRISPR/Cas-mediated gene knock-in efficiency as measured by mapped reads as described in Example 2.

FIG. 8 shows the effect of Pol Q inhibition on MMEJ in mutated reads as described in Example 3.

FIG. 9 shows the effect of Pol Q inhibition on MMEJ in mapped reads as described in Example 3. HEK293T cells were treated with the DNA-PK inhibitor AZD7648 (1 μM) alone and in combination with the indicated Pol Q inhibitors, followed by CRISPR/Cas9-mediated gene knock-in. Addition of Pol Q inhibitors resulted in a dose-dependent decrease in MMEJ in mapped reads.

FIG. 10 shows the effect of MMEJ and NHEJ pathway inhibition on cell confluency as described in Example 4.

FIG. 11 shows the effect of MMEJ and NHEJ pathway inhibition on transfection efficiency as described in Example 4.

FIG. 12 shows that inhibiting the MMEJ and NHEJ pathways results in increased HDR repair of DSB in induced Pluripotent Stem Cells (iPSC). Cas9-inducible iPSCs were treated with the DNA-PK inhibitor AZD7648 (1 μM) and/or the indicated Pol Q inhibitors, followed by induction of Cas9-mediated gene targeting. Addition of a DNA-PK inhibitor and Pol Q inhibitors decreased DNA repair by MMEJ and NHEJ, while increasing HDR-mediated DNA repair, as assessed by the percentage of precise DNA repair at 3 separate target sites.

FIG. 13 shows the effect of Pol Q inhibition on single-stranded template repair (SSTR)-mediated knock-in efficiency in Cas9-inducible iPSCs. Cas9-inducible iPSCs were treated with the DNA-PK inhibitor ZAD7648 at 1 μM and/or the indicated Pol Q inhibitors, followed by induction of Cas9-mediated gene knock-in at three separate target sites. Addition of a DNA-PK inhibitor and/or a Pol Q inhibitor increased SSTR-mediated knock-in at all three target sites.

FIG. 14A-14C show the effect of inhibiting the MMEJ and NHEJ pathways on gene editing in primary human T cells. Green fluorescent protein (GFP) was inserted via knock-in into primary human T cells which were transfected with Cas9 in the form of a ribonucleoprotein (RNP) which targets TRAC. The cells were treated with the DNA-PK inhibitor AZD7648 at 1 μM, alone and in combination with the indicated Pol Q inhibitors. (A) shows the effect of NHEJ and/or MMEJ pathway inhibition on cell viability. (B) shows the effect of NHEJ and/or MMEJ pathway inhibition on cell number. (C) shows the effect of NHEJ and/or MMEJ pathway inhibition on GFP knock-in efficiency.

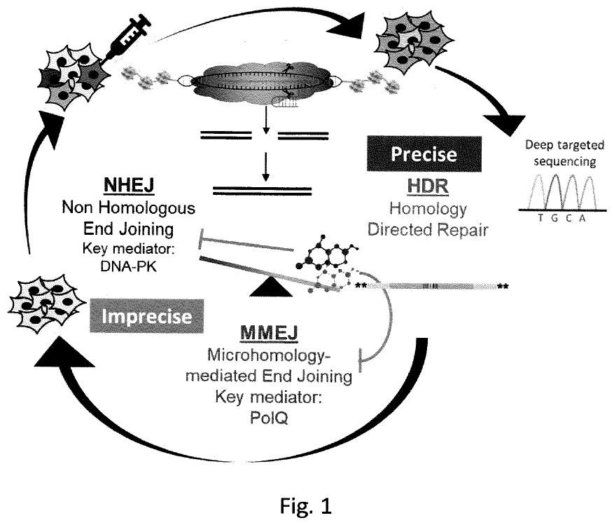

### DETAILED DESCRIPTION OF THE INVENTION

The present disclosure relates to methods of improving CRISPR/Cas-mediated gene insertion (i.e. gene “knock-in”) in eukaryotic cells, compositions for improved CRISPR/Cas-mediated insertion, and kits for improved CRISPR/Cas-mediated gene insertion. In general a CRISPR system, e.g., a CRISPR/Cas system, includes elements that promote the formation of a CRISPR complex, such as a guide polynucleotide and a Cas protein, at the site of a target polynucleotide, e.g., a target DNA sequence. In naturally-occurring CRISPR systems (e.g., the bacterial immunity CRISPR/Cas9 system), foreign DNA is incorporated into CRISPR arrays, which then produce CRISPR-RNAs (crRNA). The crRNA includes RNA guide sequence regions complementary to the foreign DNA site and hybridizes with trans-activating CRISPR-RNA (tracrRNA), which is also encoded by the CRISPR system. The tracrRNA forms secondary structures, e.g., stem loops, and is capable of binding to Cas9 protein. The crRNA/tracrRNA hybrid associates with Cas9, and the crRNA/tracrRNA/Cas9 complex recognizes and cleaves foreign DNA bearing the protospacer sequences, thereby conferring immunity against the invading virus or plasmid. CRISPR/Cas systems are further described in, e.g., Jinek et al., Science 337 (6096): 816-821 (2012); Cong et al., Science 339 (6121): 819-823 (2013); Mali et al., Science 339 (6121): 823-826 (2013); and Sander et al., Nat Biotechnol 32:347-355 (2014).

CRISPR/Cas systems have been engineered to introduce insertions into a target polynucleotide, also known as targeted insertions. Typically, the guide polynucleotide is designed such that the Cas protein generates a double-stranded cleavage at the target polynucleotide, and a separate donor template comprising the sequence of interest is inserted into the cleaved target polynucleotide by cellular DNA repair mechanisms, e.g., non-homologous end joining (NHEJ) or homology directed repair (HDR). The efficiency of insertion is dependent on several factors, including transfection ratio of the donor template, Cas protein, and guide polynucleotide; sequence and size of the donor template; and type of DNA repair mechanism triggered. For example, HDR provides high-fidelity DNA repair but has low insertion frequency, while NHEJ has higher insertion frequency but may also introduce mutations into the target DNA.

In some embodiments, the present disclosure provides compositions, polynucleotides, and/or fusion proteins for improved targeted insertion methods. In some embodiments, the compositions, polynucleotides, and/or fusion proteins of the present disclosure provide higher precision of inserting a sequence of interest. In some embodiments, the compositions, polynucleotides, and fusion proteins of the present disclosure provide higher efficiency of inserting a sequence of interest.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps. It is contemplated that any embodiment discussed in this specification can be implemented with respect to any protein, compositions, polynucleotides, vectors, cells, methods, and/or kits of the present disclosure. Furthermore, compositions, polynucleotides, vectors, cells, and/or kits of the present disclosure can be used to achieve methods and proteins of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

A “nucleic acid,” “nucleic acid molecule,” “nucleotide,” “nucleotide sequence,” “oligonucleotide,” or “polynucleotide” means a polymeric compound including covalently linked nucleotides. The term “nucleic acid” includes ribonucleic acid (RNA) or deoxyribonucleic acid (DNA) both of which may be single- or double-stranded. The polynucleotide may comprise naturally-occurring nucleobases (e.g., guanine, adenine, cytosine, thymine, and uracil), modified nucleobases (e.g., hypoxanthine, xanthine, 7-methylguanine, dihydrouracil, 5-methylcytosine, 5-hydroxymethylcytosine), and/or artificial nucleobases (e.g., isoguanine or isocytosine). Nucleic acids are transcribed from a 5′ end to a 3′ end. In some embodiments, the disclosure provides a polynucleotide comprising RNA and DNA nucleotides. Methods of producing a polynucleotide comprising both RNA and DNA nucleotides are known in the art and include, e.g., ligation or oligonucleotide synthesis methods. In some embodiments, the disclosure provides a polynucleotide capable of forming a complex with a Cas nuclease or Cas nickase as described herein. In some embodiments, the disclosure provides a polynucleotide encoding any one of the proteins disclosed herein, e.g., a Cas nuclease or Cas nickase.

A “gene” refers to an assembly of nucleotides that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

A nucleic acid molecule is “hybridizable” or “hybridized” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are known and exemplified in Sambrook et al., Molecular Cloning: A Laboratory Manual, Second Edition, Cold Spring Harbor Laboratory Press, Cold Spring Harbor (1989), particularly Chapter 11 and Table 11.1 therein. The conditions of temperature and ionic strength determine the stringency of the hybridization. The stringency of the hybridization conditions can be selected to provide selective formation or maintenance of a desired hybridization product of two complementary polynucleotides, in the presence of other potentially cross-reacting or interfering polynucleotides. Stringent conditions are sequence-dependent; typically, longer complementary sequences specifically hybridize at higher temperatures than shorter complementary sequences. Generally, stringent hybridization conditions are between about 5° C. to about 10° C. lower than the thermal melting point (Tm) (i.e., the temperature at which 50% of the sequences hybridize to a substantially complementary sequence) for a specific polynucleotide at a defined ionic strength, concentration of chemical denaturants, pH, and concentration of the hybridization partners. Generally, nucleotide sequences having a higher percentage of G and C bases hybridize under more stringent conditions than nucleotide sequences having a lower percentage of G and C bases. Generally, stringency can be increased by increasing temperature, increasing pH, decreasing ionic strength, and/or increasing the concentration of chemical nucleic acid denaturants (such as formamide, dimethylformamide, dimethylsulfoxide, ethylene glycol, propylene glycol and ethylene carbonate). Stringent hybridization conditions typically include salt concentrations or ionic strength of less than about 1 M, 500 mM, 200 mM, 100 mM or 50 mM; hybridization temperatures above about 20° C., 30° C., 40° C., 60° C. or 80° C.; and chemical denaturant concentrations above about 10%, 20%, 30% 40% or 50%. Because many factors can affect the stringency of hybridization, the combination of parameters may be more significant than the absolute value of any parameter alone.

The term “complementary” is used to describe the relationship between nucleotide bases that are capable of hybridizing to one another. For example, with respect to DNA, adenosine is complementary to thymine and cytosine is complementary to guanine. When two nucleic acids are “complementary,” it is meant that a first nucleic acid or one or more regions thereof is capable of hydrogen bonding with a second nucleic acid or one or more regions thereof. Complementary nucleic acids need not have complementarity at each nucleotide and may include one or more nucleotide mismatches, i.e., points at which hydrogen bonding does not occur. For example, complementary oligonucleotides can have at least 80%, at least 85%, at least 90%, at least 95%, at least 96%, at least 97%, at least 98%, or at least 99% of nucleotides hydrogen bond. By contrast, “fully complementary” or “100% complementary” in reference to oligonucleotides means that each nucleotide hydrogen bonds without any nucleotide mismatches.

The term “homologous recombination” refers to the insertion of an exogenous polynucleotide (e.g., DNA) into another nucleic acid (e.g., DNA) molecule, e.g., insertion of a vector, polynucleotide fragment or gene in a chromosome. In some cases, the exogenous polynucleotide targets a specific chromosomal site for homologous recombination. For specific homologous recombination, the exogenous polynucleotide typically contains sufficiently long regions of homology to sequences of the chromosome to allow complementary binding and incorporation of the exogenous polynucleotide into the chromosome. Longer regions of homology and greater degrees of sequence similarity may increase the efficiency of homologous recombination. In some embodiments, the polynucleotides or compositions described herein facilitate homologous recombination by generating breaks, e.g., double-stranded breaks in a nucleic acid sequence.

The term “homology-directed repair” or “HDR” refers to a mechanism of repairing double-stranded breaks in DNA using a template nucleic acid sequence. The most common form of HDR is homologous recombination. In HDR, a double-stranded break is repaired by a process involving resection of the 5′ ended DNA strand at the break to create a 3′ overhang, which serves as both a substrate for proteins required for strand invasion and as a primer for DNA repair synthesis. The invasive strand then displaces one strand of a double-stranded DNA template sequence which comprises homologous sequences and pair with the other strand, resulting in the formation of hybrid DNA known as the displacement loop. These recombination intermediates are then resolved to complete the DNA repair process.

The term “single-strand template repair” or “SSTR” refers to another mechanism of repairing double-stranded breaks in DNA using a template nucleic acid sequence. In contrast to HDR, SSTR utilizes a single-stranded template nucleic acid sequence for double-strand DNA break repair.

The term “non-homologous end joining pathway” or “NHEJ pathway” refers to another mechanism of repairing double-stranded breaks in DNA. In NHEJ, a Ku80/70 heterodimer recognizes and binds to blunt ends formed by the double-stranded break, where the resulting complex activates the activity of DNA-PK. Activation of DNA-PK recruits Artemis nuclease, DNA polymerases, and DNA ligases to ultimately repair the double-stranded break. NHEJ differs from HDR and homologous recombination that that it does not require a homologous template sequence for repair.

The term “microhomology-mediated end joining pathway” or “MMEJ pathway” refers to another mechanism for repairing double-stranded breaks in DNA. MMEJ is similar to NHEJ in that a homologous template sequence is not utilized for double-stranded break repair. However, MMEJ is distinguished from other repair mechanisms by its utilization of microhomologous sequences to align broken DNA strands. MMEJ does not rely on Ku protein or DNA-PK, but DNA polymerase θ (Pol Q) has been shown to be required for MMEJ. MMEJ is also known as “alternative end-joining,” or “alternative nonhomologous end-joining” or “Alt-NHEJ.”

As used herein, the term “operably linked” means that a polynucleotide of interest, e.g., the polynucleotide encoding a nuclease, is linked to the regulatory element in a manner that allows for expression of the polynucleotide. Regulatory elements can be cis-regulatory elements or trans-regulatory elements. Regulatory elements include, for example, promoters, enhancers, terminators, 5′ and 3′ UTRs, insulators, silencers, operators, and the like. In some embodiments, the regulatory element is a promoter. In some embodiments, a polynucleotide expressing a protein of interest is operably linked to a promoter on an expression vector.

As used herein, “promoter,” “promoter sequence,” or “promoter region” refers to a DNA regulatory region or polynucleotide capable of binding RNA polymerase and involved in initiating transcription of a downstream coding or non-coding sequence. In some embodiments, the promoter sequence includes the transcription initiation site and extends upstream to include the minimum number of bases or elements used to initiate transcription at levels detectable above background. In some embodiments, the promoter sequence includes a transcription initiation site, as well as protein binding domains responsible for the binding of RNA polymerase. Eukaryotic promoters typically contain “TATA” boxes and “CAT” boxes. Various promoters, including inducible promoters, may be used to drive expression of the various vectors of the present disclosure.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. In some embodiments, the vector is an episomal vector, which is removed/lost from a population of cells after a number of cellular generations, e.g., by asymmetric partitioning. The term “vector” includes both viral and non-viral means for introducing the nucleic acid into a cell in vitro, ex vivo, or in vivo. A large number of vectors known in the art may be used to manipulate nucleic acids, incorporate response elements and promoters into genes, etc. A vector may include one or more regulatory regions, and/or selectable markers useful in selecting, measuring, and monitoring nucleic acid transfer results (transfer to which tissues, duration of expression, etc.).

Possible vectors include, for example, plasmids or modified viruses including, for example, bacteriophages such as lambda derivatives, or plasmids such as PBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified, or any site may be produced by ligating polynucleotides (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include, but are not limited, to retrovirus, lentivirus, adenovirus, adeno-associated virus, pox, baculovirus, vaccinia, herpes simplex, Epstein-Barr, adenovirus, geminivirus, and caulimovirus vectors. In some embodiments, a viral vector is utilized to provide the polynucleotides described herein. In some embodiments, a viral vector is utilized to provide a polynucleotide coding for a protein described herein.

Vectors may be introduced into the desired host cells by known methods, including, but not limited to, transfection, transduction, cell fusion, and lipofection. Vectors can include various regulatory elements including promoters. In some embodiments, vector designs can be based on constructs designed by Mali et al., Nat Methods 10:957-63 (2013).

Methods known in the art may be used to propagate polynucleotides and/or vectors provided herein. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors.

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of polynucleotides have been joined or recombined into a unique construction which is capable of introducing a promoter fragment and DNA sequence for a selected gene product along with appropriate 3′ untranslated sequence into a cell. In some embodiments, a plasmid is utilized to provide the polynucleotides described herein. In some embodiments, a plasmid is utilized to provide a polynucleotide coding for a protein described herein.

The term “transfection” as used herein means the introduction of an exogenous nucleic acid molecule, including a vector, into a cell. Transfection methods, e.g., for components of the CRISPR/Cas compositions described herein, are known to one of ordinary skill in the art. A “transfected” cell includes an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to herein as “recombinant,” “transformed,” or “transgenic” organisms. In some embodiments, the present disclosure provides a host cell comprising any of the vectors described herein, e.g., a vector comprising a Cas polynucleotide, a vector comprising the polynucleotide of interest, or a vector comprising a polynucleotide comprising an RNA guide sequence, a CAS-binding region, a DNA Template sequence or combinations thereof.

The term “host cell” refers to a cell into which a recombinant expression vector has been introduced, or “host cell” may also refer to the progeny of such a cell. Because modifications may occur in succeeding generations, for example, due to mutation or environmental influences, the progeny may not be identical to the parent cell, but are still included within the scope of the term “host cell.”

The terms “peptide,” “polypeptide,” and “protein” are used interchangeably herein, and refer to a polymeric form of amino acids of any length, which can include coded and non-coded amino acids, non-naturally occurring amino acids, chemically or biochemically modified or derivatized amino acids, peptides and polypeptides having modified peptide backbones, and circular/cyclic peptides and polypeptides.

The start of the protein or polypeptide is known as the “N-terminus” (and also referred to as the amino-terminus, NH₂-terminus, N-terminal end or amine-terminus), referring to the free amine (—NH₂) group of the first amino acid residue of the protein or polypeptide. The end of the protein or polypeptide is known as the “C-terminus” (and also referred to as the carboxy-terminus, carboxyl-terminus, C-terminal end, or COOH-terminus), referring to the free carboxyl group (—COOH) of the last amino acid residue of the protein or polypeptide.

An “amino acid” as used herein refers to a compound including both a carboxyl (—COOH) and amino (—NH₂) group. “Amino acid” refers to both natural and unnatural, i.e., synthetic, amino acids. Natural amino acids, with their three-letter and single-letter abbreviations, include: alanine (Ala; A); arginine (Arg, R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V). Unnatural or synthetic amino acids include a side chain that is distinct from the natural amino acids provided above and may include, e.g., fluorophores, post-translational modifications, metal ion chelators, photocaged and photocross-linking moieties, uniquely reactive functional groups, and NMR, IR, and x-ray crystallographic probes. Exemplary unnatural or synthetic amino acids are provided in, e.g., Mitra et al., Mater Methods 3:204 (2013) and Wals et al., Front Chem 2:15 (2014). Unnatural amino acids may also include naturally-occurring compounds that are not typically incorporated into a protein or polypeptide, such as, e.g., citrulline (Cit), selenocysteine (Sec), and pyrrolysine (Pyl).

An “amino acid substitution” refers to a polypeptide or protein including one or more substitutions of wild-type or naturally occurring amino acid with a different amino acid relative to the wild-type or naturally occurring amino acid at that amino acid residue. The substituted amino acid may be a synthetic or naturally occurring amino acid. In some embodiments, the substituted amino acid is a naturally occurring amino acid selected from the group consisting of: A, R, N, D, C, Q, E, G, H, I, L, K, M, F, P, S, T, W, Y, and V. In some embodiments, the substituted amino acid is an unnaturally or synthetic amino acid. Substitution mutants may be described using an abbreviated system. For example, a substitution mutation in which the fifth (5^{th}) amino acid residue is substituted may be abbreviated as “X5Y,” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide, protein, peptide, or nucleic acid is a molecule that has been removed from its natural environment. It is also understood that “isolated” polypeptides, proteins, peptides, or nucleic acids may be formulated with excipients such as diluents or adjuvants and still be considered isolated. As used herein, “isolated” does not necessarily imply any particular level purity of the polypeptide, protein, peptide, or nucleic acid.

The term “recombinant” when used in reference to a nucleic acid molecule, peptide, polypeptide, or protein means of, or resulting from, a new combination of genetic material that is not known to exist in nature. A recombinant molecule can be produced by any of the techniques available in the field of recombinant technology, including, but not limited to, polymerase chain reaction (PCR), gene splicing (e.g., using restriction endonucleases), and solid-phase synthesis of nucleic acid molecules, peptides, or proteins.

The term “exogenous” means that the referenced molecule or activity introduced into the host cell. The molecule can be introduced, for example, by introduction of an encoding nucleic acid into the host genetic material, such as by integration into a host chromosome or as non-chromosomal genetic material, e.g., a plasmid. An “exogenous” protein can be introduced into a host cell via an “exogenous” nucleic acid encoding the protein. The term “endogenous” refers to a referenced molecule or activity that is naturally present in the host cell. An “endogenous” protein is expressed by a nucleic acid contained within the host cell. The term “heterologous” refers to a molecule or activity derived from a source other than the referenced organism/species, whereas “homologous” refers to a molecule or activity derived from the host organism/species. Accordingly, exogenous expression of an encoding nucleic acid can utilize either or both of a heterologous or homologous encoding nucleic acid.

The term “domain” when used in reference to a polypeptide or protein means a distinct functional and/or structural unit in a protein. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function.

The term “motif,” when used in reference to a polypeptide or protein, generally refers to a set of conserved amino acid residues, typically shorter than 20 amino acids in length, that may be important for protein function. Specific sequence motifs may mediate a common function, such as protein-binding or targeting to a particular subcellular location, in a variety of proteins. Examples of motifs include, but are not limited to, nuclear localization signals, microbody targeting motifs, motifs that prevent or facilitate secretion, and motifs that facilitate protein recognition and binding. Motif databases and/or motif searching tools are known in the field and include, for example, PROSITE, PFAM, PRINTS, and MiniMotif Miner.

An “engineered” protein, as used herein, means a protein that includes one or more modifications in a protein to achieve a desired property. Exemplary modifications include, but are not limited to, insertion, deletion, substitution, and/or fusion with another domain or protein. A “fusion protein” (also termed “chimeric protein”) is a protein comprising at least two domains, typically coded by two separate genes, that have been joined such that they are transcribed and translated as a single unit, thereby producing a single polypeptide having the functional properties of each of the domains. Engineered proteins of the present disclosure include Cas nucleases, Cas nickases, and fusions of Cas proteins with a DNA polymerase, DNA ligase, and/or DNA polymerase-binding protein.

In some embodiments, engineered protein is generated from a wild-type protein. As used herein, a “wild-type” protein or nucleic acid is a naturally-occurring, unmodified protein or nucleic acid. For example, a wild-type Cas9 protein can be isolated from the organism Streptococcus pyogenes. Wild-type can be contrasted with “mutant,” which includes one or more modifications in the amino acid and/or nucleotide sequence of the protein or nucleic acid. In some embodiments, an engineered protein can have substantially the same activity as a wild-type protein, e.g., greater than about 80%, greater than about 85%, greater than about 90%, greater than about 95%, or greater than about 99% of the activity as a wild-type protein. In some embodiments, the Cas nuclease of a fusion protein described herein has substantially the same activity as a wild-type Cas nuclease.

In some embodiments, an engineered protein, e.g., a Cas9 protein, can have substantially the same amino acid sequence as a wild-type protein, e.g., greater than about 80%, greater than about 85%, greater than about 90%, greater than about 95%, or greater than about 99% identify as a wild-type protein. As used herein, the terms “sequence similarity” or “% similarity” refers to the degree of identity or correspondence between nucleic acid sequences or amino acid sequences. In the context of polynucleotides, “sequence similarity” may refer to nucleic acid sequences where changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the encoded polypeptide.

Moreover, the skilled artisan recognizes that similar polynucleotides encompassed by the present disclosure are also defined by their ability to hybridize, under stringent conditions, with the sequences exemplified herein. Similar polynucleotides of the present disclosure are about 70%, at least about 70%, about 75%, at least about 75%, about 80%, at least about 80%, about 85%, at least about 85%, about 90%, at least about 90%, about 95%, at least about 95%, about 99%, at least about 99%, or about 100% identical to the polynucleotides disclosed herein.

In the context of polypeptides, “sequence similarity” refers to two or more polypeptides where greater than about 40% of the amino acids are identical, or greater than about 60% of the amino acids are functionally identical. “Functionally identical” or “functionally similar” amino acids have chemically similar side chains. For example, amino acids can be grouped in the following manner according to functional similarity: (i) positively-charged side chains: Arg, His, Lys; (ii) negatively-charged side chains: Asp, Glu; (iii) polar, uncharged side chains: Ser, Thr, Asn, Gln; (iv) hydrophobic side chains: Ala, Val, Ile, Leu, Met, Phe, Tyr, Trp; and (v) others: Cys, Gly, Pro.

In some embodiments, similar polypeptides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acids. In some embodiments, similar polypeptides of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

Sequence similarity can be determined by sequence alignment using methods known in the field, such as, for example, BLAST, MUSCLE, Clustal (including ClustalW and ClustalX), and T-Coffee (including variants such as, for example, M-Coffee, R-Coffee, and Expresso).

Percent identity of polynucleotides or polypeptides can be determined when the polynucleotide or polypeptide sequences are aligned over a specified comparison window. In some embodiments, only specific portions of two or more sequences are aligned to determine sequence identity. In some embodiments, only specific domains of two or more sequences are aligned to determine sequence similarity. A comparison window can be a segment of at least 10 to over 1000 residues, at least 20 to about 1000 residues, or at least 50 to 500 residues in which the sequences can be aligned and compared. Methods of alignment for determination of sequence identity are well-known and can be performed using publicly available databases such as BLAST. For example, in some embodiments, “percent identity” of two amino acid sequences is determined using the algorithm of Karlin and Altschul, Proc Nat Acad Sci USA 87:2264-2268 (1990), modified as in Karlin and Altschul, Proc Nat Acad Sci USA 90:5873-5877 (1993). Such algorithms are incorporated into BLAST programs, e.g., BLAST+ or the NBLAST and XBLAST programs described in Altschul et al., J Mol Biol, 215:403-410 (1990). BLAST protein searches can be performed with programs such as, e.g., the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Res 25 (17): 3389-3402 (1997). When utilizing BLAST and Gapped BLAST programs, the default parameters of the respective programs (e.g., XBLAST and NBLAST) can be used.

In some embodiments, a polypeptide or polynucleotide has 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100% sequence identity with a reference polypeptide or polynucleotide (or a fragment of the reference polypeptide or polynucleotide) provided herein. In some embodiments, a polypeptide or polynucleotide have about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99% or about 100% sequence identity with a reference polypeptide or polynucleotide (or a fragment of the reference polypeptide or nucleic acid molecule) provided herein.

As used herein, a “complex” refers to a group of two or more associated polynucleotides and/or polypeptides. In the context of complex formation, the terms “associate” or “association” refers to molecules bound to one another through electrostatic, hydrophobic/hydrophilic, and/or hydrogen bonding interaction, without being covalently attached. A molecule that comprises different moieties covalently attached to one another is known. In some embodiments, a complex is formed when all the components of the complex are present together, i.e., a self-assembling complex. In some embodiments, a complex is formed through chemical interactions between different components of the complex such as, for example, hydrogen-bonding. In some embodiments, the polynucleotides provided herein form a complex with the proteins provided herein through secondary structure recognition of the polynucleotide by the protein. In some embodiments, the Cas-binding region of the polynucleotides provided herein comprise a secondary structure recognized by a Cas nuclease, Cas nickase, or fusion protein provided herein.

### Cas Proteins

As used herein, a “Cas effector protein,” also referred herein as “Cas protein” encompasses both Cas nucleases and Cas nickases. Cas effector proteins are part of the CRISPR/Cas system described herein. CRISPR/Cas systems, which include a Cas effector protein and a polynucleotide (also referred to as a “guide polynucleotide”), can be utilized for site-specific genome modifications. In some embodiments, the CRISPR/Cas system comprises a Cas effector protein and a guide polynucleotide comprising a Cas-binding region (which binds and/or activates the Cas protein) and a guide sequence (which hybridizes to a target sequence), where the Cas effector protein and the guide polynucleotide form a complex as described herein. In some embodiments, the CRISPR/Cas system comprises a Cas effector protein, a first polynucleotide comprising a guide sequence, and a second polynucleotide comprising a Cas-binding region, where the first and second polynucleotides hybridize to each other and form a complex with the Cas effector protein.

CRISPR/Cas systems can be classified as Types I to VI based on the Cas effector protein in the system. For example, Cas9 is found in Type II systems, and Cas12 is found in Type V systems. Each Type can be further divided into subtypes. For example, Type II can include subtypes II-A, II-B, and II-C, and Type V can include subtypes V-A and V-B. Classification of CRISPR/Cas systems and Cas nucleases is further discussed in, e.g., Makarova et al., Methods Mol Biol 1311:47-75 (2015); Makarova et al., The CRISPR Journal October 2018; 325-336; and Koonin et al., Phil Trans R Soc B 374:20180087 (2018). Cas nucleases described herein can encompass any Type or variant, unless otherwise specified.

In some embodiments, the Cas effector protein is a Cas nuclease. In general, a Cas effector nuclease is capable of generating a double-stranded polynucleotide cleavage, e.g., a double-stranded DNA cleavage. In general, a Cas nuclease can include one or more nuclease domains, such as RuvC and HNH, and can cleave double-stranded DNA. In some embodiments, a Cas nuclease comprises a RuvC domain and an HNH domain, each of which cleaves one strand of double-stranded DNA. In some embodiments, the Cas nuclease generates blunt ends. In some embodiments, the RuvC and HNH of a Cas nuclease cleaves each DNA strand at the same position, thereby generating blunt ends. In some embodiments, the Cas nuclease generates cohesive ends. In some embodiments, the RuvC and HNH of a Cas nuclease cleaves each DNA strand at different positions (i.e., cut at an “offset”), thereby generating cohesive ends. As used herein, the terms “cohesive ends,” “staggered ends,” or “sticky ends” refer to a nucleic acid fragment with strands of unequal length. In contrast to “blunt ends,” cohesive ends are produced by a staggered cut on a double-stranded nucleic acid (e.g., DNA). A sticky or cohesive end has protruding singles strands with unpaired nucleotides, or “overhangs,” e.g., a 3′ or a 5′ overhang.

In some embodiments, the Cas nuclease is a Cas9 nuclease. Exemplary Cas9 nucleases include, but are not limited to, the Cas9 from Streptococcus pyogenes, Streptococcus thermophilus, Streptococcus mutans, Listeria innocua, Neisseria meningitidis, Staphylococcus aureus, Klebisella pneumoniae, and numerous other bacteria. Further exemplary Cas9 nucleases are described in, e.g., U.S. Pat. Nos. 8,771,945; 9,023,649; 10,000,772; 10,407,697; and US 2014/0068797. In some embodiments, the Cas9 nuclease is from S. pyogenes (SpCas9).

In some embodiments, the Cas9 nuclease comprises the sequence disclosed in UniProt ID G3ECR1 (SEQ ID NO: 1), UniProt ID Q99ZW2 (SEQ ID NO: 2), or UniProt ID J7RUA5 (SEQ ID NO: 3). In some embodiments, the Cas9 comprises a polypeptide sequence having at least 70%, at least 75%, at least 80%, at least 85%, at least 90%, at least 95%, at least 96%, at least 97%, at least 98%, at least 99%, or about 100% sequence identity to any of SEQ ID NOs: 1-3. In some embodiments, the disclosure provides for a polynucleotide which encodes a polypeptide having at least 70%, at least 75%, at least 80%, at least 85%, at least 90%, at least 95%, at least 96%, at least 97%, at least 98%, at least 99%, or about 100% sequence identity to any of SEQ ID NOs: 1-3. In some embodiments, the Cas9 is encoded by a polynucleotide which has been codon optimized for expression in a host cell.

In some embodiments, the Cas9 nuclease is a Type IIB Cas9 nuclease. In general, Type IIB Cas9 proteins are capable of generating cohesive ends, as described herein. Exemplary Type IIB Cas9 proteins include, but are not limited to, the Cas9 protein from Legionella pneumophila, Francisella novicida, Parasutterella excrementihominis, Sutterella wadsworthensis, Wolinella succinogenes, and numerous other bacteria. Further Type IIB Cas9 proteins are described in, e.g., WO 2019/099943.

In some embodiments, the Cas effector protein is a Cas12 nuclease. In some embodiments, the Cas nuclease is a Cas12a nuclease (formerly known as “Cpf1” or “C2c1”). In some embodiments, the Cas nuclease is a Cas12f nuclease. Cas12f nuclease is also known in the art as Cas14 (Makarova et al, Nature Rev. Microbiol., 2019, 18:67-83). In some embodiments, the Cas nuclease is a Cas14 nuclease. Cas12 nucleases are generally smaller than Cas9 nucleases and can typically generate cohesive ends. Exemplary Cas12 proteins include, but are not limited to, the Cas12 protein from Francisella novicida, Acidaminococcus sp., Lachnospiraceae sp., Prevotella sp., and numerous other bacteria. Further Cas12 nuclease are described in, e.g., U.S. Pat. No. 9,580,701; US 2016/0208243; Zetsche et al., Cell 163 (3): 759-771 (2015); and Chen et al., Science 360:436-439 (2018).

In some embodiments, the Cas12 nuclease comprises the sequence disclosed by UniProt ID A0Q7Q2 SEQ ID NO: 4), UniProt ID U2UMQ6 (SEQ ID NO: 5), or UniProt ID T0D7A2 (SEQ ID NO: 6). In some embodiments, the Cas12 has at least 70%, at least 75%, at least 80%, at least 85%, at least 90%, at least 95%, at least 96%, at least 97%, at least 98%, at least 99%, or about 100% sequence identity to any of SEQ ID NOs: 4-6. In some embodiments, the disclosure provides for a polynucleotide which encodes a polypeptide having at least 70%, at least 75%, at least 80%, at least 85%, at least 90%, at least 95%, at least 96%, at least 97%, at least 98%, at least 99%, or about 100% sequence identity to the polypeptide of any of SEQ ID NOs: 4-6. In some embodiments, the Cas12 is encoded by a polynucleotide which has been codon optimized for expression in a host cell.

In some embodiments, the Cas effector protein is a Cas nickase. A nickase, which generates a single-stranded cleavage on a double-stranded polynucleotide (e.g., DNA), is distinguished from a nuclease, which cleaves both strands of a double-stranded polynucleotide (e.g., DNA). As discussed herein, a wild-type Cas nuclease typically comprises two catalytic nuclease domains, RuvC and HNH, and each nuclease domain is responsible for cleavage of one strand of double-stranded DNA. Thus, in some embodiments, a Cas nickase comprises an amino acid mutation in a catalytic domain relative to a Cas nuclease. Cas nickases are further described in, e.g., Cho et al., Genome Res 24:132-141 (2013); Ran et al., Cell 154:1380-1389 (2013); and Mali et al., Nat Biotechnol 31:833-838 (2013).

In some embodiments, the Cas nickase is a Cas9 nickase. In some embodiments, the Cas nickase is a Cas12a nickase. In some embodiments, the Cas nickase is a Type II-B Cas nickase. In some embodiments, the Cas nickase is produced by providing a mutation in a Cas nuclease. For example, the SpCas9 nickase comprises a D10A mutation or H840A mutation relative to wild-type SpCas9 nuclease. It will be understood by one of ordinary skill in the art that alignment methods such as those described herein can be used to determine the corresponding amino acid residues in other Cas nucleases (e.g., Cas12a or Type II-B Cas nucleases) to produce a Cas nickase.

In some embodiments, the Cas nuclease or Cas nickase of the composition is not fused to a heterologous protein domain. In some embodiments, the Cas nuclease or Cas nickase is not fused to a DNA polymerase, a DNA ligase, or a reverse transcriptase.

In some embodiments, the recombinant Cas effector proteins of the present disclosure are part of a fusion protein including one or more heterologous protein domains (e.g., about or at least about 1, 2, 3, 4, 5, 6, 7, 8, 9, or 10 or more domains in addition to the recombinant Cas effector protein). A Cas fusion protein can include any additional protein sequence, and optionally a linker sequence between any two domains. Examples of protein domains that may be fused to a recombinant Cas9 protein include, without limitation: epitope tags, reporter gene sequences, and protein domains having one or more of the following activities: methylase activity, demethylase activity, transcription activation activity, transcription repression activity, transcription release factor activity, histone modification activity, RNA cleavage activity, and nucleic acid binding activity. Non-limiting examples of epitope tags include: histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), autofluorescent proteins including blue fluorescent protein (BFP), and mCherry. In some embodiments, a recombinant Cas effector protein is fused to a protein or a fragment of a protein that binds DNA molecules or bind other cellular molecules, including but not limited to: maltose binding protein (MBP), S-tag, Lex A DNA binding domain (DBD), GAL4 DNA binding domain, and herpes simplex virus (HSV) BP16 protein. Additional domains that may form part of a fusion protein including a Cas effector protein are described in U.S. Patent Publication 2011/0059502. In some embodiments, a tagged recombinant Cas effector protein is used to identify the location of a target sequence.

In some embodiments, the Cas effector protein is fused to a heterologous protein or protein domain. In some embodiments, the Cas effector protein is fused to a reverse transcriptase. In some embodiments, the Cas effector protein is a Cas9 nuclease fused to a reverse transcriptase. Examples of such Cas9-reverse transcriptase fusions are described in Anzalone et al., Nature, 576:149-157 (2019).

In some embodiments, the Cas effector protein is fused to a DNA polymerase. In some embodiments, the Cas effector protein is a Cas9 nuclease fused to a DNA polymerase.

In some embodiments, the Cas effector protein is fused to a dominant negative 53BP1 (also known as TP53BP1, tumor suppressor p53-binding protein 1). In some embodiments, the Cas effector protein is a Cas9 nuclease fused to a dominant negative 53BP1 protein. In some embodiments, the dominant negative 53BP1 protein is DN1S. In some embodiments, the Cas effector protein is a Cas9 nuclease fused to DN1S.

In some embodiments, the Cas effector protein is fused to a Geminin degron domain. IN some embodiments, the Cas effector protein is a Cas9 nuclease fused to a Geminin degron domain. Examples of such proteins are described in Gutschner et al, Cell Reports, 14:1555-1566 (2016).

In some embodiments, the Cas effector protein is fused to a CtIP (C-terminal binding protein 1) protein. In some embodiments, the Cas effector protein is a Cas9 nuclease fused to a CtIP protein.

In some embodiments, a recombinant Cas effector protein may form a component of an inducible system. The inducible nature of the system allows for spatiotemporal control of gene editing or gene expression using a form of energy. The form of energy can include, but is not limited to: electromagnetic radiation, sound energy, chemical energy, and thermal energy. Non-limiting examples of inducible system include: tetracycline inducible promoters (Tet-On or Tet-Off), small molecule two-hybrid transcription activations systems (FKBP, ABA, etc), or light inducible systems (Phytochrome, LOV domains, or cryptochrome). In some embodiments, the Cas effector protein is a part of a Light Inducible Transcriptional Effector (LITE) to direct changes in transcriptional activity in a sequence-specific manner. The components of a light may include a Cas effector protein, a light-responsive cytochrome heterodimer (e.g., from Arabidopsis thaliana), and a transcriptional activation/repression domain. Further examples of inducible DNA binding proteins and methods for their use are provided in International Application Publication Nos. WO 2014/018423 and WO 2014/093635; U.S. Pat. Nos. 8,889,418 and 8,895,308; and U.S. Patent Publication Nos. 2014/0186919, 2014/0242700, 2014/0273234, and 2014/0335620.

TABLE 1

| | |
|---|---|
| SEQ ID NO: 1 | MLFNKCIIISINLDFSNKEKCMTKPYSIGLDIGTNSVGWAVITDNYK |
| | VPSKKMKVLGNTSKKYIKKNLLGVLLFDSGITAEGRRLKRTARRR |
| | YTRRRNRILYLQEIFSTEMATLDDAFFQRLDDSFLVPDDKRDSKYPI |
| | FGNLVEEKVYHDEFPTIYHLRKYLADSTKKADLRLVYLALAHMIK |
| | YRGHFLIEGEFNSKNNDIQKNFQDFLDTYNAIFESDLSLENSKQLEEI |
| | VKDKISKLEKKDRILKLFPGEKNSGIFSEFLKLIVGNQADFRKCFNL |
| | DEKASLHFSKESYDEDLETLLGYIGDDYSDVFLKAKKLYDAILLSG |
| | FLTVTDNETEAPLSSAMIKRYNEHKEDLALLKEYIRNISLKTYNEVF |
| | KDDTKNGYAGYIDGKTNQEDFYVYLKNLLAEFEGADYFLEKIDRE |
| | DFLRKQRTFDNGSIPYQIHLQEMRAILDKQAKFYPFLAKNKERIEKI |
| | LTFRIPYYVGPLARGNSDFAWSIRKRNEKITPWNFEDVIDKESSAEA |
| | FINRMTSFDLYLPEEKVLPKHSLLYETFNVYNELTKVRFIAESMRD |
| | YQFLDSKQKKDIVRLYFKDKRKVTDKDIIEYLHAIYGYDGIELKGIE |
| | KQFNSSLSTYHDLLNIINDKEFLDDSSNEAIIEEIIHTLTIFEDREMIKQ |
| | RLSKFENIFDKSVLKKLSRRHYTGWGKLSAKLINGIRDEKSGNTILD |
| | YLIDDGISNRNFMQLIHDDALSFKKKIQKAQIIGDEDKGNIKEVVKS |
| | LPGSPAIKKGILQSIKIVDELVKVMGGRKPESIVVEMARENQYTNQ |
| | GKSNSQQRLKRLEKSLKELGSKILKENIPAKLSKIDNNALQNDRLY |
| | LYYLQNGKDMYTGDDLDIDRLSNYDIDHIIPQAFLKDNSIDNKVLV |
| | SSASNRGKSDDFPSLEVVKKRKTFWYQLLKSKLISQRKFDNLTKAE |
| | RGGLLPEDKAGFIQRQLVETRQITKHVARLLDEKFNNKKDENNRA |
| | VRTVKIITLKSTLVSQFRKDFELYKVREINDFHHAHDAYLNAVIASA |
| | LLKKYPKLEPEFVYGDYPKYNSFRERKSATEKVYFYSNIMNIFKKSI |
| | SLADGRVIERPLIEVNEETGESVWNKESDLATVRRVLSYPQVNVVK |
| | KVEEQNHGLDRGKPKGLFNANLSSKPKPNSNENLVGAKEYLDPKK |
| | YGGYAGISNSFAVLVKGTIEKGAKKKITNVLEFQGISILDRINYRKD |
| | KLNFLLEKGYKDIELIIELPKYSLFELSDGSRRMLASILSTNNKRGEI |
| | HKGNQIFLSQKFVKLLYHAKRISNTINENHRKYVENHKKEFEELFY |
| | YILEFNENYVGAKKNGKLLNSAFQSWQNHSIDELCSSFIGPTGSERK |
| | GLFELTSRGSAADFEFLGVKIPRYRDYTPSSLLKDATLIHQSVTGLY |
| | ETRIDLAKLGEG |
| SEQ ID NO: 2 | MDKKYSIGLDIGTNSVGWAVITDEYKVPSKKFKVLGNTDRHSIKK |
| | NLIGALLFDSGETAEATRLKRTARRRYTRRKNRICYLQEIFSNEMA |
| | KVDDSFFHRLEESFLVEEDKKHERHPIFGNIVDEVAYHEKYPTIYHL |
| | RKKLVDSTDKADLRLIYLALAHMIKFRGHFLIEGDLNPDNSDVDKL |
| | FIQLVQTYNQLFEENPINASGVDAKAILSARLSKSRRLENLIAQLPG |
| | EKKNGLFGNLIALSLGLTPNFKSNFDLAEDAKLQLSKDTYDDDLDN |
| | LLAQIGDQYADLFLAAKNLSDAILLSDILRVNTEITKAPLSASMIKR |
| | YDEHHQDLTLLKALVRQQLPEKYKEIFFDQSKNGYAGYIDGGASQ |
| | EEFYKFIKPILEKMDGTEELLVKLNREDLLRKQRTFDNGSIPHQIHL |
| | GELHAILRRQEDFYPFLKDNREKIEKILTFRIPYYVGPLARGNSRFA |
| | WMTRKSEETITPWNFEEVVDKGASAQSFIERMTNFDKNLPNEKVL |
| | PKHSLLYEYFTVYNELTKVKYVTEGMRKPAFLSGEQKKAIVDLLF |
| | KTNRKVTVKQLKEDYFKKIECFDSVEISGVEDRFNASLGTYHDLLK |
| | IIKDKDFLDNEENEDILEDIVLTLTLFEDREMIEERLKTYAHLFDDK |
| | VMKQLKRRRYTGWGRLSRKLINGIRDKQSGKTILDFLKSDGFANR |
| | NFMQLIHDDSLTFKEDIQKAQVSGQGDSLHEHIANLAGSPAIKKGIL |
| | QTVKVVDELVKVMGRHKPENIVIEMARENQTTQKGQKNSRER |
| | MKRIEEGIKELGSQILKEHPVENTQLQNEKLYLYYLQNGRDMYVQ |
| | ELDINRLSDYDVDHIVPQSFLKDDSIDNKVLTRSDKNRGKSDNVPS |
| | EEVVKKMKNYWRQLLNAKLITQRKFDNLTKAERGGLSELDKAGFI |
| | KRQLVETRQITKHVAQILDSRMNTKYDENDKLIREVKVITLKSKLV |
| | SDFRKDFQFYKVREINNYHHAHDAYLNAVVGTALIKKYPKLESEF |
| | VYGDYKVYDVRKMIAKSEQEIGKATAKYFFYSNIMNFFKTEITLAN |
| | GEIRKRPLIETNGETGEIVWDKGRDFATVRKVLSMPQVNIVKKTEV |
| | QTGGFSKESILPKRNSDKLIARKKDWDPKKYGGFDSPTVAYSVLVV |
| | AKVEKGKSKKLKSVKELLGITIMERSSFEKNPIDFLEAKGYKEVKK |
| | DLIIKLPKYSLFELENGRKRMLASAGELQKGNELALPSKYVNFLYL |
| | ASHYEKLKGSPEDNEQKQLFVEQHKHYLDEIIEQISEFSKRVILADA |
| | NLDKVLSAYNKHRDKPIREQAENIIHLFTLTNLGAPAAFKYFDTTID |
| | RKRYTSTKEVLDATLIHQSITGLYETRIDLSQLGGD |
| SEQ ID NO: 3 | MKRNYILGLDIGITSVGYGIIDYETRDVIDAGVRLFKEANVENNEGR |
| | RSKRGARRLKRRRRHRIQRVKKLLFDYNLLTDHSELSGINPYEARV |
| | KGLSQKLSEEEFSAALLHLAKRRGVHNVNEVEEDTGNELSTKEQIS |
| | RNSKALEEKYVAELQLERLKKDGEVRGSINRFKTSDYVKEAKQLL |
| | KVQKAYHQLDQSFIDTYIDLLETRRTYYEGPGEGSPFGWKDIKEW |
| | YEMLMGHCTYFPEELRSVKYAYNADLYNALNDLNNLVITRDENE |
| | KLEYYEKFQIIENVFKQKKKPTLKQIAKEILVNEEDIKGYRVTSTGK |
| | PEFTNLKVYHDIKDITARKEIIENAELLDQIAKILTIYQSSEDIQEELT |
| | NLNSELTQEEIEQISNLKGYTGTHNLSLKAINLILDELWHTNDNQIAI |
| | FNRLKLVPKKVDLSQQKEIPTTLVDDFILSPVVKRSFIQSIKVINAIIK |
| | KYGLPNDIIIELAREKNSKDAQKMINEMQKRNRQTNERIEEIIRTTG |
| | KENAKYLIEKIKLHDMQEGKCLYSLEAIPLEDLLNNPFNYEVDHIIP |
| | RSVSFDNSFNNKVLVKQEENSKKGNRTPFQYLSSSDSKISYETFKK |
| | HILNLAKGKGRISKTKKEYLLEERDINRFSVQKDFINRNLVDTRYAT |
| | RGLMNLLRSYFRVNNLDVKVKSINGGFTSFLRRKWKFKKERNKGY |
| | KHHAEDALIIANADFIFKEWKKLDKAKKVMENQMFEEKQAESMPE |
| | IETEQEYKEIFITPHQIKHIKDFKDYKYSHRVDKKPNRELINDTLYST |
| | RKDDKGNTLIVNNLNGLYDKDNDKLKKLINKSPEKLLMYHHDPQT |
| | YQKLKLIMEQYGDEKNPLYKYYEETGNYLTKYSKKDNGPVIKKIK |
| | YYGNKLNAHLDITDDYPNSRNKVVKLSLKPYRFDVYLDNGVYKF |
| | VTVKNLDVIKKENYYEVNSKCYEEAKKLKKISNQAEFIASFYNNDL |
| | IKINGELYRVIGVNNDLLNRIEVNMIDITYREYLENMNDKRPPRIIKT |
| | IASKTQSIKKYSTDILGNLYEVKSKKHPQIIKKG |
| SEQ ID NO: 4 | MSIYQEFVNKYSLSKTLRFELIPQGKTLENIKARGLILDDEKRAKDY |
| | KKAKQIIDKYHQFFIEEILSSVCISEDLLQNYSDVYFKLKKSDDDNL |
| | QKDFKSAKDTIKKQISEYIKDSEKFKNLFNQNLIDAKKGQESDLILW |
| | LKQSKDNGIELFKANSDITDIDEALEIIKSFKGWTTYFKGFHENRKN |
| | VYSSNDIPTSIIYRIVDDNLPKFLENKAKYESLKDKAPEAINYEQIKK |
| | DLAEELTFDIDYKTSEVNQRVFSLDEVFEIANFNNYLNQSGITKENT |
| | IIGGKFVNGENTKRKGINEYINLYSQQINDKTLKKYKMSVLFKQILS |
| | DTESKSFVIDKLEDDSDVVTTMQSFYEQIAAFKTVEEKSIKETLSLL |
| | FDDLKAQKLDLSKIYFKNDKSLTDLSQQVEDDYSVIGTAVLEYITQ |
| | QIAPKNLDNPSKKEQELIAKKTEKAKYLSLETIKLALEEFNKHRDID |
| | KQCRFEEILANFAAIPMIFDEIAQNKDNLAQISIKYQNQGKKDLLQA |
| | SAEDDVKAIKDLLDQTNNLLHKLKIFHISQSEDKANILDKDEHFYL |
| | VFEECYFELANIVPLYNKIRNYITQKPYSDEKFKLNFENSTLANGW |
| | DKNKEPDNTAILFIKDDKYYLGVMNKKNNKIFDDKAIKENKGEGY |
| | KKIVYKLLPGANKMLPKVFFSAKSIKFYNPSEDILRIRNHSTHTKNG |
| | SPQKGYEKFEFNIEDCRKFIDFYKQSISKHPEWKDFGFRFSDTQRYN |
| | SIDEFYREVENQGYKLTFENISESYIDSVVNQGKLYLFQIYNKDFSA |
| | YSKGRPNLHTLYWKALFDERNLQDVVYKLNGEAELFYRKQSIPKK |
| SEQ ID NO: 5 | ITHPAKEAIANKNKDNPKKESVFEYDLIKDKRFTEDKFFFHCPITINF |
| | KSSGANKFNDEINLLLKEKANDVHILSIDRGERHLAYYTLVDGKGN |
| | IIKQDTFNIIGNDRMKTNYHDKLAAIEKDRDSARKDWKKINNIKEM |
| | KEGYLSQVVHEIAKLVIEYNAIVVFEDLNFGFKRGRFKVEKQVYQK |
| | LEKMLIEKLNYLVFKDNEFDKTGGVLRAYQLTAPFETFKKMGKQT |
| | GIIYYVPAGFTSKICPVTGFVNQLYPKYESVSKSQEFFSKFDKICYNL |
| | DKGYFEFSFDYKNFGDKAAKGKWTIASFGSRLINFRNSDKNHNWD |
| | TREVYPTKELEKLLKDYSIEYGHGECIKAAICGESDKKFFAKLTSVL |
| | NTILQMRNSKTGTELDYLISPVADVNGNFFDSRQAPKNMPQDADA |
| | NGAYHIGLKGLMLLGRIKNNQEGKKLNLVIKNEEYFEFVQNRNN |
| | MTQFEGFTNLYQVSKTLRFELIPQGKTLKHIQEQGFIEEDKARNDH |
| | YKELKPIIDRIYKTYADQCLQLVQLDWENLSAAIDSYRKEKTEETR |
| | NALIEEQATYRNAIHDYFIGRTDNLTDAINKRHAEIYKGLFKAELFN |
| | GKVLKQLGTVTTTEHENALLRSFDKFTTYFSGFYENRKNVFSAEDI |
| | STAIPHRIVQDNFPKFKENCHIFTRLITAVPSLREHFENVKKAIGIFVS |
| | TSIEEVFSFPFYNQLLTQTQIDLYNQLLGGISREAGTEKIKGLNEVLN |
| | LAIQKNDETAHIIASLPHRFIPLFKQILSDRNTLSFILEEFKSDEEVIQS |
| | FCKYKTLLRNENVLETAEALFNELNSIDLTHIFISHKKLETISSALCD |
| | HWDTLRNALYERRISELTGKITKSAKEKVQRSLKHEDINLQEIISAA |
| | GKELSEAFKQKTSEILSHAHAALDQPLPTTLKKQEEKEILKSQLDSL |
| | LGLYHLLDWFAVDESNEVDPEFSARLTGIKLEMEPSLSFYNKARNY |
| | ATKKPYSVEKFKLNFQMPTLASGWDVNKEKNNGAILFVKNGLYY |
| | LGIMPKQKGRYKALSFEPTEKTSEGFDKMYYDYFPDAAKMIPKCS |
| | TQLKAVTAHFQTHTTPILLSNNFIEPLEITKEIYDLNNPEKEPKKFQT |
| | AYAKKTGDQKGYREALCKWIDFTRDFLSKYTKTTSIDLSSLRPSSQ |
| | YKDLGEYYAELNPLLYHISFQRIAEKEIMDAVETGKLYLFQIYNKD |
| | FAKGHHGKPNLHTLYWTGLFSPENLAKTSIKLNGQAELFYRPKSR |
| | MKRMAHRLGEKMLNKKLKDQKTPIPDTLYQELYDYVNHRLSHDL |
| | SDEARALLPNVITKEVSHEIIKDRRFTSDKFFFHVPITLNYQAANSPS |
| | KFNQRVNAYLKEHPETPIIGIDRGERNLIYITVIDSTGKILEQRSLNTI |
| | QQFDYQKKLDNREKERVAARQAWSVVGTIKDLKQGYLSQVIHEIV |
| | DLMIHYQAVVVLENLNFGFKSKRTGIAEKAVYQQFEKMLI |
| | DKLNCLVLKDYPAEKVGGVLNPYQLTDQFTSFAKMGTQSGFLFYV |
| | PAPYTSKIDPLTGFVDPFVWKTIKNHESRKHFLEGFDFLHYDVKTG |
| | DFILHFKMNRNLSFQRGLPGFMPAWDIVFEKNETQFDAKGTPFIAG |
| | KRIVPVIENHRFTGRYRDLYPANELIALLEEKGIVFRDGSNILPKLLE |
| | NDDSHAIDTMVALIRSVLQMRNSNAATGEDYINSPVRDLNGVCFD |
| | SRFQNPEWPMDADANGAYHIALKGQLLLNHLKESKDLKLQNGISN |
| | QDWLAYIQELRN |
| SEQ ID NO: 6 | MAVKSIKVKLRLDDMPEIRAGLWKLHKEVNAGVRYYTEWLSLLR |
| | QENLYRRSPNGDGEQECDKTAEECKAELLERLRARQVENGHRGPA |
| | GSDDELLQLARQLYELLVPQAIGAKGDAQQIARKFLSPLADKDAV |
| | GGLGIAKAGNKPRWVRMREAGEPGWEEEKEKAETRKSADRTADV |
| | LRALADFGLKPLMRVYTDSEMSSVEWKPLRKGQAVRTWDRDMF |
| | QQAIERMMSWESWNQRVGQEYAKLVEQKNRFEQKNFVGQEHLV |
| | HLVNQLQQDMKEASPGLESKEQTAHYVTGRALRGSDKVFEKWGK |
| | LAPDAPFDLYDAEIKNVQRRNTRRFGSHDLFAKLAEPEYQALWRE |
| | DASFLTRYAVYNSILRKLNHAKMFATFTLPDATAHPIWTRFDKLGG |
| | NLHQYTFLFNEFGERRHAIRFHKLLKVENGVAREVDDVTVPISMSE |
| | QLDNLLPRDPNEPIALYFRDYGAEQHFTGEFGGAKIQCRRDQLAH |
| | MHRRRGARDVYLNVSVRVQSQSEARGERRPPYAAVFRLVGDNHR |
| | AFVHFDKLSDYLAEHPDDGKLGSEGLLSGLRVMSVDLGLRTSASIS |
| | VFRVARKDELKPNSKGRVPFFFPIKGNDNLVAVHERSQLLKLPGET |
| | ESKDLRAIREERQRTLRQLRTQLAYLRLLVRCGSEDVGRRERSWA |
| | KLIEQPVDAANHMTPDWREAFENELQKLKSLHGICSDKEWMDAV |
| | YESVRRVWRHMGKQVRDWRKDVRSGERPKIRGYAKDVVGGNSIE |
| | QIEYLERQYKFLKSWSFFGKVSGQVIRAEKGSRFAITLREHIDHAKE |
| | DRLKKLADRIIMEALGYVYALDERGKGKWVAKYPPCQLILLEELS |
| | EYQFNNDRPPSENNQLMQWSHRGVFQELINQAQVHDLLVGTMYA |
| | AFSSRFDARTGAPGIRCRRVPARCTQEHNPEPFPWWLNKFVVEHTL |
| | DACPLRADDLIPTGEGEIFVSPFSAEEGDFHQIHADLNAAQNLQQRL |
| | WSDFDISQIRLRCDWGEVDGELVLIPRLTGKRTADSYSNKVFYTNT |
| | GVTYYERERGKKRRKVFAQEKLSEEEAELLVEADEAREKSVVLMR |
| | DPSGIINRGNWTRQKEFWSMVNQRIEGYLVKQIRSRVPLQDSACEN |
| | TGDI |
| SEQ ID NO: 7 | YGRKKRRQRRR |
| SEQ ID NO: 8 | RRQRRTSKLMKR |
| SEQ ID NO: 9 | GWTLNSAGYLLGKINLKALAALAKKIL |
| SEQ ID NO: 10 | KALAWEAKLAKALAKALAKHLAKALAKALKCEA |
| SEQ ID NO: 11 | RQIKIWFQNRRMKWKK |
| SEQ ID NO: 12 | YGRKKRRQRRR |
| SEQ ID NO: 13 | RKKRRQRRR |
| SEQ ID NO: 14 | YGRKKRRQRRR |
| SEQ ID NO: 15 | RKKRRQRR |
| SEQ ID NO: 16 | YARAAARQARA |
| SEQ ID NO: 17 | THRLPRRRRRR |
| SEQ ID NO: 18 | GGRRARRRRRR |

### Nucleotides

i. Sequence of Interest

In some embodiments, a polynucleotide of the disclosure is an exogenous polynucleotide which comprises a sequence of interest (SOI) to be inserted into the genome of a eukaryotic cell. In some embodiments, the sequence of interest encodes a gene of interest.

In some embodiments, the polynucleotide comprising exogenous polynucleotide comprising a SOI is an exogenous polynucleotide template which is inserted into the genome of a eukaryotic cell via CRISPR/Cas-mediated homologous recombination. In some embodiments, the SOI comprises at least one mutation of interest to be inserted into a genome of a eukaryotic cell. In some embodiments, the SOI comprises a gene of interest to be inserted into a genome of a eukaryotic cell. In some embodiments, the SOI can be introduced as an exogenous polynucleotide template. In some embodiments, the SOI is a hybrid polynucleotide comprising single-stranded and double-stranded regions. In some embodiments, the hybrid polynucleotide comprises double-stranded sequences at the 5′ and 3′ ends and an internal single-stranded sequence (Shy et al, bioRxiv, 2021, preprint published Sep. 2, 2021). In some embodiments, the exogenous polynucleotide includes blunt ends. In some embodiments, the exogenous polynucleotide template includes cohesive ends. In some embodiments, the exogenous polynucleotide template includes cohesive ends complementary to cohesive ends in the target sequence.

The exogenous polynucleotide template can be of any suitable length, such as about or at least about 10, 15, 20, 25, 50, 75, 100, 150, 200, 250, 500, 1000, 5000, or 10,000 or more nucleotides in length. In some embodiments, the exogenous polynucleotide template is complementary to a portion of a polynucleotide including the target sequence. In some embodiments, when optimally aligned, the exogenous polynucleotide template overlaps with one or more nucleotides of a target sequence (e.g., about or at least about 1, 5, 10, 15, 20, 25, 30, 35, 40, 45, 50, 60, 70, 80, 90, or 100 or more nucleotides). In some embodiments, when the exogenous polynucleotide template and a polynucleotide including the target sequence are optimally aligned, the nearest nucleotide of the exogenous polynucleotide template is within about 1, 5, 10, 15, 20, 25, 50, 75, 100, 200, 300, 400, 500, 100, 1500, 2000, 2500, 5000, 10,000 or more nucleotides from the target sequence.

In some embodiments, the exogenous polynucleotide is DNA, such as, e.g., a DNA plasmid, a bacterial artificial chromosome (BAC), a yeast artificial chromosome (YAC), a viral vector, a linear piece of single-stranded or double-stranded DNA, an oligonucleotide, a PCR fragment, a naked nucleic acid, or a nucleic acid complexed with a delivery vehicle such as a liposome. In some embodiments, the exogenous polynucleotide is RNA. In some embodiments, the RNA is a messenger RNA (mRNA).

In some embodiments, the exogenous polynucleotide is inserted into the target sequence using an endogenous DNA repair pathway of the cell. In some embodiments, the endogenous DNA repair pathway is HDR. During the repair process, an exogenous polynucleotide template including the SOI can be introduced into the target sequence. In some embodiments, an exogenous polynucleotide template including the SOI flanked by an upstream sequence and a downstream sequence is introduced into the cell, where the upstream and downstream sequences share sequence similarity with either side of the site of integration in the target sequence. In some embodiments, the exogenous polynucleotide including the SOI includes, for example, a mutated gene. In some embodiments, the exogenous polynucleotide includes a sequence endogenous or exogenous to the cell. In some embodiments, the SOI includes polynucleotides encoding a protein, or a non-coding sequence such as, e.g., a microRNA. In some embodiments, the SOI is operably linked to a regulatory element. In some embodiments, the SOI is a regulatory element. In some embodiments, the SOI includes a resistance cassette, e.g., a gene that confers resistance to an antibiotic. In some embodiments, the SOI includes a mutation of the wild-type target sequence. In some embodiments, the SOI disrupts or corrects the target sequence by creating a frameshift mutation or nucleotide substitution. In some embodiments, the SOI includes a marker. Introduction of a marker into a target sequence can make it easy to screen for targeted integrations. In some embodiments, the marker is a restriction site, a fluorescent protein, or a selectable marker. In some embodiments, the SOI is introduced as a vector including the SOI.

The upstream and downstream sequences in the exogenous polynucleotide template are selected to promote homologous recombination between the target sequence and the exogenous polynucleotide. The upstream sequence is a nucleic acid sequence that shares sequence similarity with the sequence upstream of the targeted site for integration (i.e., the target sequence). Similarly, the downstream sequence is a nucleic acid sequence that shares sequence similarity with the sequence downstream of the targeted site for integration. Thus, in some embodiments, the exogenous polynucleotide template including the SOI is inserted into the target sequence by homologous recombination at the upstream and downstream sequences. In some embodiments, the upstream and downstream sequences in the exogenous polynucleotide template have at least 70%, at least 75%, at least 80%, at least 85%, at least 90%, at least 95%, at least 96%, at least 97%, at least 98%, at least 99%, or 100% sequence identity with the upstream and downstream sequences of the targeted genome sequence, respectively. In some embodiments, the upstream or downstream sequence has at least about 20, 50, 100, 150, 200, 250, 300, 350, 400, or 500 base pairs and up to about 600, 750, 1000, 1250, 1500, 1750 or 2000 base pairs. In some embodiments, the upstream or downstream sequence has about 20 to 2000 base pairs, or about 50 to 1750 base pairs, or about 100 to 1500 base pairs, or about 200 to 1250 base pairs, or about 300 to 1000 base pairs, or about 400 to about 750 base pairs, or about 500 to 600 base pairs. In some embodiments, the upstream or downstream sequence has about 50, about 100, about 250, about 500, about 100, about 1250, about 1500, about 1750, about 2000, about 2250, or about 2500 base pairs.

In some embodiments, the SOI comprises a gene of interest. As used herein, the term “gene of interest” refers to a gene that encodes a biomolecule of interest (e.g., a protein or an RNA molecule). In some embodiments, the gene of interest encodes a protein of interest. In some embodiments, the protein of interest comprises an intracellular protein, a membrane protein, an extracellular protein, or combination thereof. In some embodiments, the protein of interest comprises a nuclear protein, a transcription factor, a nuclear membrane transporter, an intracellular organelle associated protein, a membrane receptor, a catalytic protein, an enzyme, a therapeutic protein, a membrane protein, a membrane transport protein, a signal transduction protein, an immunological protein, or combination thereof. In some embodiments, the immunological protein comprises an antibody, e.g., IgG, IgA, IgM, IgD, IgE, or combination thereof. In some embodiments, the immunological protein is a T cell receptor (TCR). In some embodiments, immunological protein is a chimeric antigen receptor (CAR). In some embodiments, the SOI encodes a copy of a native gene of the host cell. In some embodiments, the SOI encodes a copy of a native gene that is deficient in the host cell. In some embodiments, the host cell comprises a mutation in a gene, and the SOI encodes a wild-type copy of the gene. In some embodiments, the host cell comprises a wild-type gene, and the SOI encodes a copy of the gene comprising a mutation of interest. In some embodiments, the SOI encodes a heterologous gene that is not naturally occurring in the host cell.

In some embodiments, the gene of interest encodes an RNA of interest. In some embodiments, the RNA of interest comprises a therapeutic RNA. In some embodiments, the RNA of interest comprises messenger RNA (mRNA), transfer RNA (tRNA), ribosomal RNA (rRNA), small nuclear RNA (snRNA), antisense RNA, microRNA (miRNA), small interfering RNA (siRNA), cell-free RNA (cfRNA), or combination thereof. In some embodiments, the sequence of interest comprises a regulatory element of interest. In some embodiments, the SOI is inserted into a target polynucleotide of a host cell, such that the regulatory element on the sequence of interest is capable of regulating a native gene of the host cell. Regulatory elements are described herein and include, e.g., promoters, enhancers, silencers, operators, response elements, 5′ UTR, 3′ UTR, insulators, and the like.

In some embodiments, the polynucleotide comprising a SOI is about 1 nucleotide to about 5000 nucleotides in length. In some embodiments, the polynucleotide comprising the SOI is about 5 nucleotides to about 5000 nucleotides in length. In some embodiments, polynucleotide comprising a SOI is about 6 nucleotides to about 1000 nucleotides in length. In some embodiments, the polynucleotide comprising a SOI is about 7 nucleotides to about 750 nucleotides in length. In some embodiments, the polynucleotide comprising a SOI is about 8 nucleotides to about 500 nucleotides in length. In some embodiments, the polynucleotide comprising a SOI is about 9 nucleotides to about 250 nucleotides in length. In some embodiments, the polynucleotide comprising a SOI is about 10 nucleotides to about 100 nucleotides in length. In some embodiments, the polynucleotide comprising a SOI is about 15 nucleotides to about 90 nucleotides in length. In some embodiments, the polynucleotide comprising a SOI is about 20 nucleotides to about 80 nucleotides in length. In some embodiments, the polynucleotide comprising a SOI is about 25 nucleotides to about 70 nucleotides in length. In some embodiments, the polynucleotide comprising a SOI is about 30 nucleotides to about 50 nucleotides in length. In some embodiments, the polynucleotide comprising a SOI is about 1 to about 10 nucleotides in length. In some embodiments, the polynucleotide comprising a SOI is about 1 to about 20 nucleotides in length. In some embodiments, the polynucleotide comprising a SOI is about 1 to about 30 nucleotides in length. In some embodiments, the polynucleotide comprising a SOI is about 10 to about 40 nucleotides in length. In some embodiments, the polynucleotide comprising a SOI is about 1 to about 50 nucleotides in length. In some embodiments, the polynucleotide comprising a SOI is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the polynucleotide comprising a SOI is greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

In some embodiments, the SOI is about 3 to about 5000 nucleotides in length. In some embodiments, the SOI is about 4 to about 1000 nucleotides in length. In some embodiments, the SOI is about 5 to about 900 nucleotides in length. In some embodiments, the SOI is about 6 to about 800 nucleotides in length. In some embodiments, the SOI is about 7 to about 700 nucleotides in length. In some embodiments, the SOI is about 8 to about 600 nucleotides in length. In some embodiments, the SOI is about 9 to about 500 nucleotides in length. In some embodiments, the SOI is about 50 to about 5000 nucleotides in length. In some embodiments, the SOI is about 60 to about 1000 nucleotides in length. In some embodiments, the SOI is about 70 to about 900 nucleotides in length. In some embodiments, the SOI is about 8 to about 800 nucleotides in length. In some embodiments, the SOI is about 90 to about 700 nucleotides in length. In some embodiments, the SOI is about 100 to about 500 nucleotides in length. In some embodiments, the SOI is about 100 to about 250 nucleotides in length. In some embodiments, the SOI is about 10 to about 90 nucleotides in length. In some embodiments, the SOI is about 11 to about 80 nucleotides in length. In some embodiments, the SOI is about 12 to about 70 nucleotides in length. In some embodiments, the SOI is about 15 to about 60 nucleotides in length. In some embodiments, the SOI is about 10 to about 50 nucleotides in length. In some embodiments, the SOI is about 1 to about 10 nucleotides in length. In some embodiments, the SOI is about 1 to about 25 nucleotides in length. In some embodiments, the SOI is about 1 to about 50 nucleotides in length. In some embodiments, the SOI is about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides in length. In some embodiments, the SOI is greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

ii. Cas and Cas-Associated Polynucleotides

In some embodiments, the present disclosure encompasses nucleotide or polynucleotide sequences which encode a Cas effector protein of the disclosure, i.e., a Cas polynucleotide.

In some embodiments, a polynucleotide of the disclosure is capable of forming a complex with a Cas effector protein. In some embodiments, the polynucleotide capable of forming a complex with a Cas effector protein comprise a guide sequence. In some embodiments, the polynucleotide capable of forming a complex with a Cas effector protein comprises a Cas-binding region. In some embodiments, the polynucleotide capable of forming a complex with a Cas effector protein comprises a DNA template sequence. In some embodiments, the polynucleotide capable of forming a complex with a Cas effector protein comprises a guide sequence, a Cas-binding region, and a DNA template sequence, or any combination thereof. In some embodiments, the polynucleotide comprises, in 5′ to 3′ order, a guide sequence, a Cas-binding region, and a DNA template sequence.

In some embodiments, the guide sequence is capable of hybridizing with a target polynucleotide, e.g., a target polynucleotide in a genome of a host cell. In embodiments, the guide sequence is complementary to the target polynucleotide. In some embodiments, the target polynucleotide is a target DNA intended to be cleaved by the Cas nuclease or Cas nickase. In some embodiments, the guide sequence comprises RNA, i.e., an RNA guide sequence. In some embodiments, the guide sequence comprises a combination of RNA and DNA. Hybrid RNA-DNA guide sequences are further described in, e.g., Rueda et al., Nat Comm 8:1610 (2017).

In some embodiments, the guide sequence is about 10 to about 40 nucleotides in length. In some embodiments, the guide sequence is about 12 to about 30 nucleotides in length. In some embodiments, the guide sequence is about 15 to about 20 nucleotides in length. In some embodiments, the guide sequence is about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 nucleotides in length. In some embodiments, the guide sequence is a sufficient length for hybridizing to the target polynucleotide.

In some embodiments, the Cas-binding region is capable of binding to the Cas effector protein (e.g., Cas nuclease or Cas nickase), thereby forming a complex with the Cas protein. In some embodiments, the Cas-binding region comprises RNA. In some embodiments, the Cas-binding region comprises a combination of RNA and DNA. Hybrid RNA-DNA sequences that can bind to and/or activate Cas proteins are further described in, e.g., Rueda et al., Nat Comm 8:1610 (2017).

In some embodiments, multiple guide RNA as described in the methods, kits, and compositions described herein can be used during the same method, kit or composition. For example, in some embodiments, 2, 3, 4, 5, 6, 7, 8, 9 or 10 or more different guide RNA can be used at the same time.

In some embodiments, the Cas-binding region comprises a tracrRNA that binds to and activates the Cas protein. In some embodiments, the Cas-binding region is capable of hybridizing with a tracrRNA, and the composition further comprises a tracrRNA. In some embodiments, the tracrRNA is capable of binding the Cas nuclease or Cas nickase. In some embodiments, the tracrRNA is capable of activating the Cas nuclease or Cas nickase. In some embodiments, the activating comprises initiating or increasing the cleavage activity of the Cas nuclease or Cas nickase. In some embodiments, the activating comprises promoting binding of the Cas nuclease or Cas nickase to a target polynucleotide (e.g., as guided by the guide sequence). In some embodiments, the activating comprises a combination of promoting binding of the Cas nuclease or Cas nickase to the target polynucleotide; and initiating or increasing cleavage activity of the Cas nuclease or Cas nickase. TracrRNA sequences of Cas proteins (e.g., Cas9, Cas12a, or Type II-B Cas proteins described herein) are available from public databases, including RNA central and Rfam, and further described in, e.g., Chylinski et al., RNA Biol 10 (5): 726-737 (2013) and Gasiunas et al., Nat Comm 11:5512 (2020).

In some embodiments, the polynucleotide capable of forming a complex with a Cas effector molecule comprises a DNA template sequence at a 3′ end of the polynucleotide. In some embodiments, the DNA template sequence comprises single-stranded DNA. In some embodiments, the DNA template sequence comprises a sequence of interest. In some embodiments, the DNA template sequence comprises a primer binding sequence and a sequence of interest. In some embodiments, the DNA template sequence comprises a template for amplification by a DNA polymerase. In some embodiments, the sequence of interest comprises a template for amplification by a DNA polymerase. In some embodiments, the Cas nuclease or Cas nickase of the composition is guided to a target polynucleotide by the guide sequence and cleaves the target polynucleotide, and one strand of the cleaved target polynucleotide hybridizes to the primer binding sequence and serves as a primer for a DNA polymerase. In some embodiments, the DNA polymerase is capable of synthesizing a DNA strand complementary to the SOI to form a double-stranded sequence comprising the SOI. In some embodiments, the double-stranded sequence comprising the SOI is inserted into the cleaved target polynucleotide, e.g., via ligation or a DNA repair pathway described herein.

In some embodiments, the DNA template sequence is about 5 nucleotides to about 5000 nucleotides in length. In some embodiments, the DNA template sequence is about 6 nucleotides to about 1000 nucleotides in length. In some embodiments, the DNA template sequence is about 7 nucleotides to about 750 nucleotides in length. In some embodiments, the DNA template sequence is about 8 nucleotides to about 500 nucleotides in length. In some embodiments, the DNA template sequence is about 9 nucleotides to about 250 nucleotides in length. In some embodiments, the DNA template sequence is about 10 nucleotides to about 100 nucleotides in length. In some embodiments, the DNA template sequence is about 15 nucleotides to about 90 nucleotides in length. In some embodiments, the DNA template sequence is about 20 nucleotides to about 80 nucleotides in length. In some embodiments, the DNA template sequence is about 25 nucleotides to about 70 nucleotides in length. In some embodiments, the DNA template sequence is about 30 nucleotides to about 50 nucleotides in length. In some embodiments, the DNA template sequence is about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the DNA template sequence is greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

In some embodiments, the DNA template sequence comprises a primer-binding sequence. In some embodiments, the primer-binding sequence is about 3 to about 50 nucleotides in length. In some embodiments, the primer-binding sequence is about 4 to about 45 nucleotides in length. In some embodiments, the primer-binding sequence is about 5 to about 40 nucleotides in length. In some embodiments, the primer-binding sequence is about 6 to about 35 nucleotides in length. In some embodiments, the primer-binding sequence is about 7 to about 30 nucleotides in length. In some embodiments, the primer-binding sequence is about 8 to about 25 nucleotides in length. In some embodiments, the primer-binding sequence is about 10 to about 20 nucleotides in length. In some embodiments, the primer-binding sequence is about 4 to about 30 nucleotides in length. In some embodiments, the primer-binding sequence is about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the primer-binding sequence is of sufficient length to hybridize with a region of the cleaved target DNA sequence.

In some embodiments, the polynucleotide comprising the DNA template sequence comprises a modified nucleotide, a non-B DNA structure, a DNA polymerase recruitment moiety, a DNA ligase recruitment moiety, or a combination thereof.

In some embodiments, the polynucleotide comprising DNA template sequence comprises a modified nucleotide. In some embodiments, the modified nucleotide comprises an abasic site, a covalent linker, a xeno nucleic acid (XNA), a locked nucleic acid (LNA), a peptide nucleic acid (PNA), a phosphorothioate bond, a DNA lesion, a DNA photoproduct, a modified deoxyribonucleoside, a methylated nucleotide, or a combination thereof.

In some embodiments, the modified nucleotide reduces or prevents overextension of the sequence of interest by the DNA polymerase. In some embodiments, reducing or preventing overextension of the sequence of interest by the DNA polymerase increases the precision of inserting the double-stranded sequence comprising the sequence of interest. In some embodiments, the modified nucleotide comprises an abasic site, also known as an apurinic/apyrimidinic (AP site). In some embodiments, the modified nucleotide comprises a covalent linker. In some embodiments, the covalent linker comprises a triethylene glycol (TEG) linker. In some embodiments, the covalent linker comprises an amino linker. TEG linkers and amino linkers have been shown to block polymerase extension; see, e.g., Strobel et al., bioRxiv doi: 10.1101/2019.12.26.888743 (23 Jan. 2020).

In some embodiments, the modified nucleotide reduces or prevents nuclease degradation of a polynucleotide of the disclosure. In some embodiments, the modified nucleotide comprises a xeno nucleic acid (XNA). An XNA is a synthetic nucleotide analogue that has a different sugar group than the deoxyribose of DNA or the ribose of RNA. Exemplary sugar groups for XNA include, but are not limited to, threose, cyclohexene, glycol, or a locked ribose. In some embodiments, the XNA comprises 1,5-anhydrohexitol nucleic acid (HNA), cyclohexene nucleic acid (CeNA), threose nucleic acid (TNA), glycol nucleic acid (GNA), locked nucleic acid (LNA), and peptide nucleic acid (PNA). In some embodiments, the modified nucleotide comprises a locked nucleic acid (LNA), also known as a bridged nucleic acid (BNA). An LNA is a modified RNA nucleotide in which the ribose moiety is modified with an extra bridge connecting the 2′ oxygen and 4′ carbon. In some embodiments, the modified nucleotide comprises a peptide nucleic acid (PNA). Unlike the deoxyribose or ribose backbones of DNA or RNA, the backbone of a PNA polymer comprises N-(2-aminoethyl)-glycine units linked by peptide bonds, and the purine and pyrimidine bases are linked to the PNA backbone by a methylene bridge and a carbonyl group. In some embodiments, the modified nucleotide comprises a phosphorothioate bond. A phosphorothioate bond comprises a sulfur atom in place of one of the oxygens in the phosphate group linking two nucleotides. In some embodiments, the presence of an XNA, e.g., an LNA or a PNA, or a phosphorothioate bond in a polynucleotide increases stability of the polynucleotide against nuclease degradation.

In some embodiments, the presence of a modified nucleotide in a polynucleotide (e.g., the polynucleotide of the composition provided herein) is capable of recruiting a DNA polymerase to the polynucleotide. In some embodiments, recruiting a DNA polymerase comprises: increasing the likelihood that a DNA polymerase recognizes the polynucleotide, e.g., due to presence of the modified nucleotide therein; promoting binding of a DNA polymerase to the polynucleotide; and/or activating a DNA polymerase, e.g., initiating or increasing activity of the DNA polymerase. In some embodiments, the recruited DNA polymerase binds to a strand of the cleaved target polynucleotide and extends the sequence of interest on the DNA template sequence, as described herein.

In some embodiments, the modified nucleotide comprises a DNA lesion. As used herein, a “DNA lesion” refers to a region of a DNA polynucleotide containing a base alteration, base deletion, and/or sugar alteration typically indicative of DNA damage. DNA lesions can be caused by hydrolysis, oxidation, alkylation, depurination, depyrimidination, and/or deamination of a nucleobase. In some embodiments, the DNA lesion is capable of recruiting a DNA polymerase. In some embodiments, the DNA lesion comprises 8-oxoguanine, thymine-glycol, N7-(2-hydroxethyl) guanine (7HEG), 7-(2-oxoethyl) guanine, or a combination thereof. In some embodiments, the DNA lesion comprises 8-oxoguanine, thymine-glycol, or a combination thereof.

In some embodiments, the modified nucleotide comprises a DNA photoproduct. DNA photoproducts are ultraviolet (UV)-induced DNA lesions and are further described in, e.g., Yokoyama et al., Int J Mol Sci 15 (11): 20321-20338 (2014). In some embodiments, the DNA photoproduct is capable of recruiting a DNA polymerase. In some embodiments, the DNA photoproduct comprises a pyrimidine dimer, a cyclobutane pyrimidine dimer (CPD), a pyrimidine (6-4) pyrimidone photoproduct (also referred to as a “(6-4) photoproduct”), an adenine-thymine heterodimer, a Dewar pyrimidinone, or a combination thereof. In some embodiments, the DNA photoproduct comprises CPD, a (6-4) photoproduct, or a combination thereof.

In some embodiments, the modified nucleotide comprises a modified deoxyribonucleoside. In some embodiments, the modified deoxyribonucleoside is capable of recruiting a DNA polymerase. In some embodiments, the modified deoxyribonucleoside comprises a base not typically present in DNA, i.e., adenine, cytosine, guanine, or thymine. In some embodiments, the modified deoxyribonucleoside comprises deoxyuridine, acrolein-deoxyguanine, malondialdehyde-deoxyguanine, deoxyinosine, deoxyxanthosine, or a combination thereof. In some embodiments, the modified deoxyribonucleoside comprises deoxyuridine.

In some embodiments, the modified nucleotide comprises one or more methylated nucleotides. In some embodiments, methylated nucleotides, e.g., methylated cytosines, are capable of recruiting a DNA polymerase. In some embodiments, the methylated nucleotide comprises 5-hydroxymethylcytosine, 5-methylcytosine, or a combination thereof.

In some embodiments, the DNA template sequence comprises a non-B DNA structure. As used herein, “a non-B DNA structure” is a DNA secondary structural conformation that is not the canonical right-handed B-DNA helix. Non-limiting examples of non-B DNA structures include G-quadruplex, triplex DNA (H-DNA), Z-DNA, cruciform, slipped DNA strands, A-tract bending, sticky DNA. Non-B DNA structures are further described in, e.g., Guiblet et al., Nucleic Acids Res 49 (3): 1497-1516 (2021). In some embodiments, the non-B DNA structure is capable of recruiting a DNA polymerase. In some embodiments, the non-B DNA structure comprises a hairpin, a cruciform, Z-DNA, H-DNA (triplex DNA), G-quadruplex DNA (tetraplex DNA), slipped DNA, sticky DNA, or a combination thereof.

In some embodiments, the DNA template sequence comprises a DNA polymerase recruitment moiety. DNA polymerase recruitment is described herein. Non-limiting examples of DNA polymerases that can be recruited by the DNA polymerase recruitment moiety include bacterial DNA polymerases such as Pol I (including a Klenow fragment thereof), Pol II, Pol III, Pol IV, or Pol V; eukaryotic DNA polymerases such as Pol α, Pol β, Pol λ, Pol γ, Pol σ, Pol μ, Pol δ, Pol ε, Pol η, Pol ι, Pol κ, Pol ζ, Pol θ, REV1, or REV3; isothermal DNA polymerases such as Bst, T4, or Φ29 (phi29) DNA polymerase; thermostable DNA polymerases such as Taq, Pfu, KOD, Tth, or Pwo DNA polymerase; or a variant or homologue thereof.

In some embodiments, a polynucleotide of the disclosure can be chemically crosslinked to one or more moieties or conjugates which enhance the activity, cellular distribution, or cellular uptake of the polynucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus of an exogenous polypeptide (e.g., a site-directed modifying polypeptide). In some embodiments, a PTD is covalently linked to the carboxyl terminus of an exogenous polypeptide (e.g., a site-directed modifying polypeptide). In some embodiments, a PTD is covalently linked to a nucleic acid (e.g., a DNA-targeting RNA, a polynucleotide encoding a DNA-targeting RNA, a polynucleotide encoding a site-directed modifying polypeptide, etc.). Exemplary PTDs include but are not limited to a minimal undecapeptide protein transduction domain (corresponding to residues 47-57 of HIV-1 TAT comprising YGRKKRRQRRR; SEQ ID NO:7); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9 (6): 489-96); an Drosophila Antennapedia protein transduction domain (Noguchi et al. (2003) Diabetes 52 (7): 1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:8); Transportan GWTLNSAGYLLGKINLKALAALAKKIL (SEQ ID NO: 9); KALAWEAKLAKALAKALAKHLAKALAKALKCEA (SEQ ID NO:10); and RQIKIWFQNRRMKWKK (SEQ ID NO:11). Exemplary PTDs include but are not limited to, YGRKKRRQRRR (SEQ ID NO:12), RKKRRQRRR (SEQ ID NO: 13); an arginine homopolymer of from 3 arginine residues to 50 arginine residues; Exemplary PTD domain amino acid sequences include, but are not limited to, any of the following: YGRKKRRQRRR (SEQ ID NO:14); RKKRRQRR (SEQ ID NO:15); YARAAARQARA (SEQ ID NO:16); THRLPRRRRRR (SEQ ID NO: 17); and GGRRARRRRRR (SEQ ID NO:18). In some embodiments, the PTD is an activatable CPP (ACPP) (Aguilera et al. (2009) Integr Biol (Camb) June; 1 (5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some embodiments, a polynucleotide of the disclosure is codon optimized for expression in a eukaryotic cell. In some embodiments, the polynucleotide sequence encoding a stiCas9 is codon optimized for expression in an animal cell. In some embodiments, the polynucleotide sequence encoding the recombinant Cas effector protein is codon optimized for expression in a human cell. In some embodiments, the polynucleotide sequence encoding the recombinant Cas effector protein is codon optimized for expression in a plant cell. Codon optimization is the adjustment of codons to match the expression host's tRNA abundance in order to increase yield and efficiency of recombinant or heterologous protein expression. Codon optimization methods are routine in the art and may be performed using software programs such as, for example, Integrated DNA Technologies' Codon Optimization tool, Entelechon's Codon Usage Table analysis tool, GENEMAKER's Blue Heron software, Aptagen's Gene Forge software, DNA Builder Software, General Codon Usage Analysis software, the publicly available OPTIMIZER software, and Genscript's OptimumGene algorithm.

### CRISPR-Cas Systems

In some embodiments, the present disclosure encompasses CRISPR-Cas systems comprising a naturally-occurring Cas effector protein or a non-naturally occurring Cas effector protein, and a polynucleotide encoding a sequence of interest. In some embodiments, the CRISPR-Cas system comprises a naturally-occurring Cas effector protein or non-naturally occurring Cas effector protein, a polynucleotide encoding a sequence of interest, and a polynucleotide capable of forming a complex with a Cas effector protein. In some embodiments, the polynucleotide capable of forming a complex with a Cas effector protein comprises a guide sequence, a Cas-binding region, and a DNA template region.

In some embodiments, the CRISPR-Cas system comprises a regulatory element operably linked to a polynucleotide sequence encoding a recombinant Cas effector protein provided herein, and polynucleotide that forms a complex with the recombinant Cas effector protein and includes a guide sequence.

In some embodiments, the regulatory element linked to the polynucleotide sequence encoding a recombinant Cas effector protein is a promoter. In some embodiments, the regulatory element is a eukaryote promoter. In some embodiments, the regulatory element is a viral promoter. In some embodiments, the regulatory element is a eukaryotic regulatory element, i.e., a eukaryotic promoter. In some embodiments, the eukaryotic regulatory element is a mammalian promoter.

In some embodiments, the polynucleotide capable of forming a complex with the Cas effector protein of the CRISPR-Cas system is an RNA molecule. An RNA molecule that binds to CRISPR-Cas components and targets them to a specific location within the target DNA is referred to herein as “guide RNA,” “gRNA,” or “small guide RNA” and may also be referred to herein as a “DNA-targeting RNA.” A guide polynucleotide, e.g., guide RNA, includes at least two nucleotide segments: at least one “DNA-binding segment” and at least one “polypeptide-binding segment.” By “segment” is meant a part, section, or region of a molecule, e.g., a contiguous stretch of nucleotides of guide polynucleotide molecule. The definition of “segment,” unless otherwise specifically defined, is not limited to a specific number of total base pairs.

In some embodiments, the DNA-binding segment (or “DNA-targeting sequence”) of the guide polynucleotide hybridizes with a target sequence in a cell. In some embodiments, the DNA-binding segment of the guide polynucleotide, e.g., guide RNA, includes a polynucleotide sequence that is complementary to a specific sequence within a target DNA.

In some embodiments, the guide polynucleotide of the present disclosure has a guide sequence that hybridizes to a target sequence in a eukaryotic cell. In some embodiments, the eukaryotic cell is an animal or human cell. In some embodiments, the eukaryotic cell is a human or rodent or bovine cell line or cell strain. Examples of such cells, cell lines, or cell strains include, but are not limited to, mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EB1, EB2, EB3, oncolytic or hybridoma-cell lines. In some embodiments, the eukaryotic cells are CHO-cell lines. In some embodiments, the eukaryotic cell is a CHO cell. In some embodiments, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the POTELLIGENT CHOK1 SV (Lonza Biologics, Inc.). Eukaryotic cells can also be avian cells, cell lines or cell strains, such as, for example, EBX cells, EB14, EB24, EB26, EB66, or EBv13.

In some embodiments, the eukaryotic cell is a human cell. In some embodiments, the human cell is a stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In some embodiments, the human cell is a differentiated form of any of the cells described herein. In some embodiments, the eukaryotic cell is a cell derived from any primary cell in culture.

In some embodiments, the eukaryotic cell is a hepatocyte such as a human hepatocyte, animal hepatocyte, or a non-parenchymal cell. For example, the eukaryotic cell can be a plateable metabolism qualified human hepatocyte, a plateable induction qualified human hepatocyte, plateable human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In some embodiments, the eukaryotic cell is a plant cell. For example, the plant cell can be of a crop plant such as cassava, corn, sorghum, wheat, or rice. The plant cell can be of an algae, tree, or vegetable. The plant cell can be of a monocot or dicot or of a crop or grain plant, a production plant, fruit, or vegetable. For example, the plant cell can be of a tree, e.g., a citrus tree such as orange, grapefruit, or lemon tree; peach or nectarine trees; apple or pear trees; nut trees such as almond or walnut or pistachio trees; nightshade plants, e.g., potatoes, plants of the genus Brassica, plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

In some embodiments, the guide sequence of the guide polynucleotide is about 5 to about 50 nucleotides. In some embodiments, the guide sequence of the guide polynucleotide is about 6 to about 45 nucleotides. In some embodiments, the guide sequence of the guide polynucleotide is about 7 to about 40 nucleotides. In some embodiments, the guide sequence of the guide polynucleotide is about 8 to about 35 nucleotides. In some embodiments, the guide sequence of the guide polynucleotide is about 9 to about 30 nucleotides. In some embodiments, the guide sequence of the guide polynucleotide is about 10 to about 20 nucleotides. In some embodiments, the guide sequence of the guide polynucleotide is about 12 to about 20 nucleotides. In some embodiments, the guide sequence of the guide polynucleotide is about 14 to about 20 nucleotides. In some embodiments, the guide sequence of the guide polynucleotide is about 16 to about 20 nucleotides. In some embodiments, the guide sequence of the guide polynucleotide is about 18 to about 20 nucleotides. In some embodiments, the guide sequence of the guide polynucleotide is about 5 to about 10 nucleotides. In some embodiments, the guide sequence of the guide polynucleotide is about 6 to about 10 nucleotides. In some embodiments, the guide sequence of the guide polynucleotide is about 7 to about 10 nucleotides. In some embodiments, the guide sequence of the guide polynucleotide is about 8 to about 10 nucleotides. The length of the guide sequence may be determined by the skilled artisan using guide sequence design tools such as, e.g., CRISPR Design Tool (Hsu et al., Nat Biotechnol 31 (9): 827-832 (2013)), ampliCan (Labun et al., bioRxiv 2018, doi: 10.1101/249474), CasFinder (Alach et al., bioRxiv 2014, doi: 10.1101/005074), CHOPCHOP (Labun et al., Nucleic Acids Res 2016, doi: 10.1093/nar/gkw398), and the like.

In some embodiments, the guide polynucleotide, e.g., guide RNA, of the present disclosure includes a polypeptide-binding sequence/segment. The polypeptide-binding segment (or “protein-binding sequence”) of the guide polynucleotide, e.g., guide RNA, interacts with the polynucleotide-binding domain of a Cas effector protein of the present disclosure. Such polypeptide-binding segments or sequences are known to those of skill in the art, e.g., those disclosed in U.S. Patent Publications 2014/0068797, 2014/0273037, 2014/0273226, 2014/0295556, 2014/0295557, 2014/0349405, 2015/0045546, 2015/0071898, 2015/0071899, and 2015/0071906, the disclosures of which are incorporated herein in their entireties. In some embodiments, the polypeptide-binding segment of the guide polynucleotide binds to Cas9. In some embodiments, the polypeptide-binding segment of the guide polynucleotide binds to the recombinant Cas9 proteins provided herein.

In some embodiments, the guide polynucleotide is at least about 10, 15, 20, 25 or 30 nucleotides and up to about 25, 30, 35, 40, 45, 50, 60, 70, 80, 90, 100, 110, 120, 130, 140 or 150 nucleotides. In some embodiments, the guide polynucleotide is between about 10 to about 150 nucleotides. In some embodiments, the guide polynucleotide is between about 20 to about 120 nucleotides. In some embodiments, the guide polynucleotide is between about 30 to about 100 nucleotides. In some embodiments, the guide polynucleotide is between about 40 to about 80 nucleotides. In some embodiments, the guide polynucleotide is between about 50 to about 60 nucleotides. In some embodiments, the guide polynucleotide is between about 10 to about 35 nucleotides. In some embodiments, the guide polynucleotide is between about 15 to about 30 nucleotides. In some embodiments, the guide polynucleotide is between about 20 to about 25 nucleotides.

The guide polynucleotide, e.g., guide RNA, can be introduced into the target cell as an isolated molecule, e.g., RNA molecule, or is introduced into the cell using an expression vector containing DNA encoding the guide polynucleotide, e.g., guide RNA.

In some embodiments, the guide polynucleotide of the CRISPR-Cas system is linked to a direct repeat sequence. A direct repeat, or DR, sequence is an array of repetitive sequences in the CRISPR locus, interspaced by short stretches of non-repetitive sequences (spacers). The spacer sequences target the Protospacer Adjacent Motifs (PAM) on the target sequence. When the non-coding portion of the CRISPR locus (i.e., the guide polynucleotide and the tracrRNA) is transcribed, the transcript is cleaved at the DR sequences into short crRNAs containing individual spacer sequences, which direct the Cas9 nuclease to the PAM. In some embodiments, the DR sequence is RNA. In some embodiments, the DR sequence is encoded by a nucleic acid. In some embodiments, the DR sequence is linked to the guide polynucleotide. In some embodiments, the DR sequence is linked to the guide sequence of the guide polynucleotide. In some embodiments, the DR sequence includes a secondary structure. In some embodiments, the DR sequence includes a stem loop structure. In some embodiments, the DR sequence is 10 to 20 nucleotides. In some embodiments, the DR sequence is at least 16 nucleotides. In some embodiments, the DR sequence is at least 16 nucleotides and includes a single stem loop. In some embodiments, the DR sequence includes an RNA aptamer. In some embodiments, the secondary structure or stem loop in the DR is the recognized by a nuclease for cleavage. In some embodiments, the nuclease is a ribonuclease. In some embodiments, the nuclease is RNase III.

In some embodiments, the CRISPR-Cas systems of the present disclosure further include a tracrRNA. A “tracrRNA,” or trans-activating CRISPR-RNA, forms an RNA duplex with a pre-crRNA, or pre-CRISPR-RNA, and is then cleaved by the RNA-specific ribonuclease RNase III to form a crRNA/tracrRNA hybrid. In some embodiments, the guide RNA includes the crRNA/tracrRNA hybrid. In some embodiments, the tracrRNA component of the guide RNA activates the Cas effector protein. In some embodiments, the guide polynucleotide of the CRISPR-Cas system includes a tracrRNA sequence. In some embodiments, the CRISPR-Cas system includes a separate polynucleotide including a tracrRNA sequence.

In some embodiments, the polynucleotide encoding a recombinant Cas effector protein and a guide polynucleotide is on a single vector. In some embodiments, the polynucleotide encoding a recombinant Cas effector protein, a guide polynucleotide (or nucleotide that can be transcribed into a guide polynucleotide), and a tracrRNA are on a single vector. In some embodiments, the polynucleotide encoding a recombinant Cas effector protein, a guide polynucleotide (or nucleotide that can be transcribed into a guide polynucleotide), a tracrRNA, and a direct repeat sequence are on a single vector. In some embodiments, the vector is an expression vector. In some embodiments, the vector is a mammalian expression vector. In some embodiments, the vector is a human expression vector. In some embodiments, the vector is a plant expression vector.

In some embodiments, the polynucleotide encoding a recombinant Cas effector protein and a guide polynucleotide is a single nucleic acid molecule. In some embodiments, the polynucleotide encoding a recombinant Cas effector protein, a guide polynucleotide, and a tracrRNA is a single nucleic acid molecule. In some embodiments, the polynucleotide encoding a recombinant Cas effector protein, a guide polynucleotide, a tracrRNA, and a direct repeat sequence is a single nucleic acid molecule. In some embodiments, the single nucleic acid molecule is an expression vector. In some embodiments, the single nucleic acid molecule is a mammalian expression vector. In some embodiments, the single nucleic acid molecule is a human expression vector. In some embodiments, the single nucleic acid molecule is a plant expression vector.

In some embodiments, the recombinant Cas effector protein and the guide polynucleotide are capable of forming a complex. In some embodiments, the complex of the recombinant Cas effector protein and the guide polynucleotide does not occur in nature.

### Cells

In some embodiments of the disclosure, the eukaryotic cell is a eukaryotic cell. In some embodiments, the eukaryotic cell is an animal or human cell. In some embodiments, the eukaryotic cell is a human or rodent or bovine cell line or cell strain. Examples of such cells, cell lines, or cell strains include, but are not limited to, mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLa, EB1, EB2, EB3, oncolytic or hybridoma-cell lines. In some embodiments, the eukaryotic cells are CHO-cell lines. In some embodiments, the eukaryotic cell is a CHO cell. In some embodiments, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the POTELLIGENT CHOK1 SV (Lonza Biologics, Inc.). Eukaryotic cells can also be avian cells, cell lines or cell strains, such as, for example, EBX cells, EB14, EB24, EB26, EB66, or EBv13.

In some embodiments, the eukaryotic cell is a human cell. In some embodiments, the human cell is a stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In some embodiments, the cell is a pluripotent stem cell. In some embodiments, the cell is an induced pluripotent stem cell. In some embodiments, the human cell is a differentiated form of any of the cells described herein. In some embodiments, the eukaryotic cell is a cell derived from any primary cell in culture.

In some embodiments, the eukaryotic cell is a hepatocyte such as a human hepatocyte, animal hepatocyte, or a non-parenchymal cell. For example, the eukaryotic cell can be a plateable metabolism qualified human hepatocyte, a plateable induction qualified human hepatocyte, plateable human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In some embodiments, the eukaryotic cell is a hematopoietic cell. In some embodiments, the hematopoietic cell is a myeloid progenitor cell. In some embodiments, the hematopoietic cell is a lymphoid progenitor cell. In some embodiments, the hematopoietic cell is a mast cell, a megakarytocyte, a thrombocyte, basophil, a neutrophil, an eosinophil, a dendritic cell, a monocyte, or a macrophage. In some embodiments, the hematopoietic cell is a natural killer cell (NK cell), a T lymphocyte, or a B lymphocyte. In some embodiments, the T or B lymphocyte comprises a chimeric antigen receptor (CAR).

In some embodiments, the eukaryotic cell is a plant cell. For example, the plant cell can be of a crop plant such as cassava, corn, sorghum, wheat, or rice. The plant cell can be of an algae, tree, or vegetable. The plant cell can be of a monocot or dicot or of a crop or grain plant, a production plant, fruit, or vegetable. For example, the plant cell can be of a tree, e.g., a citrus tree such as orange, grapefruit, or lemon tree; peach or nectarine trees; apple or pear trees; nut trees such as almond or walnut or pistachio trees; nightshade plants, e.g., potatoes, plants of the genus Brassica, plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

In some embodiments, the eukaryotic cell is a tissue culture of any of the aforementioned cells. In some embodiments, the eukaryotic cell is in the form of a tissue extract of any of the aforementioned cells.

In some embodiments, the eukaryotic cell comprises a genomically-integrated Cas polynucleotide. In some embodiments, the eukaryotic cell comprises an inducible genomically-integrated Cas polynucleotide.

### Delivery Systems

Various methods are known in the art for delivery of CRISPR-Cas systems. Suitable delivery systems include microinjection, electroporation, transfection, or hydrodynamic delivery of a polynucleotide encoding a Cas effector protein, a polynucleotide comprising a sequence of interest, and/or a polynucleotide capable of forming a complex with a Cas effector protein. In some embodiments, the delivery system comprises a delivery particle. Examples of such delivery systems, including nanoparticles, cell-penetrating peptides, and DNA nanoclews, are disclosed in Lino et al., Drug Delivery, 25 (1): 1234-1257 (2018)).

In some embodiments, the CRISPR-Cas system, including a Cas effector protein, a polynucleotide encoding a Cas effector protein, a polynucleotide encoding a sequence of interest, and/or a polynucleotide capable of forming a complex with a Cas effector protein, of the present disclosure is delivered by a delivery particle. A delivery particle is a biological delivery system or formulation which includes a particle. A “particle,” as defined herein, is an entity having a maximum diameter of about 100 microns (μm). In some embodiments, the particle has a maximum diameter of about 10 μm. In some embodiments, the particle has a maximum diameter of about 2000 nanometers (nm). In some embodiments, the particle has a maximum diameter of about 1000 nm. In some embodiments, the particle has a maximum diameter of about 900 nm, about 800 nm, about 700 nm, about 600 nm, about 500 nm, about 400 nm, about 300 nm, about 200 nm, or about 100 nm. In some embodiments, the particle has a diameter of about 25 nm to about 200 nm. In some embodiments, the particle has a diameter of about 50 nm to about 150 nm. In some embodiments, the particle has a diameter of about 75 nm to about 100 nm.

Delivery particles may be provided in any form, including but not limited to: solid, semi-solid, emulsion, or colloidal particles. In some embodiments, the delivery particle is a lipid-based system, a liposome, a micelle, a microvesicle, an exosome, or a gene gun. In some embodiments, the delivery particle includes a CRISPR-Cas system. In some embodiments, the delivery particle includes a CRISPR-Cas system including a recombinant Cas effector protein and a polynucleotide capable of forming a complex with the Cas effector protein, wherein said polynucleotide comprises a guide polynucleotide. In some embodiments, the delivery particle includes a Cas effector protein, a polynucleotide comprising a sequence of interest, and a polynucleotide capable of forming a complex with a Cas effector protein and comprising a guide polynucleotide. In some embodiments, the delivery particle includes a CRISPR-Cas system including a recombinant Cas effector protein and a polynucleotide which forms a complex with a Cas effector protein and which comprises a guide polynucleotide, wherein the recombinant Cas effector protein and the polynucleotide are in a complex. In some embodiments, the delivery particle includes a CRISPR-Cas system including a recombinant Cas effector protein, a polynucleotide which forms a complex with a Cas effector protein and which comprises a guide polynucleotide, and polynucleotide including a tracrRNA. In some embodiments, the delivery particle includes a CRISPR-Cas system including a Cas effector protein, a polynucleotide which forms a complex with a Cas effector protein and comprises a guide polynucleotide, and a tracrRNA.

In some embodiments, the complex of the Cas effector protein and a polynucleotide of the disclosure is a ribonucleoprotein (RNP), wherein said RNP is delivered via hydrodynamic delivery, a nanoparticle, a vesicle, a cell-penetrating peptide, or a DNA nanoclew.

In some embodiments, the delivery particle further includes a lipid, a sugar, a metal or a protein. In some embodiments, the delivery particle is a lipid envelope. Delivery of mRNA using lipid envelopes or delivery particles including lipids is described, for example, in Su et al., Molecular Pharmacology 8 (3): 774-784 (2011). In some embodiments, the delivery particle is a sugar-based particle, for example, GalNAc. Sugar-based particles are described in WO 2014/118272 and Nair et al., J. Am. Chem. Soc. 136 (49): 16958-16961 (2014).

In some embodiments, the delivery particle is a nanoparticle. Nanoparticles encompassed in the present disclosure may be provided in different forms, e.g., as solid nanoparticles (e.g., metal such as silver, gold, iron, titanium), non-metal, lipid-based solids, polymers, suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present disclosure.

Preparation of delivery particles is further described in U.S. Patent Publications 2011/0293703, 2012/0251560, and 2013/0302401; and U.S. Pat. Nos. 5,543,158, 5,855,913, 5,895,309, 6,007,845, and 8,709,843.

In some embodiments, a vesicle includes the CRISPR-Cas system of the present disclosure. A “vesicle” is a small structure within a cell having a fluid enclosed by a lipid bilayer. In some embodiments, the CRISPR-Cas system of the present disclosure is delivered by a vesicle. In some embodiments, the vesicle includes a recombinant Cas effector protein and a guide polynucleotide. In some embodiments, the vesicle includes a Cas effector protein and a guide polynucleotide, wherein the Cas effector protein and the guide polynucleotide are in a complex. In some embodiments, the vesicle includes a CRISPR-Cas system including a Cas effector protein, a polynucleotide capable of forming a complex with a Cas effector protein and comprising a guide polynucleotide, and a polynucleotide including a tracrRNA. In some embodiments, the vesicle includes a CRISPR-Cas system including a t Cas effector protein, a polynucleotide capable of forming a complex with a Cas effector protein and comprising guide polynucleotide, and a tracrRNA.

In some embodiments, the vesicle including the Cas effector protein and polynucleotide capable of forming a complex with the Cas effector protein and comprising a guide polynucleotide is an exosome or a liposome. In some embodiments, the vesicle is an exosome. In some embodiments, the exosome is used to deliver the CRISPR-Cas systems of the present disclosure. Exosomes are endogenous nano-vesicles (i.e., having a diameter of about 30 to about 100 nm) that transport RNAs and proteins, and which can deliver RNA to the brain and other target organs. Engineered exosomes for delivery of exogenous biological materials into target organs is described, for example, by Alvarez-Erviti et al., Nature Biotechnology 29:341 (2011), El-Andaloussi et al., Nature Protocols 7:2112-2116 (2012), and Wahlgren et al., Nucleic Acids Research 40 (17): e130 (2012).

In some embodiments, the liposome is used to deliver the CRISPR-Cas systems of the present disclosure. Liposomes are spherical vesicle structures having at least one lipid bilayer and can be used as a vehicle for administration of nutrients and pharmaceutical drugs. Liposomes are often composed of phospholipids, in particular phosphatidylcholine, but also other lipids such as egg phosphatidylethanolamine. Types of liposomes include, but are not limited to, multilamellar vesicle, small unilamellar vesicle, large unilamellar vesicle, and cochleate vesicle. See, e.g., Spuch and Navarro, Journal of Drug Delivery, Article ID 469679 (2011). Liposomes for delivery of biological materials such as CRISPR-Cas components are described, for example, by Morrissey et al., Nature Biotechnology 23 (8): 1002-1007 (2005), Zimmerman et al., Nature Letters 441:111-114 (2006), and Li et al., Gene Therapy 19:775-780 (2012).

In some embodiments, the Cas effector protein can be delivered using cell-penetrating peptide fused to the Cas effector protein.

In some embodiments, the Cas effector protein and a polynucleotide of the disclosure can be delivered in the form of a DNA nanoclew. DNA nanoclews are spherical structures comprising DNA that can be loaded with a payload, such as a Cas effector protein (Sun et al., J. Am. Chem. Soc., 136:14722-14725). DNA nanoclews have been used in vitro for delivery of Cas9 editing systems (Lino et al., Drug Delivery, 25 (1): 1234-1257).

In some embodiments, a viral vector includes the CRISPR-Cas systems of the present disclosure. In some embodiments, the CRISPR-Cas system of the present disclosure is delivered by a viral vector. In some embodiments, the viral vector includes a recombinant Cas9 and a guide polynucleotide. In some embodiments, the viral vector includes a Cas effector protein and a guide polynucleotide, wherein the Cas effector protein and the guide polynucleotide are in a complex. In some embodiments, the viral vector includes a CRISPR-Cas system including a Cas effector protein, a polynucleotide capable of forming a complex with a Cas effector protein and comprising a guide polynucleotide, and a polynucleotide including a tracrRNA. In some embodiments, the viral vector includes a CRISPR-Cas system including a Cas effector protein, a polynucleotide capable of forming a complex with a Cas effector protein and comprising a guide polynucleotide, and a tracrRNA. In some embodiments, the viral vector is of a retrovirus, a lentivirus, an adenovirus, or an adeno-associated virus. Examples of viral vectors are provided herein.

In some embodiments, retroviral, lentiviral, adenoviral, and/or adeno-associated virus (AAV) vectors can be used as a viral vector including the elements of the CRISPR-Cas systems as described herein. In some embodiments of the present disclosure, the Cas effector protein is expressed intracellularly by cells transduced by a viral vector.

In some embodiments, the Cas proteins and methods of the present disclosure are used in ex vivo gene editing, such as CAR-T type therapies. These embodiments may involve modification of cells from human donors. In these instances, viral vectors can be also used; however, there is the additional option to directly transfect the Cas9 protein (along with in vitro transcribed guide RNA and donor DNA) into cultured cells.

### Inhibitors of the Micro-Homology End Joining (MMEJ) Pathway

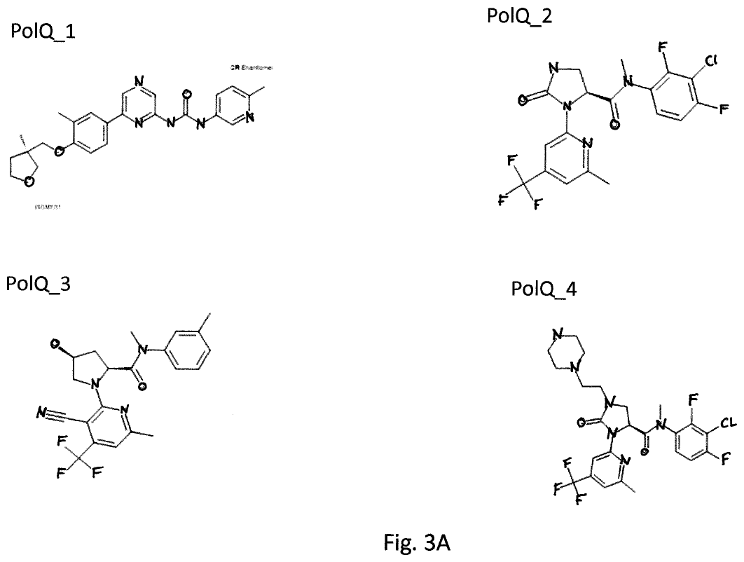

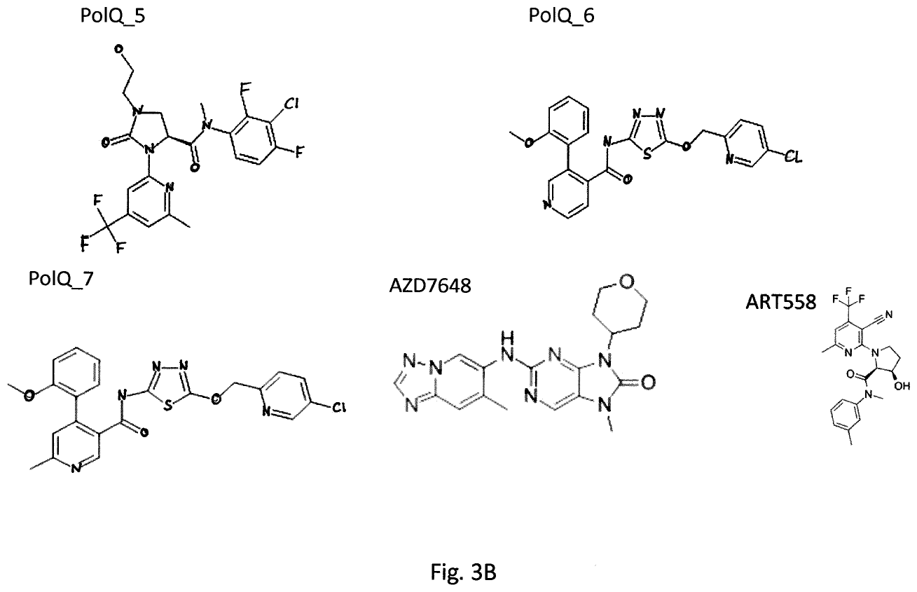

As used herein, an inhibitor of the MMEJ pathway is any compound, molecule, or entity that inhibits, antagonizes, blocks, or decreases the activity and/or level of any component of the MMEJ pathway. The MMEJ inhibitor can be an antibody or antigen-binding fragment thereof, a peptide, soluble protein, siRNA, antisense oligonucleotide, aptamer, or small-molecule compound that inhibits, antagonizes, blocks, or decreases the activity and/or level of any component of the MMEJ pathway. In some embodiments, the MMEJ inhibitor inhibits, antagonizes, blocks, or decreases the activity and/or level of FEN1 (Flap endonuclease 1), DNA ligase III, MREII, NBS1 (Nibrin, NBN), XRCC1 (X-ray repair cross-complementing protein 1), PARP1 (Poly [ADP-ribose] polymerase 1), or PolQ (DNA polymerase θ). In some embodiments, the inhibitor of the MMEJ pathway is novobiocin. In some embodiments, the inhibitor of the MMEJ pathway is a PolQ inhibitor. In some embodiments, the PolQ inhibitor is ART558 (Zatreanu et al., Nature Communications, 12 (1): 3636 (2021)). In some embodiments, the PolQ inhibitor is selected from PolQ 1 (as described in WO2020030925), PolQ2, PolQ3, PolQ4, PolQ5 (all as described in WO 2021028643), PolQ6, PolQ7 (as described in WO2020243549), or combinations thereof, as shown in FIG. 3.

In some embodiments, the inhibitor of the MMEJ pathway is added to the composition comprising the eukaryotic cell at a concentration of about 0.01 μM to about 1 mM. In some embodiments the concentration of the inhibitor of the MMEJ pathway is about 0.01 μM to about 0.75 mM, about 0.01 μM to about 0.5 mM, about 0.01 μM to about 0.25 mM, about 0.01 μM to about 0.1 mM, about 0.01 μM to about 75 M, about 0.01 μM to about 50 μM, about 0.01 μM to about 25 μM, about 0.01 to about 25 μM, about 0.01 to about 20 μM, about 0.01 μM to about 15 μM, about 0.01 μM to about 10 μM, or about 0.01 μM to about 1 μM. In some embodiments the concentration of the inhibitor of the MMEJ pathway is about 0.1 μM to about 1 mM, about 1 μM to about 1 mM, about 10 μM to about 1 mM, about 15 μM to about 1 M, about 20 μM to about 1 M, about 25 μM to about 1 mM, about 50 μM to about 1 mM, about 75 μM to about 1 mM, about 0.1 mM to about 1 mM, about 0.25 mM to about 1 mM, about 0.5 mM to about 1 mM, or about 0.75 mM to about 1 mM. In some embodiments, the concentration of the inhibitor of the MMEJ pathway is about 0.1 μM to about 1 mM, 0.1 μM to about 0.75 mM, about 0.1 μM to about 0.5 mM, about 0.1 μM to about 0.25 mM, about 0.1 μM to about 0.1 mM, about 0.1 μM to about 75 μM, about 0.1 μM to about 50 μM, about 0.1 μM to about 25 μM, about 0.1 μM to about 20 μM, about 0.1 μM to about 15 μM, about 0.1 μM to about 10 μM, or about 0.1 μM to about 1 μM. In some embodiments, the concentration of the inhibitor of the MMEJ pathway is about 1 μM to about 10 μM, about 1 μM to about 15 μM, about 1 μM to about 20 μM, about 1 μM to about 25 μM, about 1 μM to about 50 μM, about 1 μM to about 0.1 mM, about 1 μM to about 0.25 mM, about 1 μM to about 0.5 mM, about 1 μM to about 0.75 mM, or about 1 μM to about 1 mM. In some embodiments, the concentration of the inhibitor of the MMEJ pathway is about 0.01 μM to about 100 μM, about 0.1 μM to about 90 μM, about 0.2 μM to about 80 μM, about 0.3 μM to about 70 μM, about 0.4 μM to about 60 μM, about 0.5 μM to about 50 μM, about 1 μM to about 50 μM, about 2 μM to about 45 μM, about 3 μM to about 40 μM, about 4 μM to about 35 μM, about 5 μM to about 30 μM, about 6 μM to about 25 μM, about 7 μM to about 20 μM, or about 8 μM to about 15 μM. In some embodiments, the concentration of the inhibitor of the MMEJ pathway is about 0.01 μM to about 0.1 μM, about 0.01 to about 1 μM, about 0.05 μM to about 0.1 μM, about 0.5 μM to about 1 μM, about 0.5 μM to about 5 μM, about 0.5 μM to about 10 μM, about 0.1 μM to about 1 μM, about 0.1 μM to about 5 μM, about 0.1 μM to about 10 μM, about 1 μM to about 5 μM, about 1 μM to about 10 μM, about 1 μM to about 15 μM, about 1 μM to about 20 μM, about 1 μM to about 25 μM, about 1 μM to about 50 μM, about 5 μM to about 10 μM, about 5 μM to about 15 μM, about 5 mM to about 20 mM, or about 5 mM to about 25 mM. In some embodiments, the concentration of the inhibitor of the MMEJ pathway is about 0.1, 0.2, 0.3, 0.4, 0.5, 0.6, 0.7, 0.7, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 μM.

In some embodiments, the concentration of the inhibitor of the MMEJ pathway is 0.01 μM to about 1 μM, about 0.1 μM to about 1 μM, about 0.1 μM to about 0.5 μM, about 0.1 μM to about 100 μM, or about 1 μM to about 50 μM.

In some embodiments, the inhibitor of the MMEJ pathway is added to the composition comprising the eukaryotic cell about 0 minutes to about 96 hours before the Cas effector protein is added, about 0 minutes to about 72 hours before the Cas effector protein is added, about 0 minutes to about 48 hours before the Cas effector protein is added, about 0 minutes to about 36 hours before the Cas effector protein is added, about 0 minutes to about 24 hours before the Cas effector protein is added, about 0 minutes to about 18 hours before the Cas effector protein is added, about 0 minutes to about 12 hours before the Cas effector protein is added, about 0 minutes to about 6 hours before the Cas effector protein is added, about 0 minutes to about 3 hours before the Cas effector protein is added, about 0 minutes to about 2 hours before the Cas effector protein is added, about 0 minutes to about 1 hour before the Cas effector protein is added, or about 0 minutes to about 30 minutes before the Cas effector protein is added. In some embodiments, the inhibitor of the MMEJ pathway is added to the composition comprising a eukaryotic cell about 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, 30, 31, 32, 33, 34, 35, 40, 45, 50, 55, 60, 65, 70, 75, 80, 85, 90, 95, or 100 hours before the Cas effector protein is added.

In some embodiments, the inhibitor of the MMEJ pathway is added to the composition comprising a eukaryotic cell at the same time the Cas effector protein is added.

In some embodiments, the inhibitor of the MMEJ pathway is added to the composition comprising a eukaryotic cell about 0 minutes to about 30 minutes after the Cas effector protein is added, about 0 minutes to about 1 hour after the Cas effector protein is added, about 0 minutes to about 3 hours after the Cas effector protein is added, about 0 minutes to about 6 hours after the Cas effector protein is added, about 0 minutes to about 12 hours after the Cas effector protein is added, about 0 minutes to about 18 hours after the Cas effector protein is added, about 0 minutes to about 24 hours after the Cas effector protein is added, about 0 minutes to about 36 hours after the Cas effector protein is added, about 0 minutes to about 48 hours after the Cas effector protein is added, about 0 minutes to about 72 hours after the Cas effector protein is added, or about 0 minutes to about 96 hours after the Cas effector protein is added. In some embodiments, the inhibitor of the MMEJ pathway is added to the composition comprising a eukaryotic cell about 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, 30, 31, 32, 33, 34, 35, 40, 45, 50, 55, 60, 65, 70, 75, 80, 85, 90, 95, or 100 hours after the Cas effector protein is added.

In some embodiments, the inhibitor of the MMEJ pathway is in the composition comprising a eukaryotic cell for about 1 to about 300 hours, about 10 to about 200 hours, about 10 to about 100 hours, about 20 to about 80 hours, about 30 to about 70 hours, or about 40 to about hours. In some embodiments, the inhibitor of the MMEJ pathway is in the composition comprising a eukaryotic cell for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100, 125, 150, 175, 200, 225, 250, 275, or 300 hours.

In some embodiments, the inhibitor of the MMEJ pathway is added to the composition comprising a eukaryotic cell at least 1, 2, 3, 4, 5, 6, 7, 8, 9, or 10 or more times.

### Inhibitors of the Non-Homologous End Joining (NHEJ) Pathway

As used herein, an inhibitor of the NHEJ pathway is any compound, molecule, or entity that inhibits, antagonizes, blocks, or decreases the activity and/or level of any component of the NHEJ pathway. The NHEJ inhibitor can be an antibody or antigen-binding fragment thereof, a peptide, soluble protein, siRNA, antisense oligonucleotide, aptamer, or small-molecule compound that inhibits, antagonizes, blocks, or decreases the activity and/or level of any component of the NHEJ pathway. In some embodiments, the NHEJ pathway inhibits, antagonizes, blocks, or decreases the activity and/or level of Ku70, Ku80, DNA Ligase IV, XLF (non-homologous end-joining factor 1; XRCC4-like factor), or DNA-dependent protein kinase (DNA-PK). In some embodiments, the inhibitor of DNA-PK is M3814, M9831/VX984, Nu7441, KU0060648, AZD7648, Nu5455, vanillin, wortmannin, or combinations thereof. In some embodiments, the inhibitor of DNA-PK is AZD7648.

In some embodiments, the inhibitor of the NHEJ pathway is added to the composition comprising the eukaryotic cell at a concentration of about 0.01 μM to about 1 mM. In some embodiments the concentration of the inhibitor of the NHEJ pathway is about 0.01 μM to about 0.75 mM, about 0.01 μM to about 0.5 mM, about 0.01 μM to about 0.25 mM, about 0.01 μM to about 0.1 mM, about 0.01 μM to about 75 μM, about 0.01 μM to about 50 μM, about 0.01 μM to about 25 μM, about 0.01 to about 25 μM, about 0.01 to about 20 μM, about 0.01 μM to about 15 μM, about 0.01 μM to about 10 μM, or about 0.01 μM to about 1 μM. In some embodiments the concentration of the inhibitor of the NHEJ pathway is about 0.1 μM to about 1 mM, about 1 μM to about 1 mM, about 10 μM to about 1 mM, about 15 μM to about 1 M, about 20 μM to about 1 M, about 25 μM to about 1 mM, about 50 μM to about 1 mM, about 75 μM to about 1 mM, about 0.1 mM to about 1 mM, about 0.25 mM to about 1 mM, about 0.5 mM to about 1 mM, or about 0.75 mM to about 1 mM. In some embodiments, the concentration of the inhibitor of the NHEJ pathway is about 0.1 μM to about 1 mM, 0.1 μM to about 0.75 mM, about 0.1 μM to about 0.5 mM, about 0.1 μM to about 0.25 mM, about 0.1 μM to about 0.1 mM, about 0.1 μM to about 75 μM, about 0.1 μM to about 50 μM, about 0.1 μM to about 25 μM, about 0.1 μM to about 20 μM, about 0.1 μM to about 15 M, about 0.1 μM to about 10 μM, or about 0.1 μM to about 1 μM. In some embodiments, the concentration of the inhibitor of the NHEJ pathway is about 1 μM to about 10 μM, about 1 μM to about 15 μM, about 1 μM to about 20 μM, about 1 μM to about 25 μM, about 1 μM to about 50 μM, about 1 μM to about 0.1 mM, about 1 μM to about 0.25 mM, about 1 μM to about 0.5 mM, about 1 μM to about 0.75 mM, or about 1 μM to about 1 mM. In some embodiments, the concentration of the inhibitor of the NHEJ pathway is about 0.01 μM to about 100 μM, about 0.1 μM to about 90 μM, about 0.2 μM to about 80 μM, about 0.3 μM to about 70μ, about 0.4μ M to about 60μ, about 0.5μ M to about 50μ, about 1μ M to about 50μ M, about 2 μM to about 45 μM, about 3 μM to about 40 μM, about 4 μM to about 35 μM, about 5 μM to about 30 μM, about 6 μM to about 25 μM, about 7 μM to about 20 μM, or about 8 μM to about 15 μM. In some embodiments, the concentration of the inhibitor of the NHEJ pathway is about 0.01 μM to about 0.1 μM, about 0.01 to about 1 μM, about 0.05 μM to about 0.1 μM, about 0.5 μM to about 1 μM, about 0.5 μM to about 5 μM, about 0.5 μM to about 10 μM, about 0.1 μM to about 1 μM, about 0.1 μM to about 5 μM, about 0.1 μM to about 10 μM, about 1 μM to about 5 μM, about 1 μM to about 10 μM, about 1 μM to about 15 μM, about 1 μM to about 20 M, about 1 μM to about 25 μM, about 1 μM to about 50 μM, about 5 μM to about 10 μM, about 5 μM to about 15 μM, about 5 mM to about 20 mM, or about 5 mM to about 25 mM. In some embodiments, the concentration of the inhibitor of the NHEJ pathway is about 0.1, 0.2, 0.3, 0.4, 0.5, 0.6, 0.7, 0.7, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 μM.

In some embodiments, the concentration of the inhibitor of the NHEJ pathway is 0.01 μM to about 1 μM, about 0.1 μM to about 1 μM, about 0.1 μM to about 0.5 μM, about 0.1 μM to about 100 μM, or about 1 μM to about 50 μM.

In some embodiments, the inhibitor of the NHEJ pathway is added to the composition comprising the eukaryotic cell about 0 minutes to about 96 hours before the Cas effector protein is added, about 0 minutes to about 72 hours before the Cas effector protein is added, about 0 minutes to about 48 hours before the Cas effector protein is added, about 0 minutes to about 36 hours before the Cas effector protein is added, about 0 minutes to about 24 hours before the Cas effector protein is added, about 0 minutes to about 18 hours before the Cas effector protein is added, about 0 minutes to about 12 hours before the Cas effector protein is added, about 0 minutes to about 6 hours before the Cas effector protein is added, about 0 minutes to about 3 hours before the Cas effector protein is added, about 0 minutes to about 2 hours before the Cas effector protein is added, about 0 minutes to about 1 hour before the Cas effector protein is added, or about 0 minutes to about 30 minutes before the Cas effector protein is added. In some embodiments, the inhibitor of the NHEJ pathway is added to the composition comprising a eukaryotic cell about 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, 30, 31, 32, 33, 34, 35, 40, 45, 50, 55, 60, 65, 70, 75, 80, 85, 90, 95, or 100 hours before the Cas effector protein is added.

In some embodiments, the inhibitor of the NHEJ pathway is added to the composition comprising a eukaryotic cell at the same time the Cas effector protein is added.

In some embodiments, the inhibitor of the NHEJ pathway is added to the composition comprising a eukaryotic cell about 0 minutes to about 30 minutes after the Cas effector protein is added, about 0 minutes to about 1 hour after the Cas effector protein is added, about 0 minutes to about 3 hours after the Cas effector protein is added, about 0 minutes to about 6 hours after the Cas effector protein is added, about 0 minutes to about 12 hours after the Cas effector protein is added, about 0 minutes to about 18 hours after the Cas effector protein is added, about 0 minutes to about 24 hours after the Cas effector protein is added, about 0 minutes to about 36 hours after the Cas effector protein is added, about 0 minutes to about 48 hours after the Cas effector protein is added, about 0 minutes to about 72 hours after the Cas effector protein is added, or about 0 minutes to about 96 hours after the Cas effector protein is added. In some embodiments, the inhibitor of the NHEJ pathway is added to the composition comprising a eukaryotic cell about 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, 30, 31, 32, 33, 34, 35, 40, 45, 50, 55, 60, 65, 70, 75, 80, 85, 90, 95, or 100 hours after the Cas effector protein is added.

In some embodiments, the inhibitor of the NHEJ pathway is in the composition comprising a eukaryotic cell for about 1 to about 300 hours, about 10 to about 200 hours, about 10 to about 100 hours, about 20 to about 80 hours, about 30 to about 70 hours, or about 40 to about hours. In some embodiments, the inhibitor of the NHEJ pathway is in the composition comprising a eukaryotic cell for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100, 125, 150, 175, 200, 225, 250, 275, or 300 hours.

In some embodiments, the inhibitor of the NHEJ pathway is added to the composition comprising a eukaryotic cell at least 1, 2, 3, 4, 5, 6, 7, 8, 9, or 10 or more times.

In some embodiments, the inhibitor of the NHEJ pathway is added to the composition comprising a eukaryotic cell before the inhibitor of the MMEJ pathway is added to the composition comprising a eukaryotic cell after the inhibitor of the MMEJ pathway is added to the composition. In some embodiments, the inhibitor of the NHEJ pathway and the inhibitor of the MMEJ pathway are added to the composition comprising a eukaryotic cell at the same time.

In some embodiments, the inhibitor of the MMEJ pathway and the inhibitor of the NHEJ pathway are added to the composition comprising a eukaryotic cell before the Cas effector protein is added. In some embodiments, the inhibitor of the MMEJ pathway and the inhibitor of the NHEJ pathway are added to the composition comprising a eukaryotic cell after the Cas effector protein is added. In some embodiments, the inhibitor of the MMEJ pathway and the inhibitor of the NHEJ pathway are added to the composition comprising a eukaryotic cell at the same time the Cas effector protein is added. In some embodiments, the inhibitor of the MMEJ pathway is added to the composition comprising a eukaryotic cell before the Cas effector protein is added and the inhibitor of the NHEJ pathway is added after the Cas effector protein is added. In some embodiments, the inhibitor of the MMEJ pathway is added to the composition comprising a eukaryotic cell after the Cas effector protein is added and the inhibitor of the NHEJ pathway is added before the Cas effector protein is added.

All references cited herein, including patents, patent applications, papers, textbooks and the like, and the references cited therein, to the extent that they are not already, are hereby incorporated herein by reference in their entirety.

### EXAMPLES

### Example 1—Effect of MMEJ and NHEJ Inhibitors on Double-Strand Break Repair Pathways

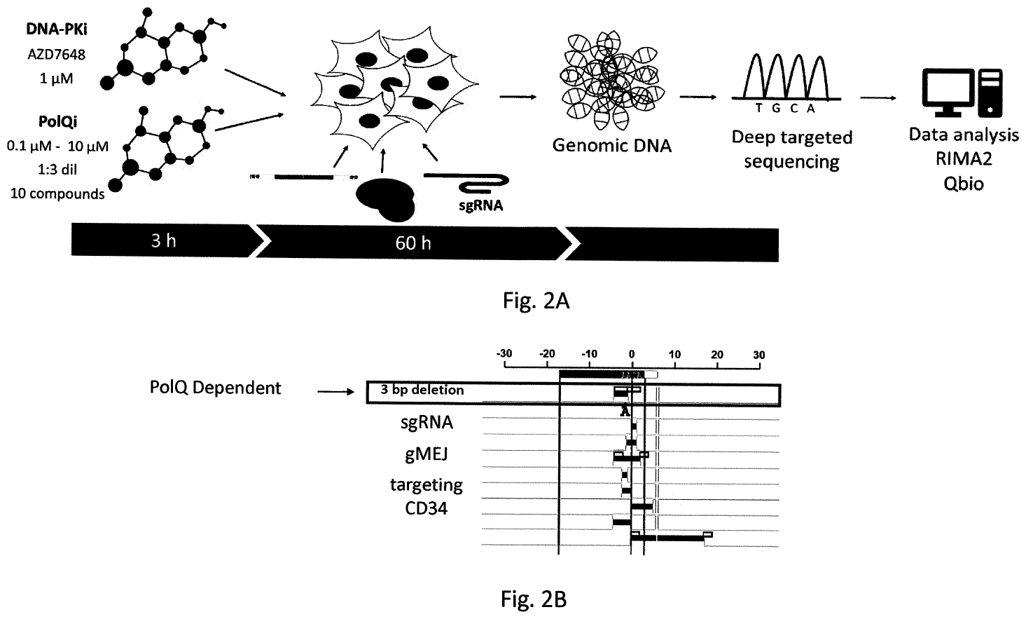

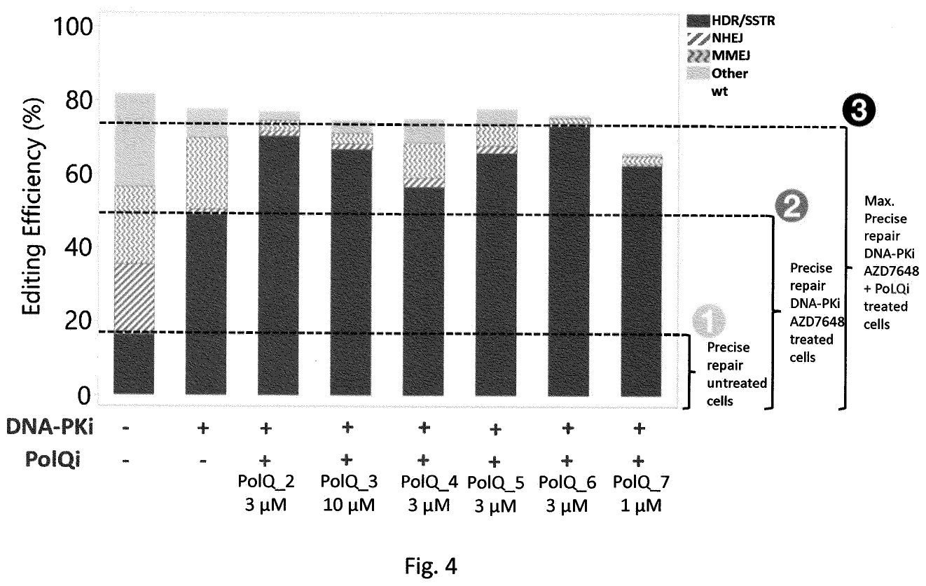

The effect of inhibitors of the MMEJ and NHEJ pathways on CRISPR-Cas-induced DNA double stranded break repair pathways was examined using the process shown schematically in FIG. 2A. Briefly, HEK293T cells were seeded into a 96-well plate 20 hours before transfection with plasmids encoding SpCas9 and a guide RNA (sgRNA) targeting CD34 together with a single-stranded oligonucleotide donor (ssDNA). Three hours prior to transfection, the cells were treated with the DNA-dependent protein kinase (DNA-PK) inhibitor AZD7648 (an NHEJ inhibitor) at a final concentration of 1 μM, alone and in combination with 6 different Pol Q inhibitors (MMEJ inhibitors) at the concentrations indicated in FIG. 4. The Pol Q inhibitors used are PolQ_2, PolQ_3, PolQ_4, PolQ_5, PolQ_6 or PolQ_7. Sixty hours post-transfection, genomic DNA was harvested and sequenced using deep-targeted amplicon sequencing. Genetic variants of the sequencing data were determined using a bioinformatic workflow, and the percentages of DNA double-stranded break repair by the MMEJ, NHEJ, and HDR pathways were determined using Rational InDel Meta-Analysis (RIMA). See, e.g., Taheri-Ghafarokhi et al, Nuc. Acids Res., 2018, 46 (16): 8417-8434. The RIMA results are plotted as shown in in FIG. 2B, where deletions associated with microhomologies are visualized according to the bars shown in the figure.

The results of these experiments are shown in FIG. 4. In transfected cells not treated with MMEJ or NHEJ inhibitors (DMSO treated controls), approximately 20% of double-stranded breaks were repaired by the HDR pathway, while the NHEJ and MMEJ pathways were responsible for approximately 40% of double-stranded break repair. In contrast, transfected cells treated with an NHEJ inhibitor (AZD7648) alone or in combination an MMEJ inhibitor (Pol Q 2-7) demonstrated a marked in increase in double-strand break repair by the HDR pathway, while repair by the MMEJ and NHEJ pathways was decreased. Treatment of transfected cells with both NHEJ and MMEJ inhibitors resulted in repair of most double strand breaks via the HDR pathway, and in some instances, nearly all of the double strand break repair was via the HDR pathway.

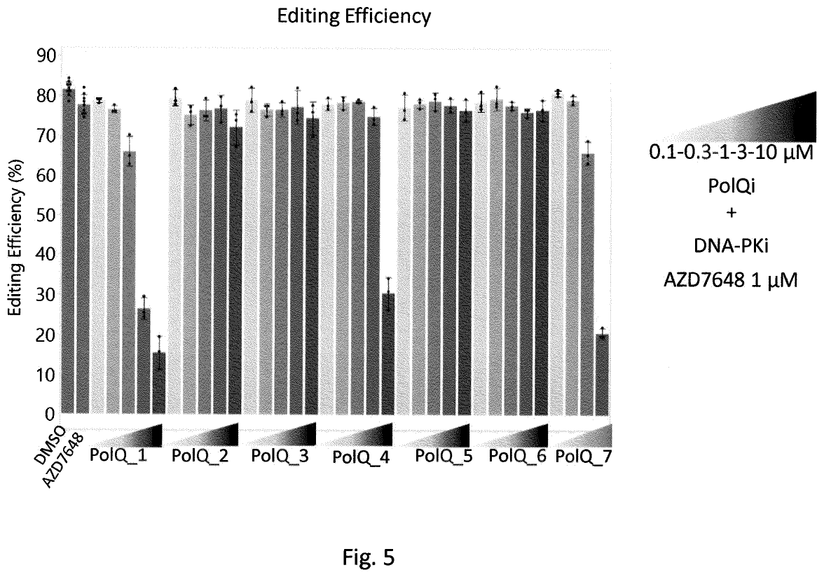

To demonstrate the effect of various inhibitors on CRISPR/Cas editing efficiency, HEK293T cells were treated with the DNA-PK inhibitor AZD7648 (1 μM) alone and in combination with the indicated Pol Q inhibitors, followed by CRISPR/Cas9-mediated gene targeting. As shown in FIG. 5, the NHEJ inhibitor and most concentrations of MMEJ inhibitors used in these experiments did not affect the CRISPR/Cas-mediated editing efficiency. These studies show that inhibition of NHEJ and/or MMEJ pathways in combination with CRISRP/Cas-gene targeting results in DNA double strand break repair by the more precise HDR pathway, and minimizes the contribution from the more error-prone MMEJ and NHEJ pathways.

### Example 2—Effect of MMEJ and NHEJ Inhibitors on CRISPR/Cas-Mediated Knock-In Efficiency

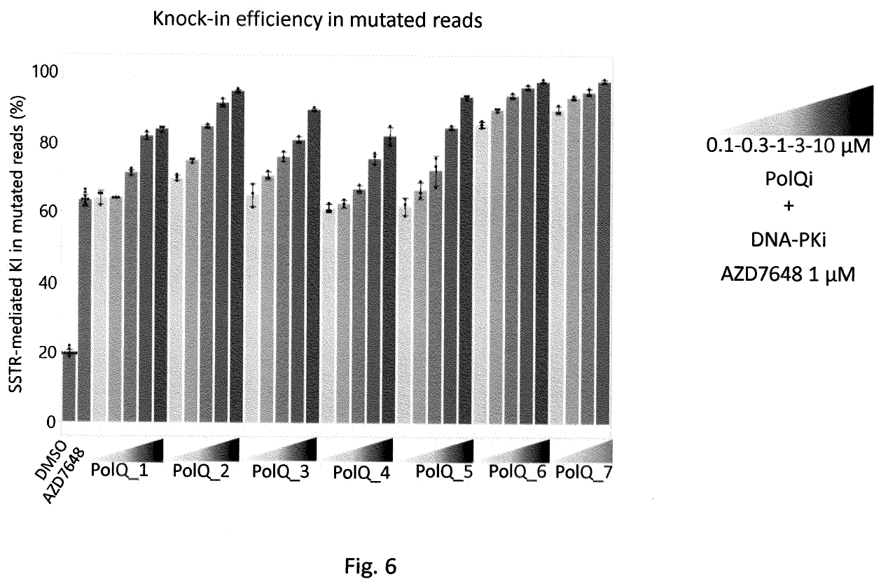

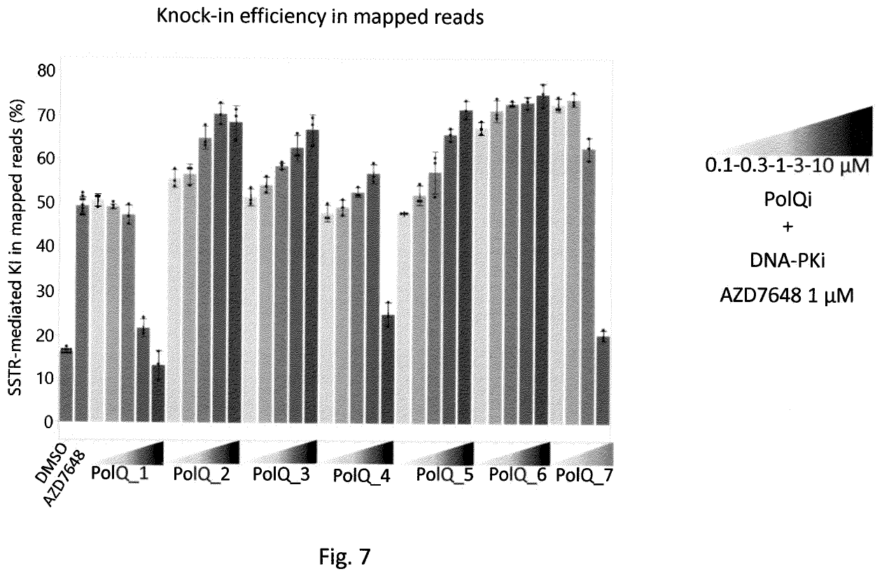

The effect of NHEJ and MMEJ inhibitors on the CRISPR/Cas-mediated knock-in efficiency was determined in both mutated and mapped reads. Briefly, HEK293T cells were cultured and transfected, and then treated with an NHEJ inhibitor (AZD7648) alone and in combination with MMEJ inhibitors (Pol Q 1-7) following the protocol described in Example 1, followed by isolation of genomic DNA and subsequent analysis of knock-in efficiency in both mutated and mapped reads. Inhibition of the NHEJ and MMEJ pathways resulted in an approximately 3-fold increase in knock-in events compared to DMSO-treated controls when assessing both mutated (FIG. 6) and mapped (FIG. 7) reads. Inhibition of the MMEJ pathway in combination with inhibition of the NHEJ pathway increased knock-in efficiencies up to 4.5-fold in the total cell population, and up to 5.9-fold in CRISPR/Cas-edited cells.

### Example 3—Effect of MMEJ Inhibition on Mutated and Mapped Reads

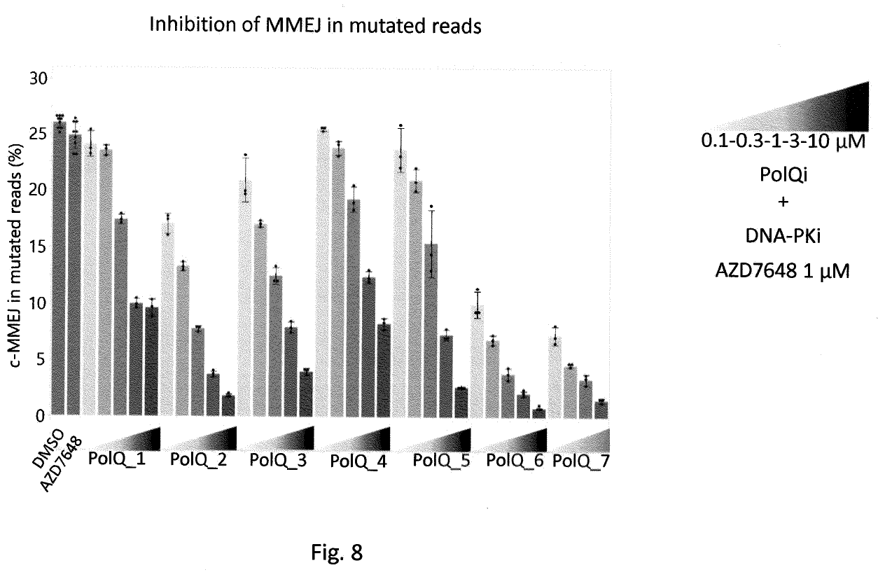

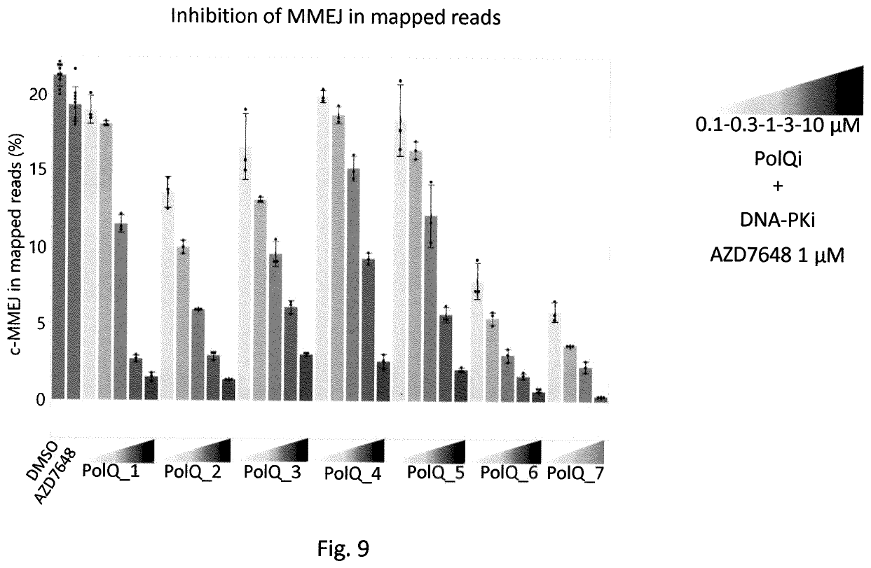

HEK293T cells were cultured, transfected, and treated with the DNA-PK inhibitor AZD7648 (1 μM) alone and in combination with the indicated Pol Q inhibitors, followed by CRISPR/Cas9-mediated gene knock-in. The effect of MMEJ pathway inhibition on mutated and mapped reads was assessed. Treatment of CRISPR/Cas-edited cells with MMEJ inhibitors resulted in a dose-dependent decrease in MMEJ-mutated reads (FIG. 8) and MMEJ-mapped reads (FIG. 9).

### Example 4—Inhibition of the NHEJ and MMEJ Pathways does not Affect Cell Confluency and Transfection Efficiency in CRISPR/Cas-Transfected Cells

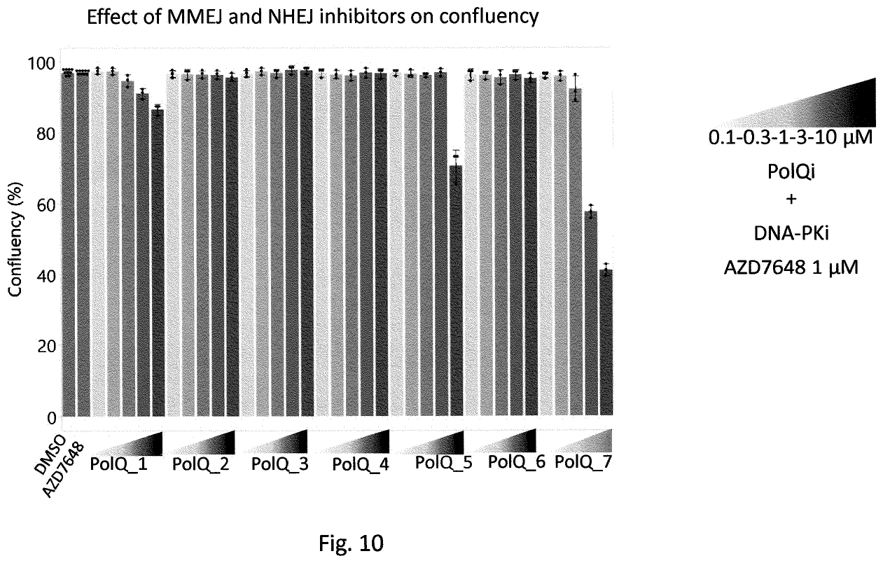

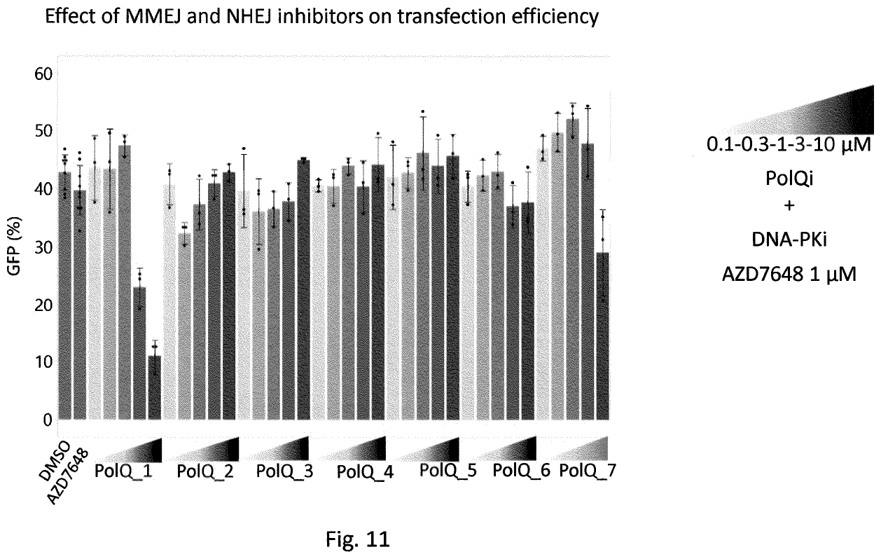

HEK293T cells were cultured, transfected, and treated with NHEJ and MMEJ inhibitors as described in Example 1. Cell confluency and transfection efficiency was assessed in transfected cells treated with NHEJ and MMEJ inhibitors. As shown in FIG. 10, treating transfected cells with the NHEJ inhibitor (AZD7648) at a final concentration of 1 mM had no significant effect on cell confluency. Treating the transfected cells with the NHEJ inhibitor in combination with the indicated Pol Q inhibitors had no effect on cell confluency except at the highest concentrations of PolQ_1, PolQ_5, and PolQ_7. Similarly, as show in FIG. 11, the treating the cells with the NHEJ inhibitor (AZD7648) at 1 μM prior to transfection had no significant effect on the transfection efficiency. Treating the cells with the NHEJ inhibitor in combination with the indicated PolQ inhibitors prior to transfection had no effect on transfection efficiency except at the highest concentrations of PolQ_1 and PolQ_7.

### Example 5—Effect of NHEJ and MMEJ Inhibitors on Double-Strand Break Repair Pathways in Induced Pluripotent Stem Cells (iPSCs)

The effect of NHEJ and/or MMEJ pathway inhibition on CRISPR-Cas-induced DNA double stranded break repair pathways in iPSCs was examined. Briefly, iPSCs comprising an inducible Cas9 gene were seeded into a 96-well plate 20 hours before transfection with a plasmid encoding a guide RNA (sgRNA) targeting one of three separate target sites together with a single-stranded oligonucleotide donor (ssDNA), followed by induction of Cas9 expression. Three hours prior to transfection and induction of Cas9 expression, the iPSCs were treated with the DNA-dependent protein kinase (DNA-PK) inhibitor AZD7648 at a final concentration of 1 μM, alone and in combination with PolQ 2 or PolQ 6 at 3 μM. Sixty hours post-transfection, the percentage of double-stranded break repair by the HDR, NHEJ, and MMEJ pathways was determined as discussed in Example 1.

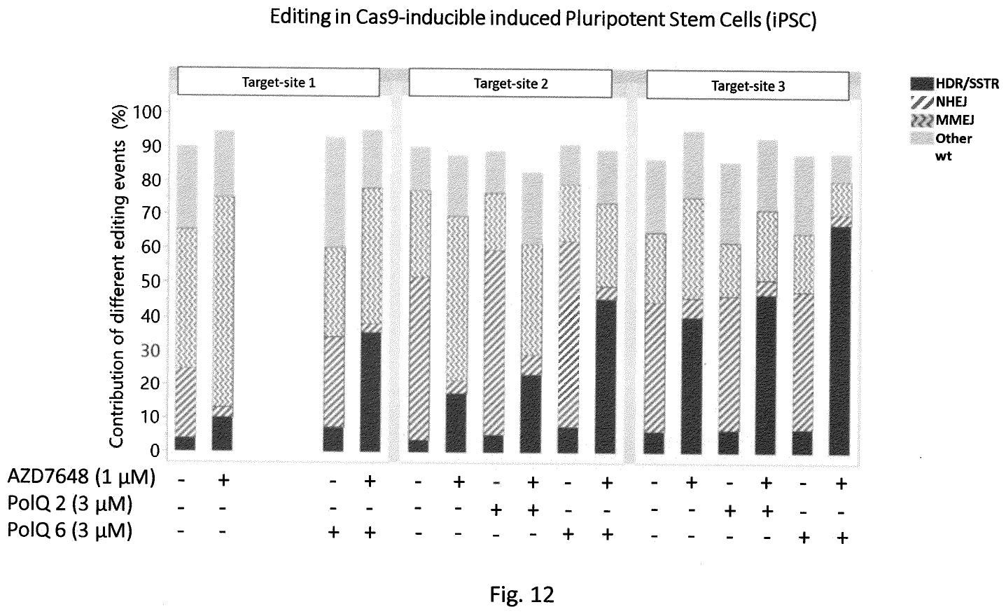

The results of these experiments are shown in FIG. 12. In transfected cells not treated with MMEJ or NHEJ inhibitors (DMSO treated controls), less than 10% of double-stranded breaks were repaired by the HDR pathway, while the NHEJ and MMEJ pathways were responsible for approximately 70% of double-stranded break repair. In contrast, transfected cells treated with an NHEJ inhibitor (AZD7648) or an MMEJ inhibitor (Pol Q 2 or Pol Q 6) demonstrated a marked in increase in double-strand break repair by the HDR pathway, while repair by the MMEJ and NHEJ pathways was decreased. Combined treatment with the NHEJ inhibitor and an MMEJ inhibitor resulted in an even greater increase in HDR-mediated repair and corresponding decrease in NHEJ- and MMEJ-mediated repair.

### Example 6—Effect of NHEJ and MMEJ Inhibitors on Single-Strand Template Repair (SSTR) Gene Insertion in iPSCs

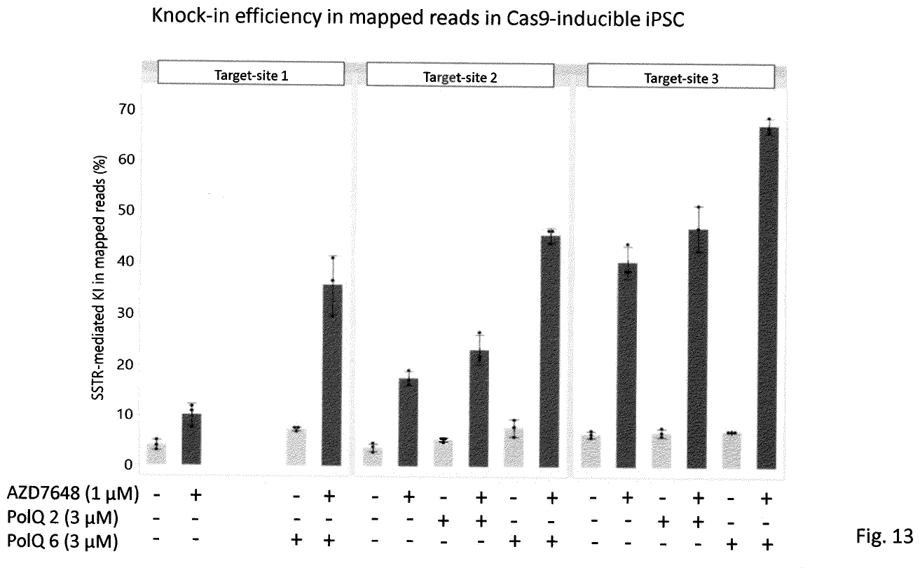

The effect of NHEJ and MMEJ pathway inhibition on gene knock-in efficiency mediated by the SSTR pathway in iPSCs was investigated. Briefly, Cas9-inducible iPSCs were cultured and transfected with sgRNA and ssDNA polynucleotides as described in Example 5. As shown in FIG. 13, transfected cells not treated with NHEJ or MMEJ inhibitors (DMSO treated controls), the SSTR pathway contributed to less than 5% of gene knock-in mapped reads at 3 separate target sites. Addition of the NHEJ inhibitor AZD7648 increased SSTR-mediated gene knock-ins at all three target sites. Similarly, addition of the MMEJ inhibitors PolQ 2 or PolQ6 also increased SSTR-mediated gene knock-in at all three target sites. Combined addition of the NHEJ inhibitor and MMEJ inhibitors significantly increased SSTR-mediated gene knock-in at all three target sites.

### Example 7—Effect of NHEJ and MMEJ Inhibitors on Gene Insertion in Primary Human T Cells

The effect of NHEJ and MMEJ pathway inhibition on gene insertion in human primary T cells was investigated. Briefly, human T cells were treated with the NHEJ inhibitor AZD7648 at 1 μM, alone or in combination with the MMEJ inhibitors PolQ 2 or PolQ6 at 3 μM. Three hours later, the cells were transfected with a ribonucleoprotein (RNP) comprising Cas9 and a sgRNA targeting TRAC, and a polynucleotide encoding green fluorescent protein (GFP). Sixty hours post-transfection, GFP knock-in efficiency was determined as described in Example 1.

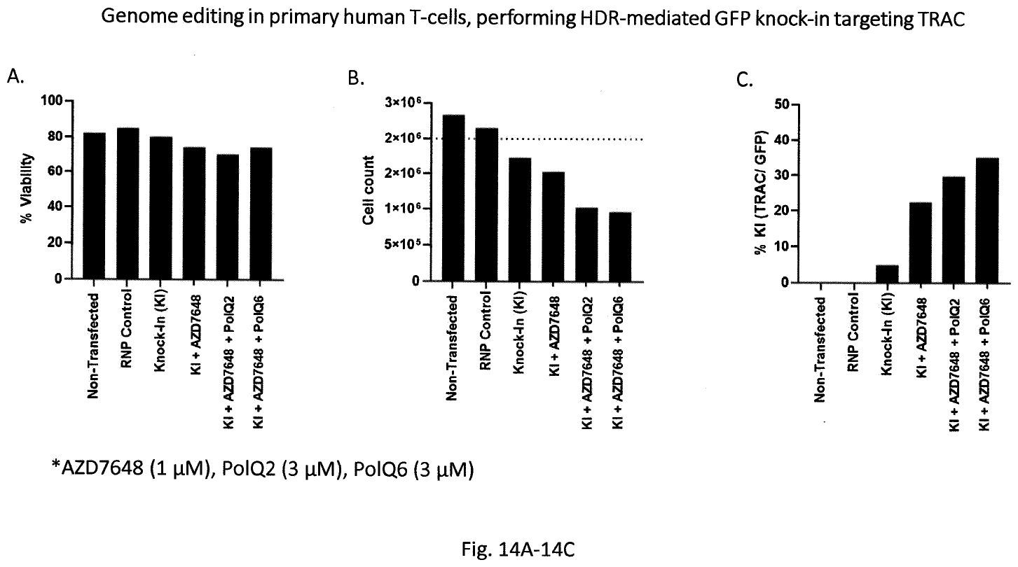

The results of these experiments are shown in FIGS. 14A-C. Transfection of primary T cells and treatment with NHEJ and MMEJ inhibitors had no effect on cell viability (FIG. 14A), and resulted in a moderate reduction in cell number (FIG. 14B). Transfected primary human T cells which were not treated with NHEJ or MMEJ inhibitors exhibited approximately 5% GFP knock-in efficiency. However, the GFP knock-in efficiency was significantly increased by treatment with the NHEJ inhibitor, either alone or in combination with MMEJ inhibitors (FIG. 14C). Notably, knock-in efficiency was significantly enhanced by combined NHEJ and MMEJ pathway inhibition.

### Example 8—Effect of Different DNAPK Inhibitors on Precise Gene Editing Outcomes

HEK293T cells were seeded into 96-well plates containing media and including the following conditions: a) DMSO b) 0.3125, 0.625, 1.25, 2.5, 10 μM DNAPK inhibitor TLR1 (ISAC: (4-fluoro-3-(7-morpholinoquinazolin-4-yl)phenyl) (3-methylpyrazin-2-yl) methanol surechembl: SCHEMBL16235486) c) 0.3125, 0.625, 1.25, 2.5, 10 μM DNAPK inhibitor TLR2 (ISAC: 5-methyl-2-((7-methyl-[1,2,4]triazolo[1,5-a]pyridin-6-yl)amino)-8-(tetrahydro-2H-pyran-4-yl)-7,8 dihydropteridin-6 (5H)-one MedChem ELN: ELNC025305144) d) 0.3125, 0.625, 1.25, 2.5, 10 μM DNAPK inhibitor M9831/VX-984 e) 0.3125, 0.625, 1.25, 2.5, 10 μM DNAPK inhibitor AZD7648. Cells allowed to attach for 12 hours before transfection. Cells were transfected with DNA plasmids encoding for SpCas9-EGFP and a sgRNAs targeting CD34 (gINS) in the presence of single-stranded oligonucleotide donor (ssDNA). 70 hours post-transfection cell confluence and EGFP-based transfection efficiencies were determined with the Incucyte S3. Genomic DNA was extracted and editing outcome was analysed through deep-targeted amplicon sequencing using bioinformatic analysis.

As illustrated by the data in Table 2 below, all tested DNAPK inhibitors increase precise knock-in frequencies of the provided single-stranded oligonucleotide donor and decrease unprecise DNA repair events from NHEJ in a concentration-dependent manner with similar efficiencies.

TABLE 2

| | | | | | | | | | | Precise | |
|---|---|---|---|---|---|---|---|---|---|---|---|
| | DNAPK | | | | | | | | | KI | HDR to |
| | inhibitor | Total editing | | NHEJ indels | | MMEJ indels | | Precise KI | | fold | indel |
| Treatment | (μM) | Mean | SD | Mean | SD | Mean | SD | Mean | SD | increase | ratio |
| DMSO | | 63.9 | 0.9 | 33.9 | 1.0 | 10.9 | 0.1 | 10.3 | 0.2 | | 0.2 |
| TLR1 | 0.3125 | 66.4 | 1.5 | 21.6 | 0.5 | 15.4 | 0.8 | 19.4 | 0.4 | 1.9 | 0.4 |
| | 0.625 | 63.2 | 0.5 | 12.3 | 0.1 | 17.3 | 0.3 | 25.0 | 0.7 | 2.4 | 0.7 |
| | 1.25 | 58.1 | 2.1 | 6.9 | 1.0 | 16.9 | 0.7 | 27.0 | 0.8 | 2.6 | 0.9 |
| | 2.5 | 52.1 | 1.9 | 2.9 | 0.1 | 15.2 | 0.2 | 27.9 | 1.9 | 2.7 | 1.2 |
| | 5 | 44.5 | 0.4 | 1.7 | 0.1 | 11.9 | 0.5 | 26.2 | 0.9 | 2.5 | 1.4 |
| | 10 | 34.1 | 2.4 | 1.3 | 0.1 | 9.2 | 0.6 | 20.3 | 1.6 | 2.0 | 1.5 |
| TLR2 | 0.3125 | 62.8 | 1.2 | 13.5 | 0.6 | 16.7 | 0.5 | 23.5 | 0.6 | 2.3 | 0.6 |
| | 0.625 | 56.8 | 2.3 | 6.1 | 0.1 | 17.1 | 0.3 | 26.3 | 2.0 | 2.5 | 0.9 |
| | 1.25 | 49.7 | 4.3 | 3.2 | 0.1 | 16.0 | 0.9 | 24.2 | 3.3 | 2.3 | 1.0 |
| | 2.5 | 50.9 | 2.3 | 2.2 | 0.1 | 16.0 | 1.0 | 26.5 | 1.1 | 2.6 | 1.1 |
| | 5 | 50.9 | 0.7 | 2.1 | 0.1 | 16.2 | 0.2 | 26.5 | 0.7 | 2.6 | 1.1 |
| | 10 | 42.4 | 3.7 | 1.8 | 0.2 | 13.6 | 0.8 | 22.0 | 2.2 | 2.1 | 1.1 |
| M9831/ | 0.3125 | 66.8 | 2.0 | 25.0 | 0.7 | 15.8 | 0.8 | 15.8 | 0.2 | 1.5 | 0.3 |
| VX-984 | 0.625 | 66.8 | 1.1 | 16.7 | 0.2 | 17.8 | 0.5 | 22.5 | 0.8 | 2.2 | 0.5 |
| | 1.25 | 60.4 | 1.1 | 9.0 | 0.4 | 18.1 | 0.8 | 25.3 | 0.8 | 2.4 | 0.7 |
| | 2.5 | 59.0 | 1.0 | 4.2 | 0.2 | 18.8 | 0.5 | 28.6 | 0.6 | 2.8 | 0.9 |
| | 5 | 55.0 | 2.3 | 2.4 | 0.1 | 17.1 | 0.8 | 29.0 | 1.8 | 2.8 | 1.1 |
| | 10 | 53.4 | 2.4 | 2.0 | 0.1 | 17.3 | 0.8 | 27.7 | 1.7 | 2.7 | 1.1 |
| AZD7648 | 0.3125 | 69.5 | 0.6 | 28.6 | 0.1 | 15.2 | 0.6 | 15.0 | 0.2 | 1.4 | 0.3 |
| | 0.625 | 66.0 | 1.1 | 18.3 | 0.9 | 17.4 | 0.1 | 20.7 | 0.3 | 2.0 | 0.5 |
| | 1.25 | 53.3 | 12.9 | 8.7 | 1.5 | 16.5 | 3.3 | 20.6 | 6.9 | 2.0 | 0.6 |
| | 2.5 | 60.2 | 1.2 | 3.9 | 0.0 | 19.3 | 0.6 | 29.6 | 0.8 | 2.9 | 1.0 |
| | 5 | 57.5 | 0.3 | 2.5 | 0.1 | 18.5 | 0.5 | 30.2 | 0.1 | 2.9 | 1.1 |
| | 10 | 53.7 | 1.8 | 2.2 | 0.1 | 16.9 | 0.4 | 28.6 | 1.1 | 2.8 | 1.1 |

### Example 9—Effect of PolQ Inhibitors PolQ2 and PolQ6 on Precise Gene Editing Outcome at Various Target-Sites

To assess if PolQ2 and PolQ6 increase precise gene editing at different genomic loci, the inhibitors were tested with different sgRNAs using conditions specified in the experiment below.

HEK293T cells were seeded into 96-well plates and allowed to attach for 20 hours. Two hours before transfections cells were submitted to inhibitor treatments, including the following conditions: a) DMSO control b) 1 μM DNAPK inhibitor AZD7648 c) 1 μM DNAPK inhibitor AZD7648 in combination with 3 μM PolQ inhibitor (PolQ2) d) 1 μM DNAPK inhibitor AZD7648 in combination with 3 μM PolQ inhibitor (PolQ6). Cells were transfected with DNA plasmids encoding for SpCas9-EGFP and a sgRNAs targeting CD34 (gMEJ, gINS) and STAT1 (gDel) presence of single-stranded oligonucleotide donor (ssDNA). 70 hours post-transfection cell confluence and EGFP-based transfection efficiencies were determined with the Incucyte S3. Genomic DNA was extracted and editing outcome was analysed through deep-targeted amplicon sequencing using bioinformatic analysis.

As illustrated in table 3 below both PolQ inhibitors, PolQ2 and PolQ6, increase precise knock-in frequencies of the provided single-stranded oligonucleotide donor in DNAPK inhibited cells across all tested target-sites. Moreover, the tested inhibitor combinations decrease unprecise DNA repair events.

TABLE 3

| | | | | | | | | | | | | Precise | |
|---|---|---|---|---|---|---|---|---|---|---|---|---|---|
| | | DNAPK | PolQ | | | | | | | | | KI | KI to |
| Target- | | inhibitor | Inhibitor | Total editing | | NHEJ indels | | MMEJ indels | | Precise KI | | fold | indel |
| site | Treatment | (μM) | (μM) | Mean | SD | Mean | SD | Mean | SD | Mean | SD | increase | ratio |
| gDel | DMSO | 0 | 0 | 86.2 | 1.9 | 26.6 | 0.8 | 12.4 | 0.4 | 24.4 | 0.8 | — | 0.4 |
| | AZD7648 | 1 | 0 | 77.3 | 2.1 | 1.3 | 0.2 | 8.9 | 1.1 | 61.8 | 1.2 | 2.5 | 4.0 |
| | AZD7648 + | 1 | 3 | 74.0 | 3.2 | 0.8 | 0.0 | 1.9 | 0.5 | 69.7 | 4.0 | 2.9 | 16.3 |
| | PolQ2 | | | | | | | | | | | | |
| | AZD7648 + | 1 | 3 | 71.3 | 2.2 | 0.7 | 0.0 | 0.3 | 0.1 | 70.2 | 2.3 | 2.9 | 63.9 |
| | PolQ6 | | | | | | | | | | | | |
| glns | DMSO | 0 | 0 | 87.4 | 1.4 | 48.0 | 1.7 | 10.5 | 0.4 | 19.3 | 1.1 | — | 0.3 |
| | AZD7648 | 1 | 0 | 83.0 | 0.3 | 1.3 | 0.2 | 13.3 | 0.8 | 63.6 | 1.0 | 3.3 | 3.3 |
| | AZD7648 + | 1 | 3 | 79.5 | 1.7 | 1.1 | 0.0 | 2.1 | 0.4 | 75.5 | 1.9 | 3.9 | 19.3 |
| | PolQ2 | | | | | | | | | | | | |
| | AZD7648 + | 1 | 3 | 77.5 | 2.1 | 0.9 | 0.1 | 0.8 | 0.1 | 75.7 | 2.2 | 3.9 | 40.8 |
| | PolQ6 | | | | | | | | | | | | |
| gMej | DMSO | 0 | 0 | 89.6 | 1.0 | 18.5 | 0.2 | 24.4 | 0.4 | 19.5 | 1.0 | — | 0.3 |
| | AZD7648 | 1 | 0 | 83.1 | 0.8 | 2.9 | 3.1 | 27.9 | 1.9 | 42.0 | 9.2 | 2.2 | 1.0 |
| | AZD7648 + | 1 | 3 | 79.4 | 3.6 | 0.4 | 0.0 | 3.6 | 0.3 | 73.2 | 3.3 | 3.8 | 11.8 |
| | PolQ2 | | | | | | | | | | | | |
| | AZD7648 + | 1 | 3 | 79.0 | 3.5 | 0.3 | 0.0 | 1.4 | 0.3 | 76.8 | 3.8 | 3.9 | 34.9 |
| | PolQ6 | | | | | | | | | | | | |

### Example 10—Effect of PolQ Inhibitor ART558 on Precise Gene Editing Outcomes

To test the potency of the PolQ inhibitor ART558 for precise gene editing, the inhibitor was titrated using conditions specified in the experiment below.

HEK293T cells were seeded into 96-well plates and allowed to attach for 20 hours. Two hours before transfections cells were submitted to inhibitor treatments, including the following conditions: a) 1 μM DNAPK inhibitor AZD7648 b) 1 μM DNAPK inhibitor AZD7648 in combination with 0.1, 0.3, 1, 3 10 μM PolQ inhibitor (ART558). Cells were transfected with DNA plasmids encoding for SpCas9-EGFP and a sgRNAs targeting CD34 (gMEJ) presence of single-stranded oligonucleotide donor (ssDNA). 70 hours post-transfection cell confluence and EGFP-based transfection efficiencies were determined with the Incucyte S3. Genomic DNA was extracted and editing outcome was analysed through deep-targeted amplicon sequencing using Crispresso2 bioinformatic analysis. As illustrated in table 4 below, ART558 increases precise knock-in frequencies of the provided single-stranded oligonucleotide donor in a concentration-dependent manner and decreases unprecise DNA repair events with increasing inhibitor concentration.

TABLE 4

| | | | | | | | | | | | Precise | |
|---|---|---|---|---|---|---|---|---|---|---|---|---|
| | DNAPK | PolQ | | | | | | | | | KI | KI to |
| | inhibitor | Inhibitor | Total editing | | NHEJ indels | | MMEJ indels | | Precise KI | | fold | indel |
| Treatment | (μM) | (μM) | Mean | SD | Mean | SD | Mean | SD | Mean | SD | increase | ratio |
| AZD7648 | 1 | 0 | 74.4 | 1.4 | 1.3 | 0.0 | 32.6 | 0.2 | 31.7 | 1.2 | — | 0.7 |
| AZD7648 + | 1 | 0.1 | 72.5 | 1.1 | 1.5 | 0.1 | 27.1 | 0.3 | 34.1 | 1.1 | 1.1 | 0.9 |
| ART558 | 1 | 0.3 | 68.7 | 2.1 | 1.5 | 0.1 | 20.2 | 0.5 | 38.8 | 1.6 | 1.2 | 1.3 |
| | 1 | 1 | 65.3 | 0.5 | 1.5 | 0.0 | 12.6 | 0.6 | 46.2 | 0.7 | 1.5 | 2.4 |
| | 1 | 3 | 64.8 | 5.4 | 1.7 | 0.3 | 5.7 | 0.5 | 55.4 | 5.7 | 1.7 | 5.9 |
| | 1 | 10 | 60.0 | 5.8 | 1.5 | 0.0 | 6.2 | 5.9 | 50.6 | 2.2 | 1.6 | 5.4 |

### Example 11—Combinatorial Treatment with PolQ Inhibitors Targeting Both Functional Enzyme Domains

To maintain genome integrity upon DNA double-strand breaks cells developed different mechanisms to repair broken DNA ends. Besides non-homologous end-joining (NHEJ) and homologous recombination (HR), cells evolved the error-prone microhomology-mediated end-joining (MMEJ) DNA repair pathway. DNA polymerase theta (PolQ) is a key enzyme mediating MMEJ repair. PolQ a multidomain enzyme comprises a N-terminal helicase-like function, an unstructured central domain, and a C-terminal polymerase domain. Both functional protein units are involved in PolQ-mediated DNA repair and can be inhibited using domain-specific inhibitors. The experiment addresses the question if simultaneous inhibition of both functional PolQ domains enhances the effect on gene editing outcome, compared to targeting of individual domains.

HEK293T cells were seeded into 96-well plates and allowed to attach for 20 hours. Two hours before transfections cells were submitted to inhibitor treatments, including the following conditions: a) DMSO control, b) 1 μM DNAPK inhibitor AZD7648 in combination with 1 and 2 μM polymerase-domain-targeting PolQ inhibitor (PolQ2), c) 1 μM DNAPK inhibitor AZD7648 in combination with 1 and 2 μM helicase-domain-targeting PolQ inhibitor (PolQ6) and d) 1 μM DNAPK inhibitor AZD7648 in combination with 0.5 μM polymerase- and helicase-domain-targeting PolQ inhibitor (PolQ2 & PolQ6) and 1 μM polymerase- and helicase-domain-targeting PolQ inhibitor (PolQ2 & PolQ6). Cells were transfected with DNA plasmids encoding for SpCas9-EGFP and a sgRNA targeting CD34 (gMEJ) together with a single-stranded oligonucleotide donor (ssDNA). 70 hours post-transfection cell confluence and EGFP-based transfection efficiencies were determined with the Incucyte S3. Genomic DNA was extracted and editing outcome was analysed through deep-targeted amplicon sequencing using RIMA for KI bioinformatic analysis.

As illustrated by the data shown in table 5, Combined PolQ inhibitor treatments, targeting both functional PolQ domains, exhibit a larger increase on targeted knock-in and concomitant decrease of unprecise DNA repair products when compared to individual PolQ inhibitors only targeting one functional enzyme domain at the same concentration.

TABLE 5

| | PolQ | | | | | | | | |
|---|---|---|---|---|---|---|---|---|---|
| | Inhibitor | Total editing | | NHEJ indels | | MMEJ indels | | Precise KI | |
| Treatment | (μM) | Mean | SD | Mean | SD | Mean | SD | Mean | SD |
| DMSO | 0 | 74.78 | 2.47 | 1.3 | 0.02 | 33.6 | 0.38 | 31.0 | 2.05 |
| PolQ2 | 1 | 65.9 | 1.44 | 1.6 | 0.09 | 11.0 | 0.67 | 48.7 | 0.61 |
| PolQ6 | 1 | 63.4 | 5.77 | 1.3 | 0.15 | 3.8 | 0.48 | 58.1 | 6.08 |
| PolQ2 | 2 | 65.3 | 0.75 | 1.6 | 0.04 | 5.6 | 0.24 | 56.2 | 1.05 |
| PolQ6 | 2 | 66.9 | 0.78 | 1.5 | 0.04 | 2.0 | 0.14 | 63.8 | 0.79 |
| PolQ2 & | 0.5 | 66.8 | 0.66 | 1.5 | 0.07 | 1.4 | 0.09 | 64.4 | 0.68 |
| PolQ6 | | | | | | | | | |
| PolQ2 & | 1 | 65.5 | 1.65 | 1.5 | 0.07 | 0.8 | 0.09 | 63.9 | 1.61 |
| PolQ6 | | | | | | | | | |

### Example 12—Evaluation of Off-Target Editing in the Presence of DNAPK and PolQ Inhibitors

To test the effect of the DNAPK/PolQ inhibitor combination on off-target editing established HEK3 and HEK4 on- and off-target sites were analysed in the experiment below.

HEK293T cells were seeded into 96-well plates and allowed to attach for 20 hours. Two hours before transfections cells were submitted to inhibitor treatments, including the following conditions: a) DMSO control, b) 1 μM DNAPK inhibitor AZD7648 c) 1 μM DNAPK inhibitor AZD7648 in combination with 3 μM polymerase-domain-targeting PolQ inhibitor (PolQ2), and d) 1 μM DNAPK inhibitor AZD7648 in combination with 3 μM helicase-domain-targeting PolQ inhibitior (PolQ6). Cells were transfected with DNA plasmids encoding for SpCas9-EGFP and a sgRNAs targeting established HEK3 and HEK4 off-target sites in the absence and presence of single-stranded oligonucleotide donor (ssDNA). 70 hours post-transfection cell confluence and EGFP-based transfection efficiencies were determined with the Incucyte S3. Genomic DNA was extracted and editing outcome was analysed through deep-targeted amplicon sequencing using Crispresso2 bioinformatic analysis.

As shown in the table 6 below the reduction of on- and off-target editing with DNAPK inhibitor, the effect is even more pronounced when DNAPK inhibitor is combined with PolQ inhibitors. The presence of single-stranded oligonucleotide donor reduces on- and off-target editing by about 20% in comparison to no DNA donor samples. The reduction of on-target editing in the presence of DNAPK and PolQ inhibitor is partially restored in the presence of single-stranded oligonucleotide donor, while off-targets are reduced.

TABLE 6

| Target- | | | | | | | | | | | | | |
|---|---|---|---|---|---|---|---|---|---|---|---|---|---|
| site & | | DNAPK | PolQ | | | | | | | | | | |
| DNA | | inhibitor | Inhibitor | on-target | | OT1 | | OT2 | | OT3 | | OT4 | |
| donor | Treatment | (μM) | (μM) | Mean | SD | Mean | SD | Mean | SD | Mean | SD | Mean | SD |
| HEK3 | CTR | 0 | 0 | 0.1 | 0.0 | 0.0 | 0.0 | 0.0 | 0.0 | 0.0 | 0.0 | 0.0 | 0.0 |
| nodonor | DMSO | 0 | 0 | 89.3 | 2.2 | 42.3 | 0.9 | 44.4 | 2.4 | 17.2 | 1.5 | 0.5 | 0.1 |
| | AZD7648 | 1 | 0 | 73.8 | 3.0 | 41.9 | 1.5 | 32.5 | 1.2 | 16.3 | 0.6 | 0.3 | 0.0 |
| | AZD7648 + | 1 | 3 | 34.3 | 0.6 | 22.4 | 0.4 | 15.0 | 0.5 | 5.8 | 0.4 | 0.1 | 0.0 |
| | PolQ2 | | | | | | | | | | | | |
| | AZD7648 + | 1 | 3 | 24.0 | 3.5 | 8.3 | 1.1 | 3.8 | 1.2 | 1.9 | 0.5 | 0.1 | 0.0 |
| | PolQ6 | | | | | | | | | | | | |
| HEK3 | DMSO | 0 | 0 | 72.5 | 7.1 | 20.8 | 3.3 | 23.6 | 4.5 | 7.9 | 1.7 | 0.3 | 0.0 |
| ssDNA | AZD7648 | 1 | 0 | 61.5 | 2.7 | 17.7 | 1.1 | 12.7 | 0.6 | 7.6 | 0.6 | 0.1 | 0.0 |
| | AZD7648 + | 1 | 3 | 54.7 | 3.1 | 7.3 | 0.8 | 5.1 | 0.5 | 2.0 | 0.3 | 0.1 | 0.0 |
| | PolQ2 | | | | | | | | | | | | |
| | AZD7648 + | 1 | 3 | 57.0 | 2.9 | 3.5 | 0.6 | 1.4 | 0.2 | 0.8 | 0.1 | 0.0 | 0.0 |
| | PolQ6 | | | | | | | | | | | | |

| Target- | | | | | | | | | | | |
|---|---|---|---|---|---|---|---|---|---|---|---|
| site & | | DNAPK | PolQ | | | | | | | | |
| DNA | | inhibitor | Inhibitor | on-target | | OT1 | | OT2 | | OT3 | |
| donor | Treatment | (μM) | (μM) | Mean | SD | Mean | SD | Mean | SD | Mean | SD |
| HEK4 | CTR | 0 | 0 | 0.1 | 0.0 | 0.0 | 0.0 | 0.1 | 0.0 | 0.0 | 0.0 |
| nodonor | DMSO | 0 | 0 | 67.2 | 4.7 | 44.4 | 1.8 | 44.5 | 2.2 | 40.4 | 1.4 |
| | AZD7648 | 1 | 0 | 9.8 | 1.9 | 12.7 | 2.1 | 7.3 | 1.8 | 36.3 | 4.8 |
| | AZD7648 + | 1 | 3 | 1.5 | 0.0 | 1.0 | 0.3 | 0.5 | 0.1 | 4.6 | 0.6 |
| | PolQ2 | | | | | | | | | | |
| | AZD7648 + | 1 | 3 | 1.1 | 0.4 | 0.6 | 0.1 | 0.6 | 0.2 | 0.8 | 0.1 |
| | PolQ6 | | | | | | | | | | |
| HEK4 | DMSO | 0 | 0 | 48.4 | 2.0 | 24.0 | 0.7 | 27.6 | 0.9 | 23.5 | 1.1 |
| ssDNA | AZD7648 | 1 | 0 | 15.6 | 2.1 | 5.4 | 1.1 | 3.3 | 0.7 | 18.2 | 2.5 |
| | AZD7648 + | 1 | 3 | 13.2 | 1.6 | 0.4 | 0.2 | 0.3 | 0.0 | 1.3 | 0.5 |
| | PolQ2 | | | | | | | | | | |
| | AZD7648 + | 1 | 3 | 14.5 | 1.2 | 0.4 | 0.0 | 0.5 | 0.2 | 0.6 | 0.1 |
| | PolQ6 | | | | | | | | | | |

What is claimed:

1. A method of inserting a polynucleotide of interest into a genome of a eukaryotic cell, the method comprising:
a. adding an inhibitor of the microhomology-mediated end joining (MMEJ) pathway to a composition comprising the eukaryotic cell,
b. adding a Cas effector protein to the composition,
c. adding the polynucleotide of interest to the composition,
wherein the polynucleotide of interest is inserted into the genome by homology directed repair (HDR) or single-stranded template repair (SSTR).

2. The method of claim 1, wherein (a) further comprises adding an inhibitor of the non-homologous end joining (NHEJ) pathway.

3. The method of claim 1 or 2, further comprising: (d) adding a polynucleotide comprising: an RNA guide sequence; a Cas-binding region; a DNA template sequence, or combinations thereof to the composition.

4. The method of any of claims 1-3, wherein the Cas effector protein is added in (b) by adding a Cas polynucleotide encoding the Cas effector protein.

5. The method of any of claims 1-4, wherein one or more of (i) the polynucleotide of interest, (ii) the polynucleotide of (d), or (iii) the Cas polynucleotide are encoded on a vector.

6. The method of any of claims 1-4, wherein (i) the polynucleotide of interest, (ii) the polynucleotide of step (d), and (iii) the Cas polynucleotide are encoded on a single vector.

7. The method of any of claims 1-6, wherein the polynucleotide of interest is added as DNA.

8. The method of any of claims 1-6, wherein the polynucleotide of step (d) is added as DNA.

9. The method any of claims 1-6, wherein the polynucleotide of step (d) is added as RNA.

10. The method of any of claims 1-6, wherein the Cas effector polynucleotide is added as DNA.

11. The method any of claims 1-6, wherein the Cas polynucleotide is added as RNA.

12. The method of any of claims 1-6, wherein the Cas polynucleotide is added as mRNA.

13. The method of claim 5 or 6, wherein the vector is a viral vector.

14. The method of claim 13, wherein the viral vector is a retrovirus, a lentivirus, an adenovirus, or an adeno-associated virus (AAV).

15. The method of claim 3, wherein the Cas effector protein and the polynucleotide of (d) are added in the form of a ribonucleoprotein (RNP).

16. The method of any of claims 1-15, wherein the Cas effector protein, the polynucleotide of interest, and the polynucleotide of (d) are added to the cell by microinjection, electroporation, or via a lipid nanoparticle, liposome, exosome, gold nanoparticle, or a DNA nanoclew.

17. The method of claim 5 or 9, wherein the vector is added to the composition by transfecting the eukaryotic cell.

18. The method of any of claims 1-17, wherein the Cas effector protein is a Cas9 nuclease, a Cas12a nuclease, or a Cas12f nuclease.

19. The method of claim 18, wherein the Cas effector protein is a Cas9 nuclease.

20. The method of claim 19, wherein the Cas9 nuclease is a Cas9 nuclease fused to a reverse transcriptase, a Cas9 nuclease fused to a DNA polymerase, a Cas9 nuclease fused to DN1S, a Cas9 nickase, a Cas9 fused to a Geminin degron domain, or a Cas9 nuclease fused to CTIP.

21. The method of any of claims 1-20, wherein the polynucleotide of interest is added via a vector.

22. The method of claim 21, wherein the vector is a viral vector.

23. The method of claim 22, wherein the viral vector is a retrovirus, a lentivirus, an adenovirus, or an adeno-associated virus (AAV).

24. The method of any of claims 1-23, wherein the polynucleotide of interest comprises a gene of interest.

25. The method of any of claims 1-23, wherein the polynucleotide of interest is 1 to 50 base pairs in length.

26. The method of any of claims 1-23, wherein the polynucleotide of interest is 50 to 5000 base pairs in length.

27. The method of any of claims 1-23, wherein the polynucleotide of interest is single stranded.

28. The method of any of claims 1-23, wherein the polynucleotide of interest is double stranded.

29. The method of any of claims 1-23, wherein the polynucleotide of interest is a hybrid polynucleotide comprising single-stranded and double-stranded regions.

30. The method of claim 29, wherein the hybrid polynucleotide comprises double-stranded sequences at the 5′ and 3′ ends and an internal single-stranded sequence.

31. The method of any of claims 1-28, wherein the polynucleotide of interest is double stranded with blunt ends.

32. The method of any of claims 1-30, wherein the polynucleotide of interest is double stranded with a 3′ overhang.

33. The method of any of claims 1-30, wherein the polynucleotide of interest is double stranded with a 5′ overhang.

34. The method of any of claims 1-29, wherein the polynucleotide of interest is a circular polynucleotide.

35. The method of any of claims 1-34, wherein the polynucleotide of interest comprises a chemical modification which enhances the stability, activity, distribution, or uptake of the polynucleotide.

36. The method of any of claims 1-35, wherein the inhibitor of the MMEJ pathway is an inhibitor of PolQ.

37. The method of claim 36, wherein the inhibitor of PolQ is PolQ_1, PolQ_2, PolQ_3, PolQ_4, PolQ_5, PolQ_6, PolQ_7, or combinations thereof.

38. The method of claim 36, wherein the inhibitor of PolQ is a peptide.

39. The method of any of claims 1-38, wherein the concentration of the inhibitor of the MMEJ pathway in the composition is about 0.01 μM to about 1 mM.

40. The method of any of claims 1-38, wherein the concentration of the inhibitor of the MMEJ pathway in the composition is about 0.1 μM to about 100 μM.

41. The method of any of claims 2-38, wherein the inhibitor of the NHEJ pathway is an inhibitor of DNA-dependent protein kinase (DNA-PK).

42. The method of claim 41, wherein the inhibitor of DNA-PK is M3814, M9831/VX984, Nu7441, Nu7026, KU0060648, AZD7648, or combinations thereof.

43. The method of claim 42, wherein the inhibitor of DNA-PK is AZD7648.

44. The method of claim 41, wherein the inhibitor of DNA-PK is a peptide.

45. The method of any of claims 2-44, wherein the concentration of the inhibitor of the NHEJ pathway in the composition is about 0.01 μM to about 1 mM.

46. The method of any of claims 2-44, wherein the concentration of the inhibitor of the NHEJ pathway in the composition is about 0.1 μM to about 100 μM.

47. The method of any of claims 1-46, wherein the inhibitor of the MMEJ pathway is added to the composition 0 minutes to about 48 hours before the Cas effector protein is added to the composition.

48. The method of any of claims 1-46, wherein the inhibitor of the MMEJ pathway is added to the composition 0 minutes to about 24 hours before the Cas effector protein is added to the composition.

49. The method of any of claims 1-46, wherein the inhibitor of the MMEJ pathway is added to the composition 0 minutes to about 6 hours before the Cas effector protein is added to the composition.

50. The method of any of claims 1-46, wherein the inhibitor of the MMEJ pathway is added to the composition 0 minutes to about 1 hour after the Cas effector protein is added to the composition.

51. The method of any of claims 2-50, wherein the inhibitor of the NHEJ pathway is added to the composition 0 minutes to about 48 hours before the Cas effector protein is added to the composition.

52. The method of any of claims 2-50, wherein the inhibitor of the NHEJ pathway is added to the composition 0 minutes to about 24 hours before the Cas effector protein is added to the composition.

53. The method of any of claims 2-50, wherein the inhibitor of the NHEJ pathway is added to the composition 0 minutes to about 6 hours before the Cas effector protein is added to the composition.

54. The method of any of claims 2-50, wherein the inhibitor of the NHEJ pathway is added to the composition 0 minutes to about 1 hour after the Cas effector protein is added to the composition.

55. The method of any of claims 2-54, wherein the inhibitor of the MMEJ pathway and the inhibitor of the NHEJ pathway are added to the composition at the same time.

56. The method of any of claims 2-54, wherein the inhibitor of the MMEJ pathway and the inhibitor of the NHEJ pathway are added to the composition at different times.

57. The method of any of claims 2-54, wherein the inhibitor of the MMEJ pathway, the inhibitor of the NHEJ pathway, and the Cas effector protein as added to the composition at the same time.

58. The method of any of claims 1-57, wherein the inhibitor of the MMEJ pathway is in the composition for about 1 to about 300 hours.

59. The method of any of claims 1-57, wherein the inhibitor of the MMEJ pathway is in the composition for about 10 to about 100 hours.

60. The method of any of claims 1-57, wherein the inhibitor of the MMEJ pathway is added at least once, at least twice, or at least three times.

61. The method of any of claims 2-60, wherein the inhibitor of the NHEJ pathway is in the composition for about 1 to about 300 hours.

62. The method of any of claims 2-60, wherein the inhibitor of the NHEJ pathway is in the composition for about 10 to about 100 hours.

63. The method of any of claims 2-60, wherein the inhibitor of the NHEJ pathway is added at least once, at least twice, or at least three times.

64. The method of any of claims 1-63 wherein the composition comprising the eukaryotic cells is a cell culture.

65. The method of claim 64, wherein the cell culture is an in vitro cell culture or an ex vivo cell culture.

66. The method of any of claims 1-65, wherein the eukaryotic cell is in vivo.

67. The method of claim 64, wherein the cell culture comprises a cell extract.

68. The method of any of claims 1-67, wherein the eukaryotic cell is a lymphocyte.

69. The method of claim 68, wherein the lymphocyte comprises a chimeric antigen receptor (CAR) or a T cell receptor (TCR).

70. The method of any of claims 1-67, wherein the eukaryotic cell is a pluripotent stem cell.

71. The method of claim 70, wherein the pluripotent stem cell is an induced pluripotent stem cell.

72. The method of claim 64, wherein the cell culture is a mammalian cell culture.

73. A method of inserting a polynucleotide of interest into a genome of a eukaryotic cell, the method comprising:
a. adding an inhibitor of the microhomology-mediated end joining (MMEJ) pathway to a composition comprising the eukaryotic cell,
b. adding the polynucleotide of interest to the composition,
wherein the genome comprises a genomically integrated Cas polynucleotide, and
wherein the polynucleotide of interest is inserted into the genome by homology directed repair (HDR) or single-stranded template repair (SSTR).

74. The method of claim 73, wherein (a) further comprises adding an inhibitor of the non-homologous end joining (NHEJ) pathway to the composition.

75. The method of claim 73 or 74, further comprising: (c) adding a polynucleotide comprising: an RNA guide sequence; a Cas-binding region; a DNA template sequence, or combinations thereof to the composition.

76. The method of claim 75, wherein (i) the polynucleotide of interest and (ii) the polynucleotide of (c) are encoded on a vector.

77. The method of any of claims 73-76, wherein the polynucleotide of interest is added as DNA.

78. The method of any of claims 75-77, wherein the polynucleotide of (c) is added as DNA.

79. The method of any of claims 75-77, wherein the polynucleotide of (c) is added as RNA.

80. The method of claim 76, wherein the vector is a viral vector.

81. The method of claim 80, wherein the viral vector is a retrovirus, a lentivirus, an adenovirus, or an adeno-associated virus (AAV).

82. The method of claim 76, wherein the vector is added to the composition by transfecting the eukaryotic cell.

83. The method of any of claims 73-82, wherein the genomically integrated Cas polynucleotide is inducible.

84. The method of any of claims 73-83, wherein the Cas effector protein is a Cas9 nuclease, a Cas12a nuclease, or a Cas12f nuclease.

85. The method of claim 84, wherein the Cas effector protein is a Cas9 nuclease.

86. The method of claim 85, wherein the Cas9 nuclease is a Cas9 nuclease fused to a reverse transcriptase, a Cas9 nuclease fused to a DNA polymerase, a Cas9 nuclease fused to DN1S, a Cas9 nickase, a Cas9 fused to a Geminin degron domain or a Cas9 nuclease fused to CTIP.

87. The method of any of claims 73-86, wherein the polynucleotide of interest is added via a vector.

88. The method of claim 87, wherein the vector is a viral vector.

89. The method of claim 88, wherein the viral vector is a retrovirus, a lentivirus, an adenovirus, or an adeno-associated virus (AAV).

90. The method of any of claims 73-89, wherein the polynucleotide of interest comprises a gene of interest.

91. The method of any of claims 73-90, wherein the polynucleotide of interest is 1 to 50 base pairs in length.

92. The method of any of claims 73-90, wherein the polynucleotide of interest is 50 to 5000 base pairs in length.

93. The method of any of claims 73-90, wherein the polynucleotide of interest is single stranded.

94. The method of any of claims 73-90, wherein the polynucleotide of interest is double stranded.

95. The method of any of claims 73-90, wherein the polynucleotide of interest is a hybrid polynucleotide comprising single-stranded and double-stranded regions.

96. The method of claim 95, wherein the hybrid polynucleotide comprises double-stranded sequences at the 5′ and 3′ ends and an internal single-stranded sequence

97. The method of any of claims 73-90, wherein the polynucleotide of interest is double stranded with blunt ends.

98. The method of any of claims 73-90, wherein the polynucleotide of interest is double stranded with a 3′ overhang.

99. The method of any of claims 73-90, wherein the polynucleotide of interest is double stranded with a 5′ overhang.

100. The method of any of claims 73-90, wherein the polynucleotide is a circular polynucleotide.

101. The method of any of claims 73-100, wherein the polynucleotide comprises a chemical modification which enhances the stability, activity, distribution, or uptake of the polynucleotide.

102. The method of any of claims 73-101, wherein the inhibitor of the MMEJ pathway is an inhibitor of PolQ.

103. The method of claim 102, wherein the inhibitor of PolQ is PolQ_1, PolQ_2, PolQ_3, PolQ_4, PolQ_5, PolQ_6, PolQ_7, or combinations thereof.

104. The method of claim 102, wherein the inhibitor of PolQ is a peptide.

105. The method of any of claims 73-104, wherein the concentration of the inhibitor of the MMEJ pathway in the composition is about 0.01 μM to about 1 mM.

106. The method of any of claims 73-104, wherein the concentration of the inhibitor of the MMEJ pathway in the composition is about 0.1 μM to about 100 μM.

107. The method of any of claims 74-106, wherein the inhibitor of the NHEJ pathway is an inhibitor of DNA-dependent protein kinase (DNA-PK).

108. The method of claim 107, wherein the inhibitor of DNA-PK is M3814, M9831/VX984, Nu7441, Nu7026, KU0060648, AZD7648, or combinations thereof.

109. The method of claim 107, wherein the inhibitor of DNA-PK is a peptide.

110. The method of claim 109, wherein the inhibitor of DNA-PK is AZD7648.

111. The method of any of claims 74-110, wherein the concentration of the inhibitor of the NHEJ pathway in the composition is about 0.01 μM to about 1 mM.

112. The method of any of claims 74-110, wherein the concentration of the inhibitor of the NHEJ pathway in the composition is about 0.1 μM to about 100 μM.

113. The method of any of claims 73-112, wherein the inhibitor of the MMEJ pathway is added to the composition 0 minutes to about 48 hours before induction of the genomically integrated Cas polynucleotide.

114. The method of any of claims 73-112, wherein the inhibitor of the MMEJ pathway is added to the composition 0 minutes to about 24 hours before induction of the genomically integrated Cas polynucleotide.

115. The method of any of claims 73-112, wherein the inhibitor of the MMEJ pathway is added to the composition 0 minutes to about 6 hours before induction of the genomically integrated Cas polynucleotide.

116. The method of any of claims 73-115, wherein the inhibitor of the NHEJ pathway is added to the composition 0 minutes to about 24 hours before the induction of the genomically integrated Cas polynucleotide.

117. The method of any of claims 74-115, wherein the inhibitor of the NHEJ pathway is added to the composition 0 minutes to about 24 hours before the induction of the genomically integrated Cas polynucleotide.

118. The method of any of claims 74-115, wherein the inhibitor of the NHEJ pathway is added to the composition 0 minutes to about 6 hours before induction of the genomically integrated Cas polynucleotide.

119. The method of any of claims 74-118, wherein the inhibitor of the MMEJ pathway and the inhibitor of the NHEJ pathway are added to the composition at the same time.

120. The method of any of claims 74-118, wherein the inhibitor of the MMEJ pathway and the inhibitor of the NHEJ pathway are added to the composition at different times.

121. The method of any of claims 74-120, wherein the inhibitor of the MMEJ pathway and the inhibitor of the NHEJ pathway are added to the composition at the same time as induction of the genomically integrated Cas polynucleotide.

122. The method of any of claims 73-121, wherein the inhibitor of the MMEJ pathway is in the composition for about 1 to about 300 hours.

123. The method of any of claims 73-121, wherein the inhibitor of the MMEJ pathway is in the composition for about 10 to about 100 hours.

124. The method of any of claims 73-123, wherein the inhibitor of the MMEJ pathway is added at least once, at least twice, or at least three times.

125. The method of any of claims 74-124, wherein the inhibitor of the NHEJ pathway is in the composition for about 1 to about 300 hours.

126. The method of any of claims 74-124, wherein the inhibitor of the NHEJ pathway is in the composition for about 10 to about 100 hours.

127. The method of any of claims 74-126, wherein the inhibitor of the NHEJ pathway is added at least once, at least twice, or at least three times.

128. The method of any of claims 73-127 wherein the composition comprising the eukaryotic cells is a cell culture.

129. The method of claim 128, wherein the cell culture is an in vitro cell culture or an ex vivo cell culture.

130. The method of any of claims 73-129, wherein the eukaryotic cell is in vivo.

131. The method of claim 130, wherein the cell culture comprises a cell extract.

132. The method of any of claims 73-131, wherein the eukaryotic cell is a lymphocyte.

133. The method of claim 132, wherein the lymphocyte comprises a chimeric antigen receptor or a T Cell receptor (TCR).

134. The method of any of claims 73-131, wherein the eukaryotic cell is a pluripotent stem cell.

135. The method of claim 134, wherein the pluripotent stem cell is an induced pluripotent stem cell.

136. The method of claim 131, wherein the cell culture is a mammalian cell culture.

137. A method of inserting a polynucleotide into a genome of a eukaryotic cell, the method comprising:
a. adding an inhibitor of the microhomology-mediated end joining (MMEJ) pathway to a composition comprising the eukaryotic cell,
b. transfecting the eukaryotic cell with:
i. a vector encoding a Cas effector protein,
ii. a vector comprising a polynucleotide of interest,
iii. a vector comprising a polynucleotide comprising: an RNA guide sequence; a Cas-binding region; a DNA template sequence, or combinations thereof,
wherein the vector of (i), (ii) and (iii) can be on the same vector or different vectors, and wherein the polynucleotide of interest is inserted into the genome by homology directed repair (HDR) or single-stranded template repair (SSTR).

138. The method of claim 137, further comprising adding an inhibitor of the non-homologous end joining (NHEJ) pathway to the composition comprising the eukaryotic cell.

139. The method of claim 137 or 138, wherein the Cas effector protein is encoded by a Cas polynucleotide.

140. The method of any of claims 137-139, wherein (i) the Cas effector protein and (ii) the polynucleotide of interest are encoded on a vector.

141. The method of any of claims 137-139, wherein the Cas effector protein and the polynucleotide of (iii) are encoded on a vector.

142. The method of any of claims 137-139, wherein the Cas effector protein, the polynucleotide of interest, and the polynucleotide of (iii) are encoded on a single vector.

143. The method of claim 137 or 138, wherein the Cas effector protein and the polynucleotide of (iii) are added in the form of a ribonucleoprotein (RNP).

144. A method of increasing the efficiency of homology directed repair (HDR) and single-stranded template repair (SSTR) gene insertions in a eukaryotic cell, the method comprising adding an inhibitor of the microhomology-mediated end joining (MMEJ) pathway when performing CRISPR/Cas mediated gene insertions in the eukaryotic cell.

145. The method of claim 144, further comprising adding an inhibitor of the non-homologous end joining (NHEJ) pathway.

146. The method of claim 144 or 145, wherein the CRISPR/Cas-mediated gene insertion is a CRISPR/Cas9-mediated gene insertion.

147. A method of reducing microhomology-mediated end joining (MMEJ) pathway recombination during CRISPR/Cas mediated gene insertion in a cell, the method comprising adding an inhibitor of the microhomology-mediated end joining (MMEJ) pathway to the cell when performing Cas-mediated gene insertions.

148. The method of claim 147, further comprising reducing non-homologous end joining (NHEJ) recombination during CRISPR/Cas-mediated gene insertion in a cell comprising adding an inhibitor of the non-homologous end joining (NHEJ) pathway to the cell.

149. The method of claim 147 or 148, wherein the CRISPR/Cas-mediated gene insertions are CRISPR/Cas9-mediated gene insertions.

150. A composition comprising:
a. a Cas effector protein or a vector encoding a Cas effector protein; and
b. an inhibitor of the microhomology-mediated end joining (MMEJ) pathway.

151. The composition of claim 150, further comprising an inhibitor of the non-homologous end joining (NHEJ) pathway.

152. The composition of claim 150 or 151, further comprising a polynucleotide comprising: at least one RNA guide sequence; a Cas-binding region; a DNA template sequence, or combinations thereof.

153. The composition of any of claims 150-152, wherein the Cas effector protein is a Cas9 nuclease, a Cas12a nuclease, or a Cas12f nuclease.

154. The method of claim 153, wherein the Cas effector protein is a Cas9 nuclease.

155. The method of claim 154, wherein the Cas9 nuclease is a Cas9 nuclease fused to a reverse transcriptase, a Cas9 fused to a DNA polymerase, a Cas9 fused to DN1S, a Cas9 nickase, a Cas9 fused to a Geminin degron domain, or a Cas9 nuclease fused to CTIP.

156. The composition of any of claims 150-155, wherein the vector encoding a Cas effector protein is a viral vector.

157. The composition of any of claims 150-155, wherein the polynucleotide comprising at least one guide RNA sequence, a Cas-binding region, a DNA template sequence, or combinations thereof, is encoded on a vector.

158. The composition of claim 157, wherein the vector encoding the polynucleotide comprising at least one guide RNA sequence, a Cas-binding region, a DNA template sequence, or combinations thereof, is a viral vector.

159. The composition of claim 150 or 151, wherein the Cas effector protein and the polynucleotide comprising at least one guide RNA sequence, a Cas-binding region, a DNA template sequence, or combinations thereof, are in the form of a ribonucleoprotein (RNP).

160. The composition of any of claims 150-159, further comprising a pharmaceutically acceptable carrier, diluent, or excipient.

161. A kit comprising:
a. a Cas effector protein or a vector encoding a Cas effector protein; and
b. an inhibitor of the microhomology-mediated end joining (MMEJ) pathway.

162. The kit of claim 161, further comprising an inhibitor of the non-homologous end joining (NHEJ) pathway.

163. The kit of claim 161 or 162, further comprising a polynucleotide comprising: at least one RNA guide sequence; a Cas-binding region; a DNA template sequence, or combinations thereof.

164. The kit of any of claims 161-163, wherein the Cas effector protein is a Cas9 nuclease, a Cas12a nuclease, or a Cas12f nuclease.

165. The kit of claim 164, wherein the Cas effector protein is a Cas9 nuclease.

166. The kit of claim 165, wherein the Cas9 nuclease is a Cas9 nuclease fused to a reverse transcriptase, a Cas9 fused to a DNA polymerase, a Cas9 fused to DN1S, a Cas9 nickase, a Cas9 fused to a Geminin degron domain, or a Cas9 nuclease fused to CTIP.

167. The kit of any of claims 161-166, wherein the vector encoding a Cas effector protein is a viral vector.

168. The kit of any of claims 161-167, wherein the guide polynucleotide is encoded on a vector.

169. The kit of claim 168, wherein the vector encoding the guide polynucleotide is a viral vector.

170. The kit of claim 161 or 162, wherein the Cas effector protein and the guide polynucleotide are in the form of a ribonucleoprotein (RNP).


SEQUENCE LISTING (18 sequences)

SEQ ID NO: 1 - protein, 1409 aa, unidentified
  source 1..1409: note = Naturally occuring peptides
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 2 - protein, 1367 aa, unidentified
  source 1..1367: note = Naturally occuring peptides
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1053 aa, unidentified
  source 1..1053: note = Naturally occuring peptides
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 1300 aa, unidentified
  source 1..1300: note = Naturally occuring peptides
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 5 - protein, 1307 aa, unidentified
  source 1..1307: note = Naturally occuring peptides
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 6 - protein, 1129 aa, unidentified
  source 1..1129: note = Naturally occuring peptides
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 7 - protein, 11 aa, unidentified
  source 1..11: note = Naturally occuring peptides
YGRKKRRQRRR

SEQ ID NO: 8 - protein, 12 aa, unidentified
  source 1..12: note = Naturally occuring peptides
RRQRRTSKLMKR

SEQ ID NO: 9 - protein, 27 aa, unidentified
  source 1..27: note = Naturally occuring peptides
GWTLNSAGYLLGKINLKALAALAKKIL

SEQ ID NO: 10 - protein, 33 aa, unidentified
  source 1..33: note = Naturally occuring peptides
KALAWEAKLAKALAKALAKHLAKALAKALKCEA

SEQ ID NO: 11 - protein, 16 aa, unidentified
  source 1..16: note = Naturally occuring peptides
RQIKIWFQNRRMKWKK

SEQ ID NO: 12 - protein, 11 aa, unidentified
  source 1..11: note = Naturally occuring peptides
YGRKKRRQRRR

SEQ ID NO: 13 - protein, 9 aa, unidentified
  source 1..9: note = Naturally occuring peptides
RKKRRQRRR

SEQ ID NO: 14 - protein, 11 aa, unidentified
  source 1..11: note = Naturally occuring peptides
YGRKKRRQRRR

SEQ ID NO: 15 - protein, 8 aa, unidentified
  source 1..8: note = Naturally occuring peptides
RKKRRQRR

SEQ ID NO: 16 - protein, 11 aa, unidentified
  source 1..11: note = Naturally occuring peptides
YARAAARQARA

SEQ ID NO: 17 - protein, 11 aa, unidentified
  source 1..11: note = Naturally occuring peptides
THRLPRRRRRR

SEQ ID NO: 18 - protein, 11 aa, unidentified
  source 1..11: note = Naturally occuring peptides
GGRRARRRRRR